(19) United States
(12) Patent Application Publication
(10) Pub. No.: US 2026/0007452 A1
(43) Pub. Date: Jan. 8, 2026

(54) SURGICAL DEVICE FOR ELECTROCAUTERY EQUIPPED WITH SURGICAL INSTRUMENT

(71) Applicant: LivsMed Inc., Seongnam-si (KR)

(72) Inventor: Ik Jae JANG, Seongnam-si (KR)

(73) Assignee: LivsMed Inc., Seongnam-si (KR)

(21) Appl. No.: 18/916,540

(22) Filed: Oct. 15, 2024

(30) Foreign Application Priority Data
Jul. 4, 2024 (KR) 10-2024-0088136

(51) Int. Cl. A61B 18/14; A61B 18/00

(52) U.S. Cl. CPC A61B 18/1445; A61B 2018/00595; A61B 2018/00601; A61B 2018/00708; A61B 2018/00875; A61B 2018/1452

(57) ABSTRACT

Disclosed is a surgical device for electrocautery. The device includes: at least one energy transmission unit configured to transmit electric energy to a target tissue to cauterize the target tissue; an optical unit configured to irradiate light of a predetermined wavelength band to the target tissue and collect response light from the target tissue; and a control unit configured to determine tissue component information on the target tissue based on a measurement value for the response light and to control electric energy supplied to the target tissue through the energy transmission unit based on the tissue component information.

### CROSS-REFERENCE TO RELATED APPLICATION

This application is based on and claims priority under 35 USC § 119 to Korean Patent Application No. 10-2024-0088136, filed on Jul. 4, 2024, in the Korean Intellectual Property Office, the disclosure of which is incorporated by reference herein in its entirety.

### BACKGROUND

### 1. Field

The present disclosure relates to a surgical device for electrocautery equipped with a surgical instrument, and more particularly, but not limitedly, to a surgical device for electrocautery for controlling an energy output level to an appropriate level, wherein the surgical device for electrocautery is equipped with a surgical instrument mounted on a robotic arm or manually operable for use in laparoscopic surgery or various other surgeries.

### 2. Description of the Related Art

Surgical operations in many cases require cutting and joining of body tissues including organs, muscular tissues, connective tissues and blood vessels. Over the centuries, sharp blades and sutures have been used for cutting and joining. However, bleeding occurs when cutting body tissues, in particular, relatively highly vascularized tissue during surgical operation. Therefore, doctors have been in need of surgical instruments and methods to slow or reduce bleeding during surgical operations.

Recently, it has become possible to use an electric surgical instrument that uses electric energy to perform certain surgical tasks. For example, in surgical instruments such as graspers, scissors, tweezers, blades, needles, and hooks, electric surgical instruments including one or more electrodes formed to receive electric energy have been developed. Electric energy supplied through the electrodes may be used to coagulate, bond, or cut the patient's body tissues. In particular, when electric energy is used, amputation and hemostasis may be performed at the same time.

Electric surgical instruments are typically divided into two types: monopolar and bipolar. In a monopolar electric surgical instrument, electric energy of a specific polarity is supplied to one or more electrodes of the instrument. In addition, electricity of different polarity is electrically connected to the patient. In a bipolar electric surgical instrument, one or more electrodes are electrically connected to a first polarity electric energy source, and one or more electrodes are electrically connected to a second polarity electric energy source opposite to the first polarity.

The above-mentioned background art is technical information that the inventor has possessed for the derivation of the present disclosure or acquired in the process of derivation of the present disclosure, and cannot necessarily be said to be a known technique disclosed to the general public prior to the filing of the present disclosure.

### SUMMARY

An aspect of the present disclosure is directed to providing a surgical device for electrocautery controlling an energy output level to an appropriate level, wherein the surgical device for electrocautery is equipped with a surgical instrument mounted on a robotic arm or manually operable for use in laparoscopic surgery or various other surgeries.

In addition, an aspect of the present disclosure is directed to providing a method for controlling the surgical device for electrocautery and a computer-readable recording medium for recording a program for executing the method on a computer.

The aspects of the present disclosure are not limited to those mentioned above, and other aspects and benefits not mentioned herein will be understood from the following description, and will become apparent from the embodiments of the present disclosure. It is also to be understood that the aspects and benefits of the present disclosure may be realized by means and combinations thereof set forth in claims.

The surgical device for electrocautery according to an embodiment of the present disclosure may include: at least one energy transmission unit configured to transmit electric energy to a target tissue to cauterize the target tissue; an optical unit configured to irradiate light of a predetermined wavelength band to the target tissue and collect response light from the target tissue; and a control unit configured to determine tissue component information on the target tissue based on a measurement value for the response light and to control electric energy supplied to the target tissue through the energy transmission unit based on the tissue component information.

According to an aspect, the target tissue includes a first component, the first component corresponds to a first wavelength, and the control unit may be configured to determine content information on the first component of the target tissue based on an intensity of light of the first wavelength irradiated to the target tissue and an intensity of light of the first wavelength of the response light from the target tissue.

According to an aspect, the target tissue includes a first component and a second component, and the control unit may be configured to increase a supply level of the electric energy in response to an increase in a content of a first component of the target tissue, and to decrease the supply level of the electric energy in response to an increase in a content of a second component of the target tissue.

According to an aspect, the first component may include moisture, and the second component may include fat.

According to an aspect, the moisture may correspond to a wavelength band of 1,010 nm to 1,460 nm, and the fat may correspond to a wavelength band of 900 nm to 950 nm.

According to an aspect, the first wavelength may be configured to decrease with the passage of electric energy irradiation time to the target tissue.

According to an aspect, the control unit may be configured to determine an impedance of the target tissue, and control the electric energy based on the impedance of the target tissue and the tissue component information.

According to an aspect, the control unit may be configured to adjust an electric energy level corresponding to the impedance of the target tissue based on the tissue component information.

According to an aspect, the target tissue may include a first component and a second component, and the control unit may determine a reference electric energy based on the impedance of the target tissue, and determine the electric energy supplied to the energy transmission unit by increasing the reference electric energy based on information on a content of the first component or decreasing the reference electric energy based on information on a content of the second component.

According to an aspect, the target tissue may include a first component, and the control unit may be configured to stop supplying electric energy to the energy transmission unit in response to a determination that the impedance of the target tissue is less than or equal to a predetermined first threshold value and a content of the first component is less than or equal to a predetermined second threshold value.

According to an aspect, there may be further included a first jaw for gripping the target tissue; and a second jaw, wherein the energy transmission unit may include a first electrode provided in the first jaw and a second electrode provided in the second jaw.

According to an aspect, there may be further included a first jaw and a second jaw for gripping the target tissue, wherein the optical unit may be disposed in either the first jaw or the second jaw and configured to irradiate light of the predetermined wavelength band to the target tissue and collect reflected light from the target tissue.

According to an aspect, there may be further included a blade that cut the target tissue while moving between a proximal end and a distal end of either the first jaw or the second jaw.

According to an aspect, the optical unit may be disposed in the first jaw, and the blade may be disposed in the second jaw.

According to an aspect, the optical unit may be disposed in a distal end of either the first jaw or the second jaw.

According to an aspect, the optical unit may include: a light emitting unit that irradiates light of the predetermined wavelength band to the target tissue; and at least one absorption unit that is disposed around the light emitting unit and configured to collect reflected light from the target tissue.

According to an aspect, there may be further included a spectrometer that generates dispersed light of the predetermined wavelength band and performs a measurement on the response light, wherein the optical unit may be connected to the spectrometer based on an optical fiber.

According to an aspect, there may be further included a power supply unit that supplies electric energy to the energy transmission unit, wherein the energy transmission unit may be connected to the power supply unit based on a power line.

According to an aspect, the optical fiber and the power line may be configured as a single cable having a bundle structure.

A method for determining supply electric energy of a surgical device for electrocautery according to another embodiment of the present disclosure is performed by a computing device, wherein the method may include: irradiating light of a predetermined wavelength band to a target tissue using an optical unit and collecting response light from the target tissue; determining tissue component information on the target tissue based on a measurement value for the response light; determining electric energy supplied to the target tissue through an energy transmission unit based on the tissue component information; and transmitting the determined electric energy to the target tissue using the energy transmission unit.

In addition, another method for implementing an embodiment of the present disclosure, another system, and a computer-readable recording medium storing a computer program for executing the method may be further provided.

Other aspects, features and benefits other than those described above will become apparent from the following drawings, claims and detailed description of the present disclosure.

According to an aspect of the present disclosure, there is provided a surgical device for electrocautery controlling an energy output level to an appropriate level, wherein the surgical device for electrocautery is equipped with a surgical instrument mounted on a robotic arm or manually operable for use in laparoscopic surgery or various other surgeries, thereby minimizing tissue damage and performing rapid sealing of blood vessels.

The benefits of the present disclosure are not limited to those mentioned above, and other benefits not mentioned may be clearly understood by those skilled in the art from the following description.

### BRIEF DESCRIPTION OF THE DRAWINGS

FIG. 1A is a conceptual diagram of a pitch motion of a conventional surgical instrument, and FIG. 1B is a conceptual diagram of a yaw motion.

FIG. 1C is a conceptual diagram of a pitch motion of another conventional surgical instrument, and FIG. 1D is a conceptual diagram of a yaw motion.

FIG. 1E is a conceptual diagram of a pitch motion of surgical instrument according to an embodiment of the present disclosure, and FIG. 1F is a conceptual diagram of a yaw motion.

FIG. 2 is a perspective view illustrating a surgical instrument for electrocautery according to an embodiment of the present disclosure.

FIGS. 3 to 8 are views illustrating an end tool of the surgical instrument for electrocautery of FIG. 2.

FIG. 9 is a perspective view illustrating an end tool hub of the surgical instrument for electrocautery of FIG. 2.

FIGS. 10 and 11 are cut-away perspective views of the end tool hub of FIG. 9.

FIGS. 12 and 13 are perspective views illustrating the end tool hub of FIG. 9.

FIG. 14 is a side view illustrating the end tool hub and the guide tube of FIG. 9.

FIG. 15 is a plan view illustrating the end tool hub and the guide tube of FIG. 9.

FIG. 16A is a perspective view illustrating an actuation hub of the surgical instrument for electrocautery of FIG. 2 and FIG. 9.

FIG. 16B is a cut-away perspective view illustrating an actuation hub of the surgical instrument for electrocautery of FIG. 2 and FIG. 9.

FIG. 17 is a view illustrating a state in which the guide tube, the blade wire, and the blade are mounted on the actuation hub illustrated in the cut-away perspective view of FIG. 16B.

FIG. 18 is an exploded perspective view illustrating the end tool of the surgical instrument for electrocautery of FIG. 2.

FIG. 19 is a perspective view illustrating a first jaw of the end tool of the surgical instrument for electrocautery of FIG. 2.

FIG. 20 is a perspective view illustrating a second jaw of the end tool of the surgical instrument for electrocautery of FIG. 2.

FIG. 21 is a perspective view illustrating a first jaw pulley of the surgical instrument for electrocautery of FIG. 2.

FIG. 22A is a plan view illustrating an opening motion of the first jaw of the end tool of the surgical instrument for electrocautery of FIG. 2.

FIG. 22B is a plan view illustrating a closing motion of the first jaw of the end tool of the surgical instrument for electrocautery of FIG. 2.

FIG. 23A is a plan view illustrating an opening motion of the second jaw of the end tool of the surgical instrument for electrocautery of FIG. 2.

FIG. 23B is a plan view illustrating a closing motion of the second jaw of the end tool of the surgical instrument for electrocautery of FIG. 2.

FIG. 24A is a plan view illustrating an opening motion of the first jaw and the second jaw of the end tool of the surgical instrument for electrocautery of FIG. 2.

FIG. 24B is a plan view illustrating a closing motion of the first jaw and the second jaw of the end tool of the surgical instrument for electrocautery of FIG. 2.

FIGS. 25 and 26 are plan views illustrating an opening and closing motion of the end tool of the surgical instrument for electrocautery of FIG. 2.

FIGS. 27 to 29 are partial cross-sectional views illustrating a motion of a blade of the end tool of the surgical instrument for electrocautery of FIG. 2.

FIGS. 30 and 31 are views illustrating a process of performing an opening and closing motion in a state in which the end tool of the surgical instrument for electrocautery of FIG. 2 is yaw-rotated by −90°.

FIGS. 32 and 33 are bottom views illustrating a process of performing an opening and closing motion in a state in which the end tool of the surgical instrument for electrocautery of FIG. 2 is yaw-rotated by +90°.

FIGS. 34 and 35 are views illustrating a path of the guide tube and a movement path of the blade during a cutting motion in a state in which the end tool of the surgical instrument for electrocautery of FIG. 2 is yaw-rotated.

FIGS. 36 and 37 are views illustrating a process of performing an opening and closing motion in a state in which the end tool of the surgical instrument for electrocautery of FIG. 2 is pitch-rotated by +90°.

FIGS. 38 and 39 are views illustrating a process of performing an opening and closing motion in a state in which the end tool of the surgical instrument for electrocautery of FIG. 2 is pitch-rotated by −90°.

FIG. 40 is a view illustrating a path of the guide tube in a state in which the end tool of the surgical instrument for electrocautery of FIG. 2 is pitch-rotated by −90°.

FIGS. 41 and 42 are views illustrating a path of the guide tube and a movement path of the blade during a cutting motion in a state in which the end tool of the surgical instrument for electrocautery of FIG. 2 is pitch-rotated by −90°.

FIG. 43 is a perspective view illustrating a pitch-rotated state and a yaw-rotated state of the surgical instrument for electrocautery of FIG. 2.

FIGS. 44 to 46 are views illustrating the end tool of the surgical instrument for electrocautery of FIG. 2 performing a cutting motion while simultaneously pitch-rotating by −90° and yaw-rotating by +90°.

FIG. 47 is a perspective view illustrating the surgical instrument for electrocautery of FIG. 2.

FIGS. 48 and 49 are perspective views illustrating a manipulation unit of the surgical instrument for electrocautery of FIG. 2.

FIG. 50 is a view schematically illustrating only the configuration of pulleys and wires configuring joints of the surgical instrument for electrocautery of FIG. 2.

FIG. 51 is a block diagram illustrating an exemplary configuration of a surgical device for electrocautery according to an embodiment of the present disclosure.

FIG. 52 is an exemplary implementation example of the surgical device for electrocautery of FIG. 51.

FIG. 53 is a conceptual diagram of a distal disposition of an optical unit.

FIG. 54 is an exemplary diagram of the disposition of a light-emitting unit and a light-absorption unit of the optical unit.

FIG. 55 is a schematic flow diagram of a method for determining supply electric energy of the surgical device for electrocautery according to an embodiment of the present disclosure.

FIG. 56 shows an optical absorption coefficient according to a tissue component of an exemplary target tissue.

FIG. 57 shows the relationship between the tissue change due to heat and the optical reflectivity and impedance accordingly.

### DETAILED DESCRIPTION

Hereinafter, various embodiments of the present disclosure will be described with reference to the accompanying drawings. As the present disclosure allows for various changes and numerous embodiments, certain embodiments will be illustrated in the drawings and described in the detailed description. However, various embodiments are not intended to limit the present disclosure to certain embodiments, and should be construed as including all changes, equivalents, and/or alternatives included in the spirit and scope of various embodiments of the present disclosure. With regard to the description of the drawings, similar reference numerals may be used to refer to similar components.

Expressions such as “include” or “may include” that may be used in various embodiments of the present disclosure specify the presence of a corresponding function, operation, or component, and do not preclude the presence or addition of one or more functions, operations, or components. In addition, it will be understood that terms such as “include” or “comprise” as used in various embodiments of the present disclosure specify the presence of stated features, numbers, steps, operations, components, parts, and combinations thereof, but do not preclude in advance the presence or addition of one or more other features, numbers, steps, operations, components, parts, combinations thereof.

A term such as “or” as used in various embodiments of the present disclosure may include any and all possible combinations of words listed together. For example, an expression such as “A or B” may include “A,” “B,” or both “A” and “B.”

Expressions such as “first,” “second,” “primarily,” or “secondarily” as used in various embodiments of the present disclosure may represent various components and do not limit corresponding components. For example, the aforementioned expressions do not limit the order and/or importance of the corresponding components. The aforementioned expressions may be used to distinguish one component from another. For example, both a first user device and a second user device refer to user devices and represent different user devices. For example, without departing from the scope of various embodiments of the present disclosure, a first component may be referred to as a second component, and similarly, a second component may be referred to as a first component.

Terms such as “module,” “unit,” and “part” as used in the embodiments of the present disclosure refer to components that perform at least one function or operation, and the components may be implemented as hardware or software or as a combination of hardware and software. In addition, a plurality of “modules,” “units,” and “parts” may be integrated into at least one module or chip and implemented as at least one processor, except when each of the modules, units, and parts needs to be implemented as individual specific hardware.

Terms used in various embodiments of the present disclosure are merely used to describe certain embodiments, and are not intended to limit various embodiments of the present disclosure. As used herein, the singular forms are intended to include the plural forms as well, unless the context clearly indicates otherwise.

Unless otherwise defined, all terms used herein including technical or scientific terms have the same meanings as commonly understood by those of ordinary skill in the art to which various embodiments of the present disclosure pertain.

Terms such as those defined in commonly used dictionaries should be interpreted as having meanings consistent with the meanings in the context of the related art, and should not be interpreted in an idealized or overly formal sense, unless explicitly defined in various embodiments of the present disclosure.

Hereinafter, various embodiments of the present disclosure will be described in detail with reference to the accompanying drawings.

In a surgical instrument for electrocautery according to an aspect of the present disclosure, when a manipulation unit is rotated in one direction for at least any one of pitch, yaw, and actuation motions, an end tool is rotated in intuitively the same direction as a direction in which the manipulation unit is manipulated.

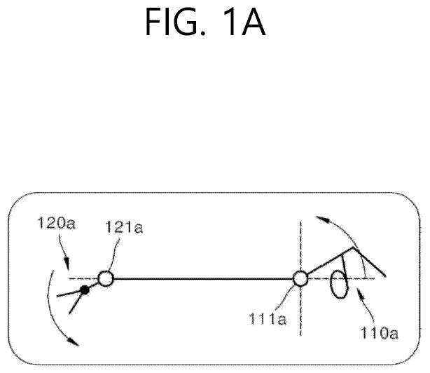

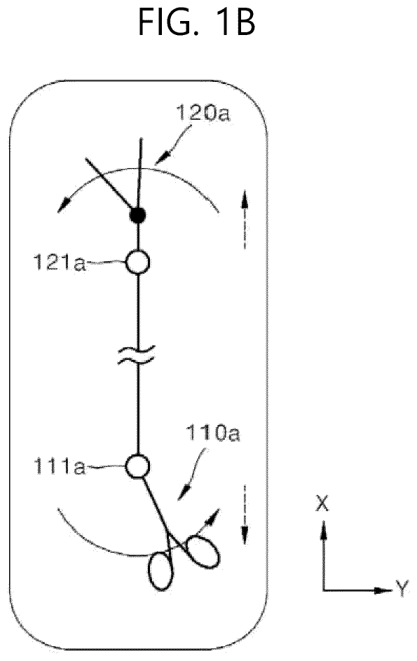

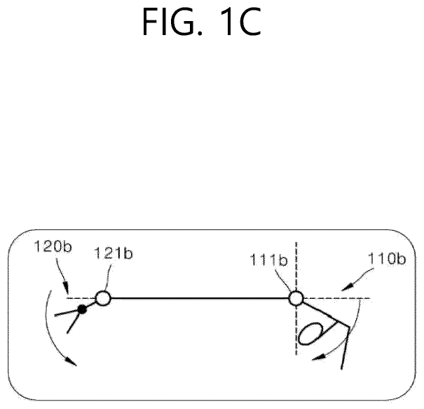

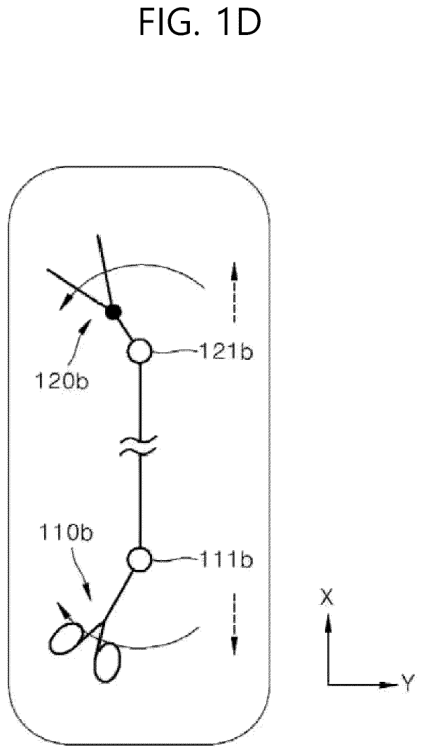

FIG. 1A is a conceptual diagram of a pitch motion of a conventional surgical instrument, and FIG. 1B is a conceptual diagram of a yaw motion thereof.

Referring to FIG. 1A, in performing a pitch motion of a conventional surgical instrument, in a state in which an end tool 120a is formed in front of a rotation center 121a of the end tool, and a manipulation unit 110a is formed at the rear of a rotation center 111a of the manipulation unit, when the manipulation unit 110a is rotated in a clockwise direction, the end tool 120a is also rotated in the clockwise direction, and when the manipulation unit 120a is rotated in a counterclockwise direction, the end tool 120a is also rotated in the counterclockwise direction. Referring to FIG. 1B, in performing a yaw motion of the conventional surgical instrument, in a state in which the end tool 120a is formed in front of the rotation center 121a of the end tool, and the manipulation unit 110a is formed at the rear of the rotation center 111a of the manipulation unit, when the manipulation unit 110a is rotated in the clockwise direction, the end tool 120a is also rotated in the clockwise direction, and when the manipulation unit 120a is rotated in the counterclockwise direction, the end tool 120a is also rotated in the counterclockwise direction. In this connection, in view of left and right directions of a user, when the user moves the manipulation unit 110a to the left, the end tool 120a is moved to the right, and when the user moves the manipulation unit 110a to the right, the end tool 120a is moved to the left. As a result, a manipulation direction of the user and a motion direction of the end tool are opposite to each other, which may cause the user to make a mistake, and user's manipulation may not be easy.

FIG. 1C is a conceptual diagram of a pitch motion of another conventional surgical instrument, and FIG. 1D is a conceptual diagram of a yaw motion thereof.

Referring to FIG. 1C, in the conventional surgical instrument, which is partially formed in a mirror symmetrical shape, in performing a pitch motion, in a state in which an end tool 120b is formed in front of a rotation center 121b of the end tool, and a manipulation unit 110b is formed at the rear of a rotation center 111b of the manipulation unit, when the manipulation unit 110b is rotated in the clockwise direction, the end tool 120b is rotated in the counterclockwise direction, and when the manipulation unit 110b is rotated in the counterclockwise direction, the end tool 120b is rotated in the clockwise direction. In this connection, in view of rotation directions of the manipulation unit and the end tool, a rotation direction in which the user rotates the manipulation unit 110b and a rotation direction of the end tool 120b according thereto are opposite to each other. As a result, the user may be confused with the manipulation direction, and as the motion of a joint is not intuitive, the user may make an error. In addition, referring to FIG. 1D, in performing a yaw motion, in a state in which the end tool 120b is formed in front of the rotation center 121b of the end tool, and the manipulation unit 110b is formed at the rear of the rotation center 111b of the manipulation unit, when the manipulation unit 110b is rotated in the clockwise direction, the end tool 120b is rotated in the counterclockwise direction, and when the manipulation unit 110b is rotated in the counterclockwise direction, the end tool 120b is rotated in the clockwise direction. In this connection, in view of rotation directions of the manipulation unit and the end tool, a rotation direction in which the user rotates the manipulation unit 110b and a rotation direction of the end tool 120b according thereto are opposite to each other. As a result, the user may be confused with the manipulation direction, and as the motion of the joint is not intuitive, the user may make an error. In the user's pitch or yaw manipulation of the conventional surgical instrument, the user's manipulation direction and the end tool's motion direction do not match each other in view of one of the rotation direction and the left and right directions. This is because the configurations of the end tool and the manipulation unit are different from each other in the joint configuration of the conventional surgical instrument. In other words, this is because the manipulation unit is formed at the rear of the rotation center of the manipulation unit, while the end tool is formed in front of the rotation center of the end tool. In order to address the above issues, in a surgical instrument according to an embodiment of the present disclosure, which is illustrated in FIGS. 1E and 1F, an end tool 120c is formed in front of a rotation center 121c of the end tool and a manipulation unit 110c is also formed in front of a rotation center 111c of the manipulation unit, so that the motions of the manipulation unit 110c and the end tool 120c are intuitively matched with each other. In other words, unlike existing examples such as those shown in FIGS. 1A, 1B, 1C, and 1D, in which the manipulation unit is close to a user with respect to the joint thereof (in other words, away from the end tool), the surgical instrument according to an embodiment of the present disclosure, which is illustrated in FIGS. 1E and 1F, is formed such that at least a portion of the manipulation unit is closer (than a joint thereof) to the end tool with respect to the joint thereof at any one moment or more in a manipulation process.

In other words, in the conventional surgical instrument as illustrated in FIGS. 1A, 1B, 1C, and 1D, the manipulation unit is formed at the rear of the rotation center thereof, while the end tool is located in front of the rotation center thereof, and thus the end tool is moved at a front side thereof with a rear side fixed through a motion of the manipulation unit that is moved at a rear side thereof with a front side thereof fixed, which is not an intuitively matching structure. Accordingly, a mismatch may occur between the manipulation of the manipulation unit and the motion of the end tool in view of the left and right directions or in view of the rotation direction, which may cause confusion to the user, and the manipulation of the manipulation unit may be difficult to perform intuitively and quickly and may cause mistakes. In contrast, in the surgical instrument according to an embodiment of the present disclosure, since both the end tool and the manipulation unit are moved with respect to the rotation center formed at the rear side thereof, the motions are intuitively matched with each other in terms of structure. In other words, moving portions of the manipulation unit are moved with respect to the rotation center formed at the rear side thereof just as moving portions of the end tool are moved with respect to the rotation center formed at the rear side thereof, and thus the motions are intuitively matched with each other in terms of structure. This allows the user to intuitively and quickly perform a control in a direction toward the end tool, and a possibility of making a mistake may be significantly reduced.

### Surgical Instrument for Electrocautery

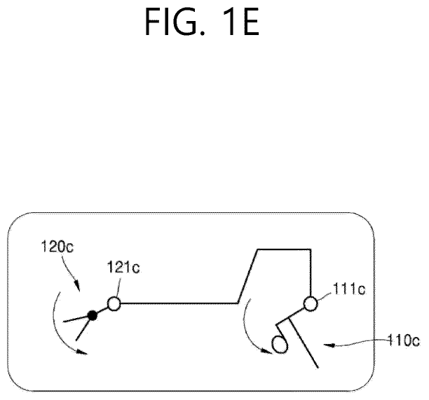

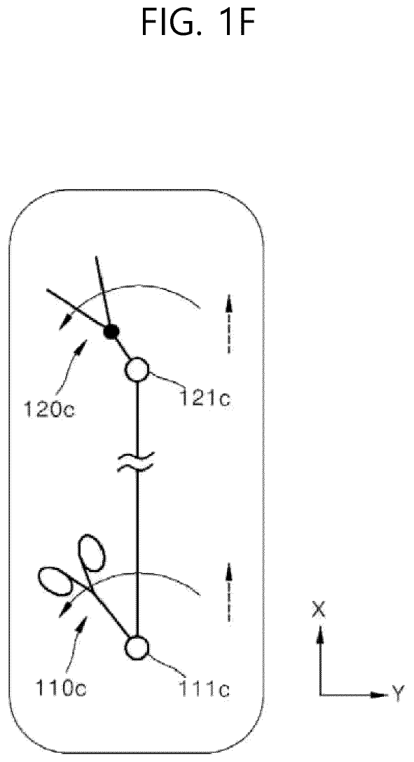

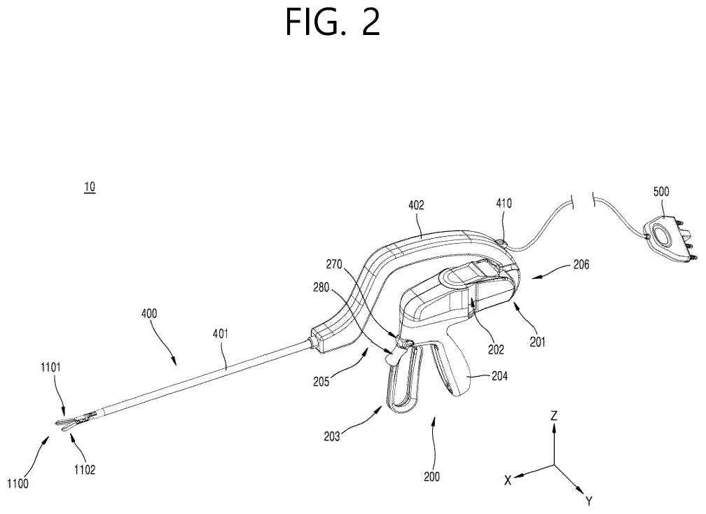

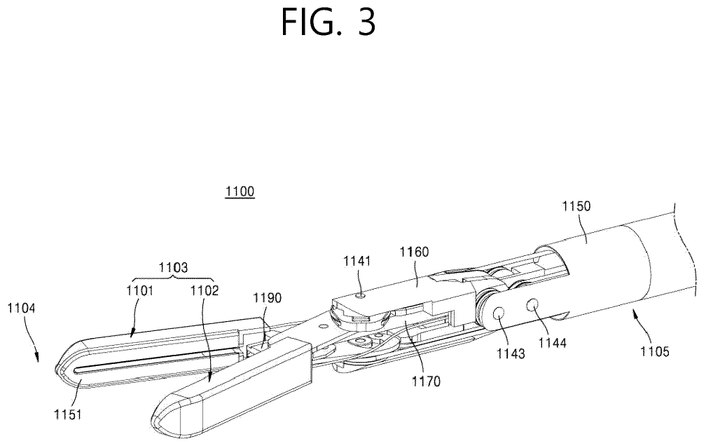

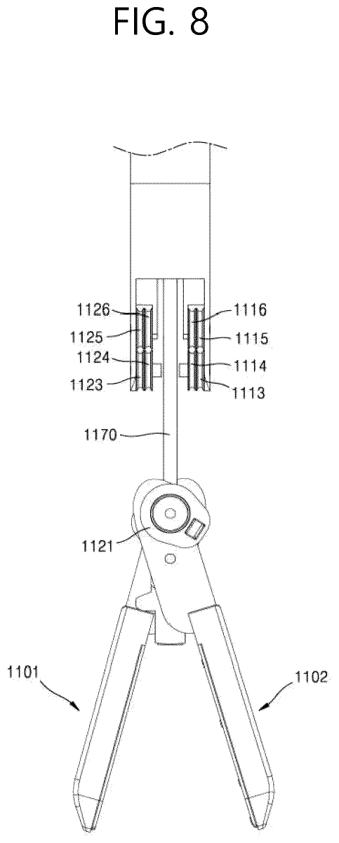

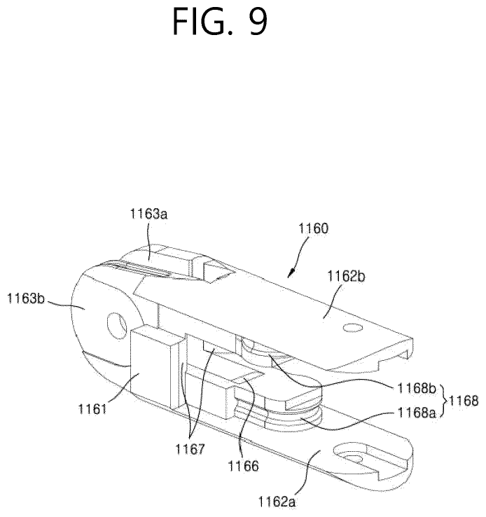

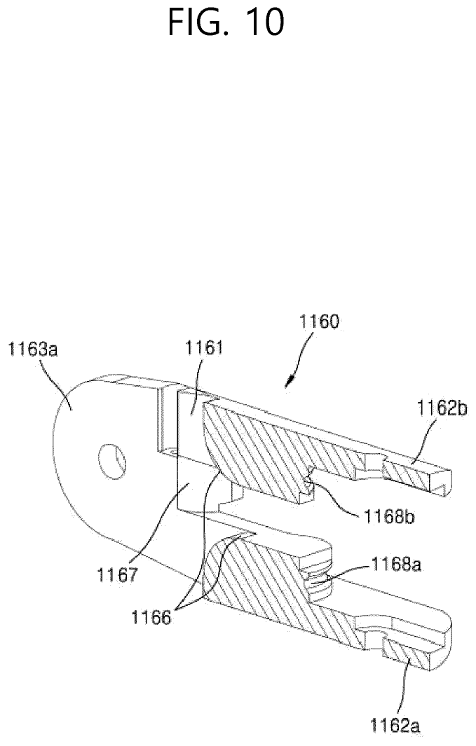

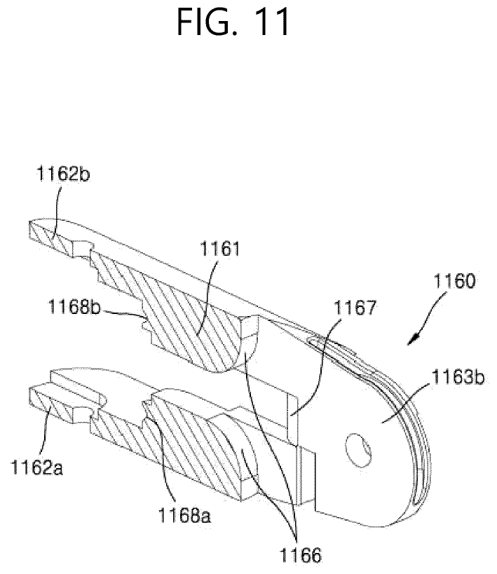

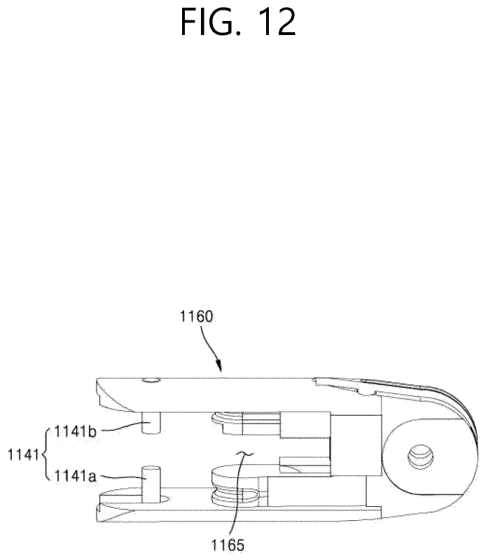

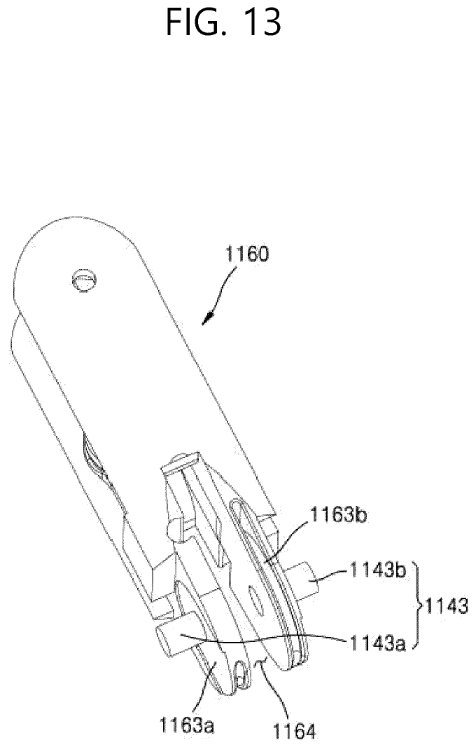

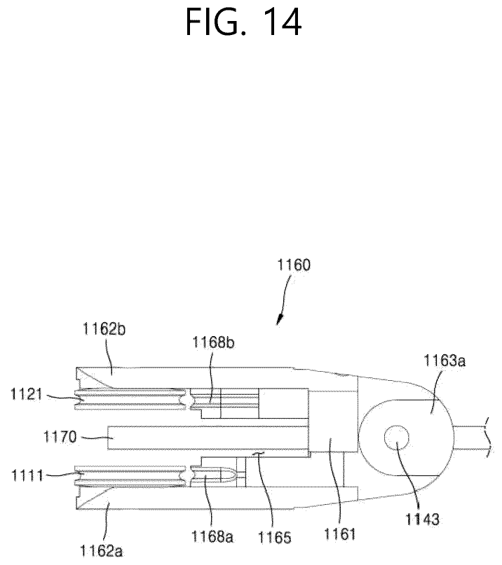

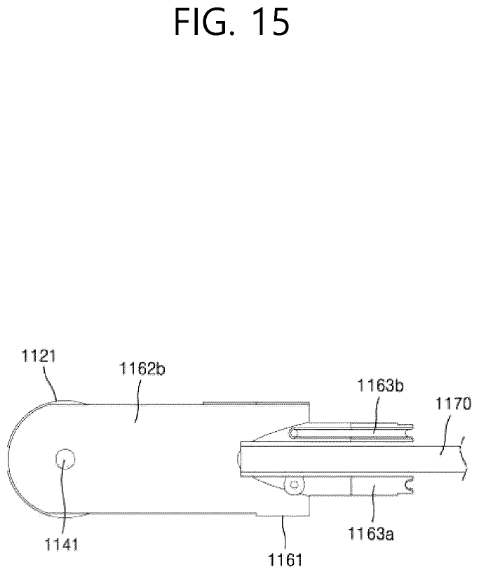

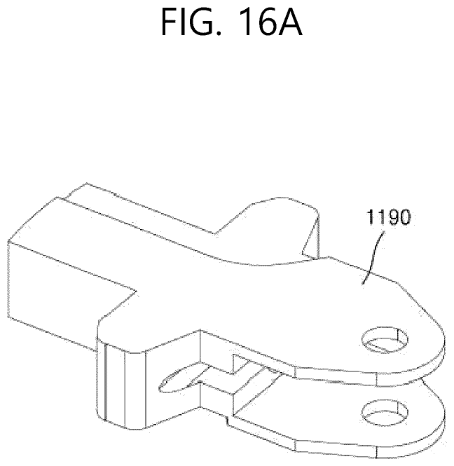

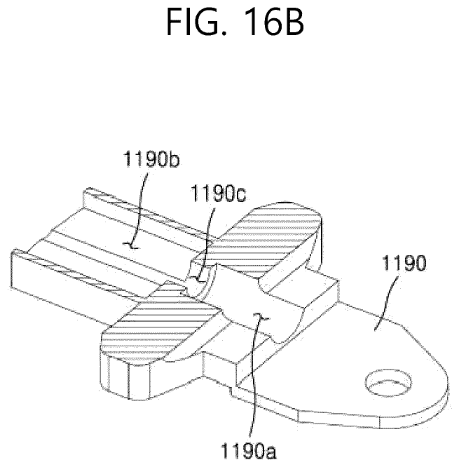

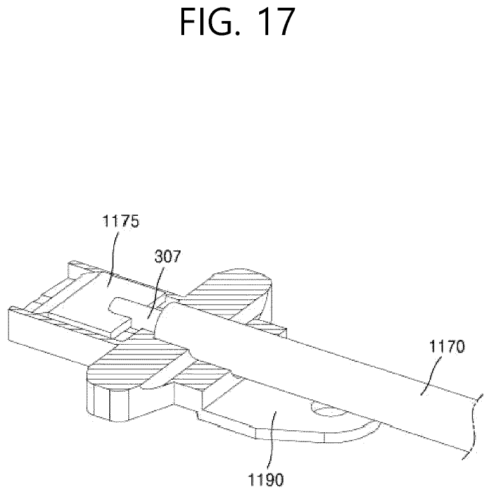

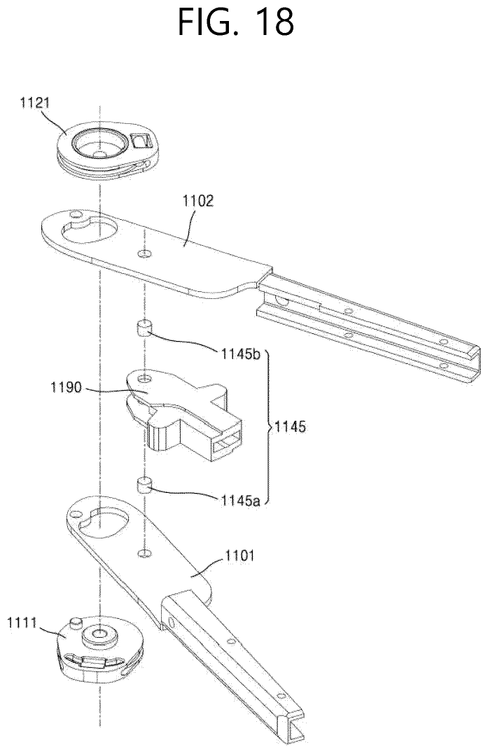

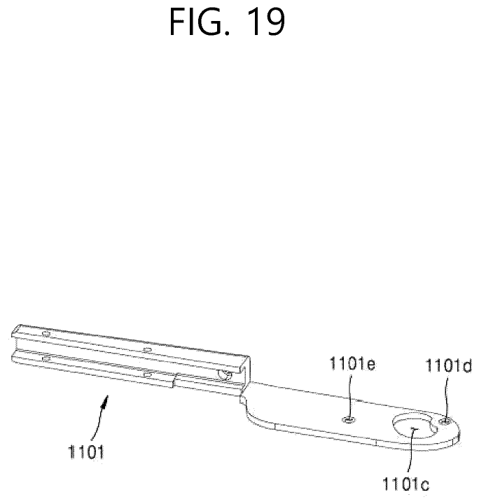

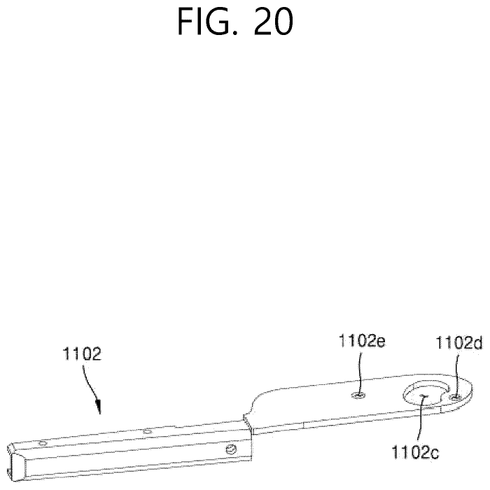

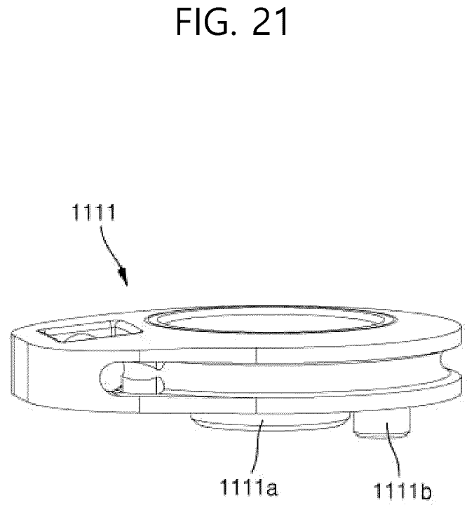

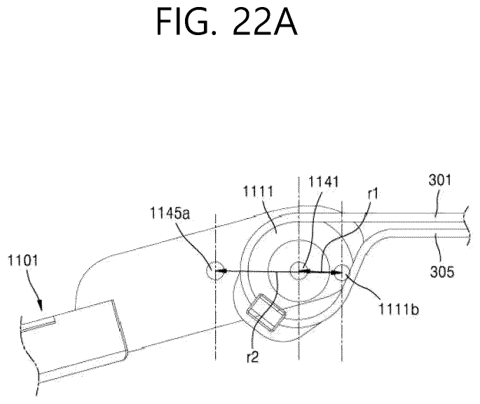

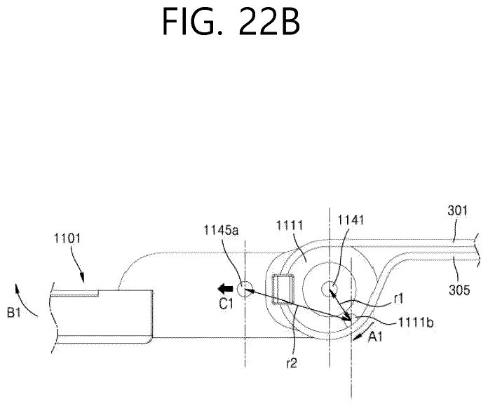

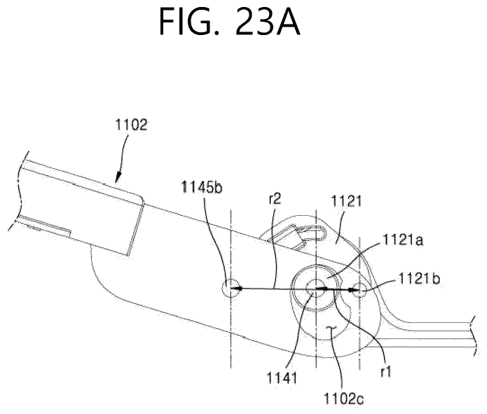

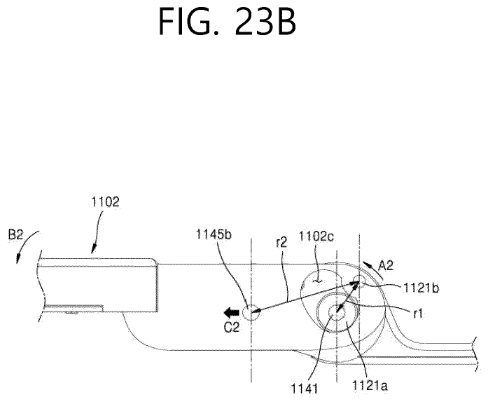

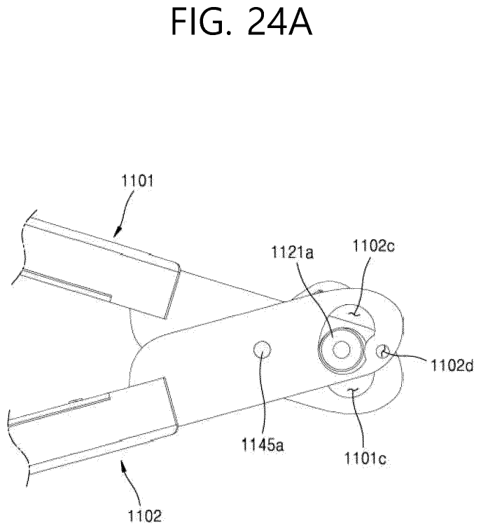

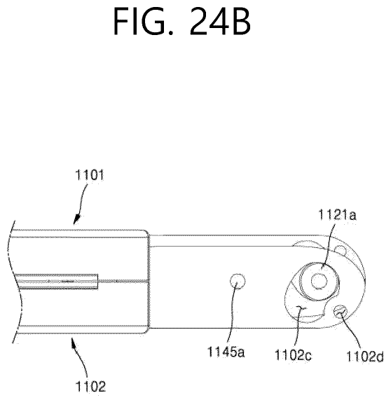

FIG. 2 is a perspective view illustrating a surgical instrument for electrocautery according to an embodiment of the present disclosure. FIGS. 3 to 8 are views illustrating an end tool of the surgical instrument for electrocautery of FIG. 2. FIG. 9 is a perspective view illustrating an end tool hub of the surgical instrument for electrocautery of FIG. 2. FIGS. 10 and 11 are cut-away perspective views of the end tool hub of FIG. 9. FIGS. 12 and 13 are perspective views illustrating the end tool hub of FIG. 9. FIG. 14 is a side view illustrating the end tool hub and the guide tube of FIG. 9. FIG. 15 is a plan view illustrating the end tool hub and the guide tube of FIG. 9. FIG. 16A is a perspective view illustrating an actuation hub of the surgical instrument for electrocautery of FIG. 2 and FIG. 9. FIG. 16B is a cut-away perspective view illustrating an actuation hub of the surgical instrument for electrocautery of FIG. 2 and FIG. 9. FIG. 17 is a view illustrating a state in which the guide tube, the blade wire, and the blade are mounted on the actuation hub illustrated in the cut-away perspective view of FIG. 16B. FIG. 18 is an exploded perspective view illustrating the end tool of the surgical instrument for electrocautery of FIG. 2. FIG. 19 is a perspective view illustrating a first jaw of the end tool of the surgical instrument for electrocautery of FIG. 2. FIG. 20 is a perspective view illustrating a second jaw of the end tool of the surgical instrument for electrocautery of FIG. 2. FIG. 21 is a perspective view illustrating a first jaw pulley of the surgical instrument for electrocautery of FIG. 2. FIG. 22A is a plan view illustrating an opening of the first jaw of the end tool of the surgical instrument for electrocautery of FIG. 2. FIG. 22B is a plan view illustrating a closing motion of the first jaw of the end tool of the surgical instrument for electrocautery of FIG. 2. FIG. 23A is a plan view illustrating an opening motion of the second jaw of the end tool of the surgical instrument for electrocautery of FIG. 2. FIG. 23B is a plan view illustrating a closing motion of the second jaw of the end tool of the surgical instrument for electrocautery of FIG. 2. FIG. 24A is a plan view illustrating an opening motion of the first jaw and the second jaw of the end tool of the surgical instrument for electrocautery of FIG. 2. FIG. 24B is a plan view illustrating a closing motion of the first jaw and the second jaw of the end tool of the surgical instrument for electrocautery of FIG. 2.

Referring to FIGS. 2 to 24, a surgical instrument 10 for electrocautery according to an embodiment of the present disclosure includes an end tool 1100, a manipulation unit 200, a power transmission unit 300, and a connection unit 400.

Herein, the connection unit 400 is formed in the shape of a hollow shaft, and one or more wires and electric wires may be accommodated therein. The manipulation unit 200 is coupled to one end portion of the connection unit 400, the end tool 1100 is coupled to the other end portion thereof, and the connection unit 400 may serve to connect the manipulation unit 200 and the end tool 1100. Herein, the connection unit 400 of the surgical instrument 10 for electrocautery according to an embodiment of the present disclosure includes a straight unit 401 and a bent unit 402, wherein the straight unit 401 is formed at a side coupled to the end tool 1100, and the bent portion 402 is formed at a side to which the manipulation unit 200 is coupled. As such, since the end portion of the connection unit 400 at the side of the manipulation unit 200 is formed to be bent, a pitch manipulation unit 201, a yaw manipulation unit 202, and an actuation manipulation unit 203 may be formed along an extension line of the end tool 1100 or adjacent to the extension line. In other words, the pitch manipulation unit 201 and the yaw manipulation unit 202 are at least partially accommodated in a concave portion formed by the bent unit 402. Due to the shape of the bent portion 402, the shapes and motions of the manipulation unit 200 and the end tool 1100 may be further intuitively matched with each other.

A plane on which the bent unit 402 is formed may be substantially the same as a pitch plane, that is, an XZ plane of FIG. 2. As such, as the bent unit 402 is formed on substantially the same plane as the XZ plane, interference with the manipulation unit may be reduced. For intuitive motions of the end tool and the manipulation unit, any form other than the XZ plane may be possible.

A connector 410 may be formed on the bent unit 402. The connector 410 may be connected to an external power supply (not shown), and the connector 410 may be connected to a jaw 1103 through electric wires 411 and 412 to transfer electric energy supplied from the external power supply (not shown) to the jaw 1103. Here, the connector 410 may be of a bipolar-type having two electrodes, or the connector 410 may be of a monopolar type having one electrode. According to an aspect, the connector 410 may be provided with a contact unit 500 to provide convenience of connection to the external power supply (not shown).

The manipulation unit 200 is formed at the one end portion of the connection unit 400 and provided as an interface to be directly controlled by a medical doctor, for example, a tongs shape, a stick shape, a lever shape, or the like, and when the medical doctor controls the manipulation unit 200, the end tool 1100, which is connected to the interface and inserted into the body of a surgical patient, performs a certain operation, thereby performing surgery. Herein, the manipulation unit 200 is illustrated in FIG. 2 as being formed in a handle shape that is rotatable while the finger is inserted therein, the concept of the present disclosure is not limited thereto, and various types of manipulation units that are connected to the end tool 1100 and manipulate the end tool 1100 may be possible.

The end tool 1100 is formed on the other end portion of the connection unit 400, and performs necessary motions for surgery by being inserted into a surgical site. In an example of the end tool 1100 described above, as shown in FIGS. 2 to 3, a pair of jaws 1103 for performing a grip motion may be used. However, the concept of the present disclosure is not limited thereto, and various devices for performing surgery may be used as the end tool 1100. For example, a configuration of a cantilever cautery may also be used as the end tool. The end tool 1100 is connected to the manipulation unit 200 by the power transmission unit 300, and receives a driving force of the manipulation unit 200 through the power transmission unit 300 to perform a motion necessary for surgery, such as gripping, cutting, suturing, or the like.

Herein, the end tool 1100 of the surgical instrument 10 for electrocautery according to the fourth embodiment of the present disclosure is formed to be rotatable in at least one direction, for example, the end tool 1100 may perform a pitch motion around a Y-axis of FIG. 2 and simultaneously perform a yaw motion and an actuation motion around a Z-axis of FIG. 2.

The power transmission unit 300 may connect the manipulation unit 200 to the end tool 1100, transmit the driving force of the manipulation unit 200 to the end tool 1100, and include a plurality of wires, pulleys, links, sections, gears, or the like.

The end tool 1100, the manipulation unit 200, the power transmission unit 300, and the like of the surgical instrument 10 for electrocautery of FIG. 2 will be described in detail later.

### Power Transmission Unit

Hereinafter, the power transmission unit 300 of the surgical instrument 10 for electrocautery of FIG. 2 will be described in more detail.

With reference to FIGS. 2 to 8, the power transmission unit 300 of the surgical instrument 10 for electrocautery according to an embodiment of the present disclosure may include a wire 301, a wire 302, a wire 303, a wire 304, a wire 305, a wire 306, and a blade wire 307.

Herein, the wire 301 and the wire 305 may form a pair and serve as a first jaw wire. The wire 302 and the wire 306 may form a pair to serve as a second jaw wire. Herein, a component encompassing the wire 301 and the wire 305, which are the first jaw wire, and the wire 302 and the wire 306, which are the second jaw wire may be referred to as a jaw wire. In addition, the wire 303 and the wire 304 may form a pair to serve as a pitch wire.

In addition, the power transmission unit 300 of the surgical instrument 10 for electrocautery according to an embodiment of the present disclosure may include a fastening member 321, a fastening member 323, a fastening member 324, a fastening member 326, and a fastening member 327, which are coupled to each end portion of the respective wires to combine the wires with the pulleys. Herein, each fastening member may have various shapes as needed, such as a ball shape, a tube shape, etc.

Herein, on the end tool 1100's side, the fastening member 321/the fastening member 322 may serve as a pitch wire-end tool fastening member, the fastening member 323 may serve as a first jaw wire-end tool fastening member, and the fastening member 326 may serve as a second jaw wire-end tool fastening member.

In addition, on the manipulation unit 200's side, the fastening member 324 may serve as a first jaw wire-manipulation unit fastening member, and the fastening member 327 may serve as a second jaw wire-manipulation unit fastening member. Furthermore, although it is not shown in the drawings, a pitch wire-manipulation unit fastening member and a blade wire-manipulation unit fastening member may be further formed on the manipulation unit 200's side.

The combination relation among the wires, the fastening members, and each pulley is described in detail below.

First, the wire 301 and the wire 305, which are the first jaw wire, may be a single wire. The fastening member 323, which is the first jaw wire-end tool fastening member, may be fit into a middle point of the first jaw wire, a single wire. When the fastening member 323 is fixed through crimping, two strands of the first jaw wire based on the fastening member 323 may be referred to as the wire 301 and the wire 305, respectively.

Alternatively, the wire 301 and the wire 305, which are the first jaw wire, may be formed as separate wires, and the wire 301 and the wire 305 may be connected to each other by the fastening member 323.

In addition, as the fastening member 323 is coupled to a pulley 1111, the wire 301 and the wire 305 may be fixedly coupled to the pulley 1111. In this manner, the pulley 1111 may rotate as the wire 301 and the wire 305 are pulled and unwound.

In the wire 301 and the wire 305, the first jaw wire-manipulation portion fastening member 324 may be coupled to an end portion opposite to the end portion to which the fastening member 323 is coupled.

In addition, as the first jaw wire-manipulation portion fastening member 324 is coupled to a pulley 210, the wire 301 and the wire 305 may be fixedly coupled to the pulley 211. As a result, when the pulley 211 is rotated by a motor or human force, the pulley 1111 of the end tool 1100 may rotate as the wire 301 and the wire 305 are pulled and unwound.

Similar to the above, each of the wire 302 and the wire 306, which are the second jaw wire, may be coupled to the second jaw wire-end tool fastening member 326 and the second jaw wire-manipulation portion fastening member 327, respectively. In addition, the fastening member 326 may be coupled to a pulley 1121, and the second jaw wire-manipulation portion fastening member 327 may be coupled to a pulley 220. As a result, when the pulley 220 is rotated by a motor or human force, the pulley 1121 of the end tool 1100 may rotate as the wire 302 and the wire 306 are pulled and unwound.

Similar to the above, the wire 304, which is the pitch wire, is coupled to the fastening member 321, which is a pitch wire-end tool fastening member, and the pitch wire-manipulation unit fastening member (not shown). In addition, the wire 303, which is the pitch wire, is coupled to the fastening member 322, which is a pitch wire-end tool fastening member, and the pitch wire-manipulation unit fastening member (not shown).

In addition, the fastening member 321 is coupled to a first pitch pulley unit 1163a of an end tool hub 1160, the fastening member 322 is coupled to a second pitch pulley unit 1163b of the end tool hub 1160, and the pitch wire-manipulation unit fastening member (not shown) is coupled with a pulley 231. As a result, when the pulley 231 is rotated by a motor or human force, the end tool hub 1160 of the end tool 1100 may rotate as the wire 303 and the wire 304 are pulled and unwound.

In explaining an embodiment of the present disclosure, a part close to a user, that is, a part close to the manipulation unit 200, is described as a proximal end, and a part far from the user, that is, a part close to the end tool 1100, is described as a distal end.

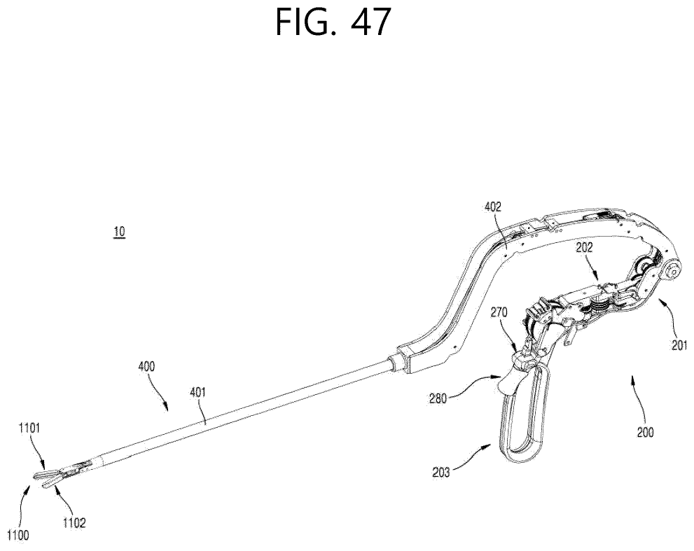

For example, referring to FIGS. 3 and 47, in the end tool 1100, the part closer to the manipulation unit 200 is defined as a proximal end 1105 of the end tool 1100, and the part farther from the manipulation unit 200, that is, the part closer to an end portion of the end tool 1100, is defined as a distal end 1104 of the end tool 1100. To explain this from another perspective, the proximal end 1105 of the end tool 1100 may be described as the part closer to the connection unit 400, and the distal end 1104 of the end tool 1100 may be described as the part farther from the connection unit 400.

One end portion of the blade wire 307 is coupled to a blade 1175 to be described later, and the other end portion thereof is coupled to a cutting manipulation unit 280 of the manipulation unit 200. By the manipulation of the cutting manipulation unit 280, a cutting motion may be performed as the blade wire 307 is moved from the proximal end 1105 toward the distal end 1104 of the end tool 1100, or the blade wire 307 may return from the distal end 1104 toward the proximal end 1105 of the end tool 1100.

In this connection, at least a portion of the blade wire 307 may be accommodated in a guide tube 1170 to be described later. Accordingly, when the guide tube 1170 is bent in response to a pitch motion or yaw motion of the end tool 1100, the blade wire 307 accommodated therein may also be bent together with the guide tube 1170. The guide tube 1170 will be described in more detail later.

In addition, the blade wire 307 is formed in a longitudinal direction of the connection unit 400 to be linearly movable in the connection unit 400. In addition, since one end portion of the blade wire 307 is coupled to the blade 1175, when the blade wire 307 is linearly moved in the longitudinal direction of the connection unit 400, the blade 1175 connected thereto is also linearly moved. In other words, when the blade wire 307 is linearly moved in the longitudinal direction of the connection unit 400, a cutting motion is performed as the blade 1175 connected thereto is moved toward the distal end 1104 or the proximal end 1105 of the end tool 1100. This will be described in more detail later.

The member that moves linearly by the blade wire 307 within the end tool 1100 is not limited to the blade 1175, and may include moving members for performing various purposes and functions. For example, various moving members that move linearly in the end tool may be included, including a wedge of a stapler or a moving member as such.

In addition, the cutting manipulation unit of the manipulation unit is not limited to controlling a linear movement of the blade, and may include a staple manipulation unit that performs stapling and cutting by linearly moving a moving member that moves along an axis of the connection unit (shaft) through the blade wire.

### End Tool

Hereinafter, the end tool 1100 of the surgical instrument 10 for electrocautery of FIG. 2 will be described in more detail.

FIG. 2 is a perspective view illustrating a surgical instrument for electrocautery according to an embodiment of the present disclosure. FIGS. 3 to 8 are views illustrating an end tool of the surgical instrument for electrocautery of FIG. 2.

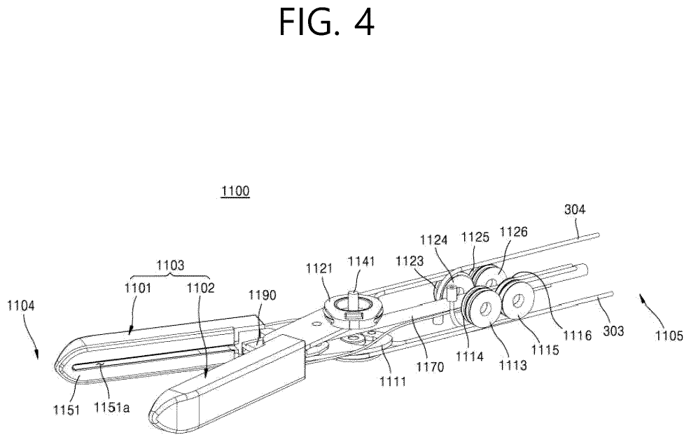

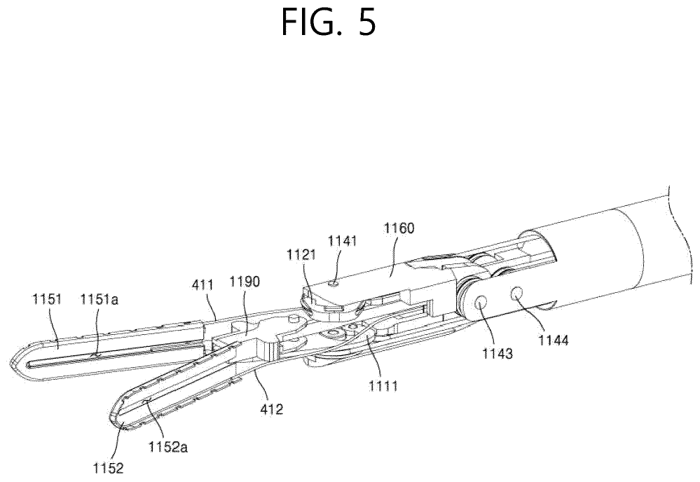

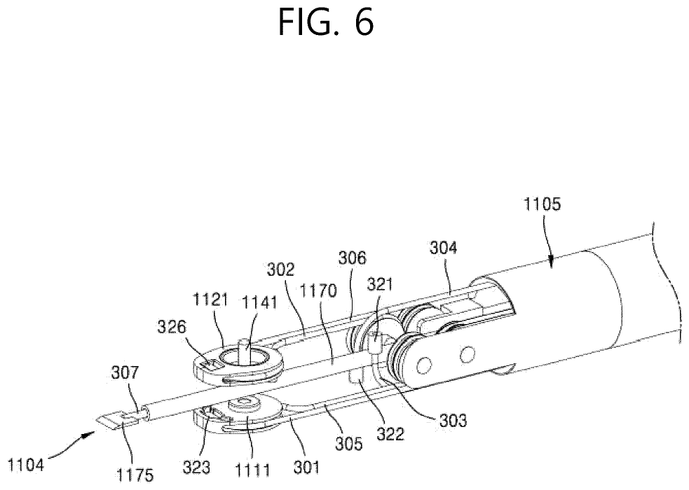

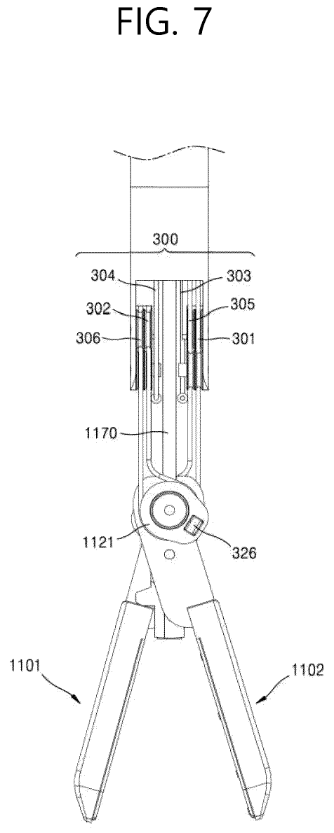

Herein, FIG. 3 illustrates a state in which an end tool hub 1160 and a pitch hub 1150 are coupled, and FIG. 4 illustrates a state in which the end tool hub 1160 and the pitch hub 1150 are removed. FIG. 5 illustrates a state in which a first jaw 1101 and a second jaw 1102 are removed, and FIG. 6 illustrates a state in which the first jaw 1101, the second jaw 1102, a pulley 1111, and a pulley 1121 are removed. FIG. 7 mainly illustrates the wires, and FIG. 14 mainly illustrates the pulleys.

With reference to FIGS. 2 to 24, the end tool 1100 of an embodiment of the present disclosure includes a pair of jaws for performing a grip motion, in other words, the first jaw 1101 and the second jaw 1102. Herein, a component encompassing each of the first jaw 1101 and the second jaw 1102 or both of the first jaw 1101 and the second jaw 1102 may be referred to as a jaw 1103.

In addition, the end tool 1100 may include a pulley 1111, a pulley 1113, a pulley 1114, a pulley 1115, and a pulley 1116 associated with the rotational motion of the first jaw 1101. In addition, the end tool 1100 may include a pulley 1121, a pulley 1123, a pulley 1124, a pulley 1125, and a pulley 1126 associated with the rotational motion of the second jaw 1102.

Herein, the drawings illustrate that the facing pulleys are formed in parallel with each other; however, the concepts of the present disclosure are not limited thereto, and each pulley may be formed in various positions and sizes suitable for the configuration of the end tool.

In addition, the end tool 1100 of an embodiment of the present disclosure may include the end tool hub 1160 and the pitch hub 1150.

A first rotation axis 1141 to be described later may penetrate and be inserted into the end tool hub 1160, and the pulley 1111 and the pulley 1121 axially coupled to the first rotation axis 1141, and at least a portion of the first jaw 1101 and the second jaw 1102 coupled thereto may be accommodated inside the end tool hub 1160. Herein, in an embodiment of the present disclosure, a wire guide unit 1168 serving as an auxiliary pulley is formed in the end tool hub 1160. In other words, a first wire guide unit 1168a and a second wire guide unit 1168b for guiding paths of the wire 305 and the wire 302 may be formed in the end tool hub 1160. The wire guide units 1268 of the end tool hub 1160 may serve as the auxiliary pulleys and change the paths of the wires, and the first wire guide unit 1168a and the second wire guide unit 1168b of the end tool hub 1260 serving as the auxiliary pulleys will be described in more detail later.

The first pitch pulley unit 1163a and a second pitch pulley portion 1163b, which serve as end tool pitch pulleys, may be formed at one end portion of the end tool hub 1160. The wire 303 and the wire 304, which are pitch wires, are coupled to the first pitch pulley unit 1163a and the second pitch pulley unit 1163b that serve as end tool pitch pulleys, and a pitch motion is performed while the end tool hub 1160 rotates about a third rotation axis 1143.

The third rotation axis 1143 and a fourth rotation axis 1144 are penetrated and inserted through the pitch hub 1150, and the pitch hub 1150 may be axially coupled to the end tool hub 1160 by the third rotation axis 1143. Accordingly, the end tool hub 1160 may be formed to be pitch-rotatable about the third rotation axis 1143 with respect to the pitch hub 1150.

In addition, the pitch hub 1150 may accommodate at least a portion of the pulley 1113, the pulley 1114, the pulley 1123, and the pulley 1124 axially coupled to the third rotation axis 1143. In addition, the pitch hub 1150 may accommodate at least a portion of the pulley 1115, the pulley 1116, the pulley 1125, and the pulley 1126 axially coupled to the fourth rotation axis 1144.

One end portion of the pitch hub 1150 is connected to the end tool hub 1160, and the other end portion of the pitch hub 1150 is connected to the connection unit 400.

Herein, the end tool 1100 of an embodiment of the present disclosure may include the first rotation axis 1141, the third rotation axis 1143, and the fourth rotation axis 1144. As described above, the first rotation axis 1141 may be penetrated and inserted through the end tool hub 1160, and the third rotation axis 1143 and the fourth rotation axis 1144 may be penetrated and inserted through the pitch hub 1150.

The first rotation axis 1141, the third rotation axis 1143, and the fourth rotation axis 1144 may be disposed sequentially from the distal end 1104 toward the proximal end 1105 of the end tool 1100. Accordingly, starting from the distal end 1104, the first rotation axis 1141 may be referred to as a first pin, the third rotation axis 1143 may be referred to as a third pin, and the fourth rotation axis 1144 may be referred to as a fourth pin.

Herein, the first rotation axis 1141 may function as an end tool jaw pulley rotation axis, the third rotation axis 1143 may function as an end tool pitch rotation axis, and the fourth rotation axis 1144 may function as an end tool pitch auxiliary rotation axis of the end tool 1100. Herein, each of the rotation axes may include two axes of a first sub-axis and a second sub-axis. Alternatively, each of the rotation axes is formed by being divided into two parts.

For example, the first rotation axis 1141 may include two axes of a first sub-axis 1141a and a second sub-axis 1141b. In addition, the third rotation axis 1143 may include two axes of a first sub-axis 1143a and a second sub-axis 1143b. In addition, the fourth rotation axis 1144 may include two axes of a first sub-axis and a second sub-axis.

Each of the rotation axes is formed by being divided into two parts as described above to allow the guide tube 1170 to be described later to pass through the end tool hub 1160 and the pitch hub 1150. In other words, the guide tube 1170 may pass between the first sub-axis and the second sub-axis of each of the rotation axes. This will be described in more detail later. Herein, the first sub-axis and the second sub-axis may be disposed on the same axis or may be disposed to be offset to a certain degree.

It is illustrated in the drawings that each of the rotation axes is formed by being divided into two parts, but the concept of the present disclosure is not limited thereto. In other words, each of the rotation axes is formed to be curved in the middle such that an escape path for the guide tube 1170 is formed.

Each of the rotation axes 1141, 1143, and 1144 may be fitted into one or more pulleys, which will be described in detail below.

The end tool 1100 may further include an actuation rotation axis 1145. In detail, the first jaw 1101 and the second jaw 1102 may be axially coupled by the actuation rotation axis 1145, and in this state, an actuation motion may be performed while the first jaw 1101 and the second jaw 1102 rotate around the actuation rotation axis 1145. Herein, the actuation rotation axis 1145 may be disposed closer to the distal end 1104 than the first rotation axis 1141 is.

Herein, in the end tool 1100 of an embodiment of the present disclosure, the first rotation axis 1141, which is a yaw rotation axis, and the actuation rotation axis 1145 are provided separately rather than as the same axis. In other words, by forming the first rotation axis 1141, which is a rotation axis of the pulley 1111/pulley 1121 that are jaw pulleys and a rotation axis of a yaw motion, and the actuation rotation axis 1145, which is a rotation axis of the second jaw 1102 with respect to the first jaw 1101 and a rotation axis of an actuation motion, to be spaced apart from each other by a certain distance, a space in which the guide tube 1170 and the blade wire 307 accommodated therein may be gently bent may be secured. The actuation rotation axis 1145 will be described in detail later.

The pulley 1111 functions as an end tool first jaw pulley, and the pulley 1121 functions as an end tool second jaw pulley. The pulley 1111 may also be referred to as a first jaw pulley, and the pulley 1121 may also be referred to as a second jaw pulley, and these two components may collectively be referred to as end tool jaw pulleys or simply jaw pulleys.

The pulley 1111 and the pulley 1121, which are end tool jaw pulleys, are formed to face each other, and are formed to be rotatable independently of each other around the first rotation axis 1141 which is an end tool jaw pulley rotation axis. In this connection, the pulley 1111 and pulley 1121 are formed to be spaced apart by a certain distance, and a blade assembly accommodation unit may be accommodated therebetween. In addition, at least a portion of a blade assembly to be described later may be disposed in the blade assembly accommodation unit. In other words, the blade assembly including the guide tube 1170 may be disposed between the pulley 1111 and the pulley 1121.

Herein, since the pulley 1111 is coupled to the first jaw 1101, when the pulley 1111 rotates around the first rotation axis 1141, the first jaw 1101 may also rotate around the first rotation axis 1141 together with the pulley 1111.

Since the pulley 1121 is connected to the second jaw 1102, when the pulley 1121 rotates around the first rotation axis 1141, the second jaw 1102 connected to the pulley 1121 may rotate around the first rotation axis 1141.

In addition, a yaw motion and an actuation motion of the end tool 1100 are performed in response to the rotation of the pulley 1111 and the pulley 1121. In other words, when the pulley 1111 and the pulley 1121 rotate in the same direction around the first rotation axis 1141, the yaw motion is performed as the first jaw 1101 and the second jaw 1102 rotate with the first rotation axis 1141 as the center of rotation. When the pulley 1111 and the pulley 1121 rotate in opposite directions around the first rotation axis 1141, the actuation motion is performed as the first jaw 1101 and the second jaw 1102 rotate around the actuation rotation axis 1145.

The pulley 1113 and the pulley 1114 function as end tool first jaw pitch main pulleys, and the pulley 1123 and the pulley 1124 function as end tool second jaw pitch main pulleys, and these two components may collectively be referred to as end tool jaw pitch main pulleys.

The pulley 1115 and the pulley 1116 function as end tool first jaw pitch sub-pulleys, and the pulley 1125 and the pulley 1126 function as end tool second jaw pitch sub-pulleys, and these two components collectively may be referred to as end tool jaw pitch sub-pulleys.

Hereinafter, components associated with the rotation of the pulley 1111 will be described.

The pulley 1113 and the pulley 1114 function as end tool first jaw pitch main pulleys. In other words, the pulley 1113 and the pulley 1114 function as main rotation pulleys for a pitch motion of the first jaw 1101. Herein, the wire 301, which is a first jaw wire, is wound around the pulley 1113, and the wire 305, which is a first jaw wire, is wound around the pulley 1114.

The pulley 1115 and the pulley 1116 function as end tool first jaw pitch sub-pulleys. In other words, the pulley 1115 and the pulley 1116 function as sub-rotation pulleys for a pitch motion of the first jaw 1101. Herein, the wire 301, which is a first jaw wire, is wound around the pulley 1115, and the wire 305, which is a first jaw wire, is wound around the pulley 1116.

Herein, the pulley 1113 and the pulley 1114 are disposed on one side of the pulley 1111 to face each other. Herein, the pulley 1113 and the pulley 1114 are formed to be rotatable independently of each other around the third rotation axis 1143 that is an end tool pitch rotation axis. In addition, the pulley 1115 and the pulley 1116 are disposed on one side of the pulley 1113 and one side of the pulley 1114, respectively, to face each other. Herein, the pulley 1115 and the pulley 1116 are formed to be rotatable independently of each other around the fourth rotation axis 1144 that is an end tool pitch auxiliary rotation axis. Here, in the drawings, it is illustrated that the pulley 1113, the pulley 1115, the pulley 1114, and the pulley 1116 are all formed to be rotatable around a Y-axis direction, but the concept of the present disclosure is not limited thereto, and the rotation axes of the respective pulleys may be formed in various directions according to configurations thereof.

The wire 301, which is a first jaw wire, is sequentially wound to make contact with at least portions of the pulley 1115, the pulley 1113, and the pulley 1111. In addition, the wire 305 connected to the wire 301 by the fastening member 323 is sequentially wound to make contact with at least portions of the pulley 1111, the first wire guide unit 1168a of the end tool hub 1160, the pulley 1114, and the pulley 1116.

In other words, the wire 301 and the wire 305, which are the first jaw wire, are sequentially wound to make contact with at least portions of the pulley 1115, the pulley 1113, the pulley 1111, the first wire guide unit 1168a of the end tool hub 1160, the pulley 1114, and the pulley 1116, and the wire 301 and the wire 305 and formed to move along the pulleys while rotating the pulleys.

Accordingly, when the wire 301 is pulled in the direction of an arrow 301 of FIG. 7, the fastening member 323 to which the wire 301 is coupled and the pulley 1111 coupled to the fastening member 323 are rotated in the counterclockwise direction. On the contrary, when the wire 305 is pulled in the direction of an arrow 305 of FIG. 7, the fastening member 323 to which the wire 305 is coupled and the pulley 1111 coupled to the fastening member 323 are rotated in the clockwise direction in the FIG. 7.

Next, components associated with the rotation of the pulley 1121 will be described.

The pulley 1123 and the pulley 1124 function as end tool second jaw pitch main pulleys. In other words, the pulley 1123 and the pulley 1124 function as main rotation pulleys for a pitch motion of the second jaw 1102. Herein, the wire 306, which is a second jaw wire, is wound around the pulley 1123, and the wire 302, which is a second jaw wire, is wound around the pulley 1124.

The pulley 1125 and the pulley 1126 function as end tool second jaw pitch sub-pulleys. In other words, the pulley 1125 and the pulley 1126 function as sub-rotation pulleys for a pitch motion of the second jaw 1102. Herein, the wire 306, which is a second jaw wire, is wound around the pulley 1125, and the wire 302, which is a second jaw wire, is wound around the pulley 1126.

Herein, the pulley 1123 and the pulley 1124 are disposed on one side of the pulley 1121 to face each other. Herein, the pulley 1123 and the pulley 1124 are formed to be rotatable independently of each other around the third rotation axis 1143 that is an end tool pitch rotation axis. In addition, the pulley 1125 and the pulley 1126 are disposed on one side of the pulley 1123 and one side of the pulley 1124, respectively, to face each other. Herein, the pulley 1125 and the pulley 1126 are formed to be rotatable independently of each other around the fourth rotation axis 1144 that is an end tool pitch auxiliary rotation axis. Herein, in the drawings, it is illustrated that all of the pulley 1123, the pulley 1125, the pulley 1124, and the pulley 1126 are formed to be rotatable around the Y-axis direction, but the concept of the present disclosure is not limited thereto, and the rotating axes of the respective pulleys may be formed in various directions according to configurations thereof.

The wire 306, which is a second jaw wire, is sequentially wound to make contact with at least portions of the pulley 1125, the pulley 1123, and the pulley 1121. In addition, the wire 302 connected to the wire 306 by the fastening member 326 is sequentially wound to make contact with at least portions of the pulley 1121, the second wire guide unit 1168b of the end tool hub 1160, the pulley 1124, and the pulley 1126.

In other words, the wire 306 and the wire 302, which are the second jaw wire, are sequentially wound to make contact with at least portions of the pulley 1125, the pulley 1123, the pulley 1121, the second wire guide unit 1168b of the end tool hub 1160, the pulley 1124, and the pulley 1126, and the wire 306 and the wire 302 are formed to move along the pulleys while rotating the pulleys.

Accordingly, when the wire 306 is pulled in the direction of an arrow 306 of FIG. 7, the fastening member 326 to which the wire 306 is coupled and the pulley 1121 coupled to the fastening member 326 are rotated in the clockwise direction in FIG. 7. On the contrary, when the wire 302 is pulled toward the arrow 302 of FIG. 7, the fastening member 326 coupled to the wire 302 and the pulley 1121 coupled to the fastening member 326 may rotate in the counterclockwise direction in FIG. 7.

Hereinafter, a pitch motion of the present disclosure will be described in more detail.

When the wire 301 is pulled in the direction of the arrow 301 of FIG. 7, and simultaneously, the wire 305 is pulled in the direction of the arrow 305 of FIG. 7 (in other words, when both strands of the first jaw wire are pulled), as shown in FIG. 6, since the wires 301 and 305 are wound around lower portions of the pulley 1113 and the pulley 1114 rotatable around the third rotation axis 1143, which is an end tool pitch rotation axis, the pulley 1111 to which the wires 301 and 305 are fixedly coupled and the end tool hub 1160 to which the pulley 1111 is coupled rotate as a whole in the counterclockwise direction around the third rotation axis 1143. As a result, the end tool 1100 may rotate downward to perform the pitch motion. In this connection, since the second jaw 1102 and the wires 302 and 306 fixedly coupled thereto are wound around upper portions of the pulley 1123 and the pulley 1124 rotatable around the third rotation axis 1143, the wires 302 and 306 are released in the opposite directions of the arrows 302 and 306, respectively.

On the contrary, when the wire 302 is pulled in the direction of the arrow 302 of FIG. 7, and simultaneously, the wire 306 is pulled in the direction of the arrow 306 of FIG. 7, as shown in FIG. 6, since the wires 302 and 306 are wound around the upper portions of the pulley 1123 and the pulley 1124 rotatable around the third rotation axis 1143, which is an end tool pitch rotation axis, the pulley 1121 to which the wires 302 and 306 are fixedly coupled and the end tool hub 1160 to which the pulley 1121 is coupled rotate as a whole in the clockwise direction around the third rotation axis 1143. As a result, the end tool 1100 may rotate upward to perform the pitch motion. In this connection, since the first jaw 1101 and the wires 301 and 305 fixedly coupled thereto are wound around lower portions of the pulley 1113 and the pulley 1114 rotatable around the third rotation axis 1143, the wires 302 and 306 are moved in the opposite directions of the arrows 301 and 305, respectively.

The end tool hub 1160 of the end tool 1100 of the surgical instrument 10 for electrocautery of an embodiment of the present disclosure may further include the first pitch pulley portion 1163a and the second pitch pulley portion 1163b serving as end tool pitch pulleys, the manipulation unit 200 may further include the pulley 231 and a pulley 232, which are manipulation unit pitch pulleys, and the power transmission unit 300 may further include the wire 303 and the wire 304 which are pitch wires.

In detail, the end tool hub 1160 including the first pitch pulley unit 1163a and the second pitch pulley unit 1163b may be formed to be rotatable around the third rotation axis 1143 that is an end tool pitch rotation axis. In addition, the wires 303 and 304 may serve to connect the first and second pitch pulley units 1163a and 1163b of the end tool 1100 and the pulleys 231 and 232 of the manipulation unit 200.

Accordingly, when the pulleys 231 and 232 of the manipulation unit 200 rotate, the rotation of the pulleys 231 and 232 is transmitted to the end tool hub 1160 of the end tool 1100 through the wires 303 and 304, causing the end tool hub 1160 to rotate as well, and as a result, the end tool 1100 performs a pitch motion while rotating.

In other words, the surgical instrument 10 for electrocautery according to an embodiment of the present disclosure includes the first and second pitch pulley units 1163a and 1163b of the end tool 1100, the pulleys 231 and 232 of the manipulation unit 200, and the wires 303 and 304 of the power transmission unit 300 in order to transmit driving force for a pitch motion, and thus, the driving force for the pitch motion of the manipulation unit 200 is more completely transmitted to the end tool 1100, thereby improving motion reliability.

### Blade Wire and Guide Tube

Hereinafter, the blade wire 307 and the guide tube 1170 of an embodiment of the present disclosure will be described in more detail.

The guide tube 1170 according to an embodiment of the present disclosure is formed to surround the blade wire 307 in a certain section, and in this connection, the blade wire 307 is movable inside the guide tube 1170. In other words, in a state in which in which the blade wire 307 is inserted into the guide tube 1170, the blade wire 307 is movable relative to the guide tube 1170.

Herein, the guide tube 1170 serves to guide the path of the blade wire 307 by preventing the blade wire 307 from being curved in an unintended direction when the blade wire 307 is pushed or pulled. A cutting motion may be smoothly performed by the guide tube 1170.

One end portion of the guide tube 1170 may be fixedly coupled to an actuation hub 1190 to be described later. Herein, the actuation hub 1190 may serve as a first coupling unit. In addition, the other end portion of the guide tube 1170 may be fixedly coupled to a second coupling portion (not shown) in the connection unit 400. Since both end portions of the guide tube 1170 are fixedly coupled to certain points (the first coupling unit and the second coupling unit) as described above, respectively, the entire length of the guide tube 1170 may remain constant. Accordingly, the length of the blade wire 307 inserted into the guide tube 1170 may also remain constant.

The guide tube 1170 according to an embodiment of the present disclosure may be formed of a flexible material and formed to be bendable. Accordingly, when the end tool 1100 performs a yaw motion around the first rotation axis 1141 or a pitch motion around the third rotation axis 1143, the guide tube 1170 may be bent while being deformed in shape corresponding thereto. In addition, when the guide tube 1170 is bent, the blade wire 307 placed therein is also bent.

Herein, although the length of the guide tube 1170 is constant, the relative position and distance of the first coupling unit (in other words, the actuation hub 1190) and the second coupling unit (not shown) may be changed as the end tool 1100 is pitch-rotated or yaw-rotated, and thus a space for the guide tube 1170 to move by the changed distance is required. To this end, a pitch slit 1164 and a yaw slit 1165 may be provided in the end tool hub 1160 to form spaces for movement of the guide tube 1170. Such a configuration of the end tool hub 1160 will be described in detail later.

As described above, the blade wire 307 is penetrated and inserted through the guide tube 1170, and the blade wire 307 is relatively movable inside the guide tube 1170 with respect to the guide tube 1170. In other words, when the blade wire 307 is pulled in a state in which the guide tube 1170 is fixed, the blade 1175 connected to the blade wire 307 is moved toward the proximal end 1105, and when the blade wire 307 is pushed, the blade 1175 connected to the blade wire 307 is moved toward the distal end 1104.

This will be described below in more detail.

The most reliable way to perform a cutting motion using the blade 1175 is by pushing and pulling the blade 1175 with the blade wire 307. In addition, in order for the blade wire 307 to push and pull the blade 1175, the guide tube 1170 that may guide the path of the blade wire 307 needs to be provided. When the guide tube 1170 does not guide the path of the blade wire 307 (in other words, does not hold the blade wire 307), a phenomenon may occur in which cutting is not performed and a middle portion of the blade wire 307 is curved even when the blade wire 307 is pushed. Accordingly, in order to reliably perform the cutting motion using the blade 1175, the blade wire 307 and the guide tube 1170 need to be essentially included.

In detail, since the blade wire 307 also needs to bend according to the pitch and yaw motions, the blade wire 307 needs to be configured of a flexible wire. However, when trying to transmit a pushing force from one end to the other end through a flexible wire, the bending causes a loss in the transmission distance, making it very difficult to transmit the force. (For example, this may be compared to the situation of pushing an object on the floor with a stick and the situation of pushing the object with a rope.) In other words, the movement distance of the side applying the load is lost due to the reduction in the path length due to the bending, and accordingly, the movement distance of the side receiving the load becomes shorter. Accordingly, in order to prevent such a loss of movement distance, the reduction in the path length needs to be prevented, and as a method for preventing this reduction, the path lengths on both sides are fixed using a guide tube 1170.

When the blade wire 307 is used to drive a cutting motion, the cutting needs be performed while pushing the blade wire 307, and in this connection, in order for the blade wire 307 to receive a force, a relatively stiff (in other words, non-bendable) wire needs to be used for the blade wire 307. However, the stiff (in other words, non-bendable) wire may have a small bendable range and may be permanently deformed when a force equal to or greater than a certain degree is applied.

In other words, in the case of a stiff (in other words, non-bendable) wire, there is a minimum radius of curvature that may be bent and spread without permanent deformation. In other words, when the wire or the guide tube is curved below a specific radius of curvature, both the wire and the guide tube may undergo permanent deformation while being bent, thereby restricting the capacity to perform cutting while moving backward and forward. Thus, it is necessary to keep the blade wire 307 curved while having a gentle curvature.

Thus, in order to prevent the blade wire 307 from being rapidly bent while passing through the pulleys, a space, in which the blade wire 307 may be gently bent, is required between the jaw 1103 (in other words, the actuation rotation axis 1145) and the end tool hub 1160 (in other words, the first rotation axis 1141 that is a yaw axis).

To this end, according to an embodiment of the present disclosure, the first rotation axis 1141, which is a yaw rotation axis, and the actuation rotation axis 1145 are separately provided, and the first rotation axis 1141 and the actuation rotation axis 1145 are spaced apart from each other by a certain distance, thereby forming a space in which the blade wire 307 and the guide tube 1170 may be gently bent.

In addition, since the blade wire 307 and the guide tube 1170 need to be connected to the blade 1175 through the end tool hub 1160, and a space in which the blade wire 307 and the guide tube 1170 is able to be bent in the end tool hub 1160 is necessary, in an embodiment of the present disclosure, 1) spaces, through which the blade wire 307/the guide tube 1170 are able to pass and simultaneously are bendable, that is, the pitch slit 1164 and the yaw slit 1165, are formed in the end tool hub 1160, 2) each of the rotation axes is formed by being divided into two parts, and 3) a pitch round unit 1166 and a yaw round unit 1167 are additionally formed to guide the bending of the blade wire 307 and the guide tube 1170.

In other words, when one end portion of the guide tube 1170 is fixed in the connection unit 400, and the other end portion thereof is moved while performing pitch and yaw motions, the guide tube 1170 is curved in a direction, in which the gentlest curvature (hereinafter, referred to as “maximum gentle curvature”) may be achieved in response to a change in a distance between both end portions thereof. As such, by achieving the maximum gentle curvature of the natural state, the motion of the blade wire 307 is smooth and the permanent deformation does not occur.

Thus, in order to secure the maximum gentle curvature, the pitch slit 1164 and the yaw slit 1165 are formed on the path of the guide tube 1170, and furthermore, the pitch round unit 1166 and the yaw round unit 1167 may be additionally formed in the end tool hub 1160. Accordingly, the guide tube 1170 may have such a shape that is the most similar to the maximum gentle curvature (although not having the maximum gentle curvature).

Hereinafter, the end tool hub 1160 will be described in more detail.

### End Tool Hub

FIG. 9 is a perspective view illustrating an end tool hub of the surgical instrument for electrocautery of FIG. 2. FIGS. 10 and 11 are cut-away perspective views of the end tool hub of FIG. 9. FIGS. 12 and 13 are perspective views illustrating the end tool hub of FIG. 9. FIG. 14 is a side view illustrating the end tool hub and the guide tube of FIG. 9. FIG. 15 is a plan view illustrating the end tool hub and the guide tube of FIG. 9.

Referring to FIGS. 9 to 15, the end tool hub 1160 includes a body unit 1161, a first jaw pulley coupling unit 1162a, a second jaw pulley coupling unit 1162b, the first pitch pulley unit 1163a, the second pitch pulley unit 1163b, the pitch slit 1164, the yaw slit 1165, the pitch round unit 1166, the yaw round unit 1167, and the wire guide unit 1168. In addition, the wire guide unit 1168 includes the first wire guide unit 1168a and the second wire guide unit 1168b.

The first jaw pulley coupling unit 1162a and the second jaw pulley coupling unit 1162b may be formed in the end tool hub 1160 at the distal end side. Herein, the first jaw pulley coupling unit 1162a and the second jaw pulley coupling unit 1162b are formed to face each other, and the pulley 1111 and the pulley 1121 are accommodated therein. Herein, the first jaw pulley coupling unit 1162a and the second jaw pulley coupling unit 1162b may be formed to be approximately parallel to a plane perpendicular to the first rotation axis 1141 that is a yaw rotation axis.

The first jaw pulley coupling unit 1162a and the second jaw pulley coupling unit 1162b are connected by the body unit 1161. In other words, the first jaw pulley coupling unit 1162a and the second jaw pulley coupling unit 1162b, which are parallel to each other, are coupled by the body unit 1161 formed in a direction approximately perpendicular to the first jaw pulley coupling unit 1162a and the second jaw pulley coupling unit 1162b, so that the first jaw pulley coupling unit 1162a, the second jaw pulley coupling unit 1162b, and the body unit 1161 form an approximately U-shape, in which the pulley 1111 and the pulley 1121 are accommodated.

In other words, the first jaw pulley coupling unit 1162a and the second jaw pulley coupling unit 1162b are formed to extend in the X-axis direction from the body unit 1161.

Herein, the pulley 1111, which is a first jaw pulley, is disposed adjacent to the first jaw pulley coupling unit 1162a of the end tool hub 1160, and the pulley 1121, which is a second jaw pulley, is disposed adjacent to the second jaw pulley coupling unit 1162b of the end tool hub 1160, and thus the yaw slit 1165 may be formed between the first jaw pulley coupling unit 1162a and the second jaw pulley coupling unit 1162b. In addition, at least a portion of the blade assembly to be described later may be disposed in the yaw slit 1165. In other words, at least a portion of the guide tube 1170 of the blade assembly may be disposed between the first jaw pulley coupling unit 1162a and the second jaw pulley coupling unit 1162b. As such, by disposing the blade assembly including the guide tube 1170 between the pulley 1111, which is a first jaw pulley, and the pulley 1121, which is a second jaw pulley, the end tool 1100 is able to perform the cutting motion using the blade 1175 in addition to the pitch and yaw motions. This will be described in more detail later.

A through hole is formed in the first jaw pulley coupling unit 1162a such that the first rotation axis 1141 passes through the first jaw pulley coupling unit 1162a and the pulley 1111 and axially couples the first jaw pulley coupling unit 1162a and the pulley 1111. In addition, a through hole is formed in the second jaw pulley coupling unit 1162b such that the first rotation axis 1141 passes through the second jaw pulley coupling unit 1162b and the pulley 1121 and axially couples the second jaw pulley coupling unit 1162b and the pulley 1121.

In this connection, as described above, the first rotation axis 1141, which is a yaw rotation axis, may be formed by being divided into two parts of the first sub-axis 1141a and the second sub-axis 1141b, and the guide tube 1170 may pass between the first sub-axis 1141a and the second sub-axis 1141b of the first rotation axis 1141.

In addition, the yaw slit 1165 may be formed between the first jaw pulley coupling unit 1162a and the second jaw pulley coupling unit 1162b. Since the yaw slit 1165 is formed in the end tool hub 1160 as described above, the guide tube 1170 may pass through the inside of the end tool hub 1160.

In other words, the first rotation axis 1141 is vertically separated without passing through the end tool hub 1160, and the yaw slit 1165 may be formed on a plane perpendicular to the first rotation axis 1141 in the vicinity of the first rotation axis 1141. Accordingly, the guide tube 1170 is movable (in other words, movable left and right) in the yaw slit 1165 while passing through the vicinity of the first rotation axis 1141.

The yaw round portion 1167 may be further formed in the body unit 1161. The yaw round unit 1167 may be formed to be rounded so as to have a predetermined curvature. In detail, when viewed from a plane perpendicular to the first rotation axis 1141 that is a yaw rotation axis, the yaw round unit 1167 may be formed to be rounded so as to have a predetermined curvature. For example, the yaw round unit 1167 may be formed in a fan shape, and may be formed along a path in which the guide tube 1170 is bent on an XY plane. The yaw round unit 1167 as described above may serve to guide the path of the guide tube 1170 when the end tool 1100 yaw rotates.

The wire guide unit 1168, which guides a path of the wire passing through the inside of the end tool hub 1160, is formed at one side of the body unit 1161. Herein, the wire guide unit 1168 includes the first wire guide unit 1168a and the second wire guide unit 1168b. Herein, the first wire guide unit 1168a may be formed on an inner side surface of the first jaw pulley coupling unit 1162a. In addition, the second wire guide unit 1168b may be formed on an inner side surface of the second jaw pulley coupling unit 1162b.

Herein, the wire guide unit 1168 may be formed in a cylindrical shape with a cross section that is approximately semi-circular. In addition, the semi-circular unit may be disposed to protrude toward the pulley 1111 and the pulley 1121. In other words, the wire guide unit 1168 is formed to protrude toward a space formed by the first jaw pulley coupling unit 1162a, the second jaw pulley coupling unit 1162b, and the body unit 1161. In other words, in the wire guide unit 1168, a region adjacent to the first jaw pulley coupling unit 1162a and the second jaw pulley coupling unit 1162b is formed to have a cross section that is curved with a predetermined curvature.

Alternatively, in other words, the wire guide unit 1168 functions as a kind of pulley member, which guides the paths of the wire 305 and the wire 302 by winding the wire 305 and the wire 302 around an outer circumferential surface thereof. However, herein, the wire guide unit 1168 is not a member that rotates around a certain axis as the original meaning pulley does, and the wire guide unit 1168 is formed to be fixed as a portion of the end tool hub 1160 and performs some similar functions of a pulley by winding a wire therearound.

Herein, the wire guide unit 1168 is illustrated in the drawing as being formed in a cylindrical shape with a cross section that is approximately semi-circular. In other words, at least a portion of the cross section of the wire guide unit 1168 on the XY plane is illustrated as having a certain arc shape. However, the concept of the present disclosure is not limited thereto, and the cross section may have a predetermined curvature like an oval or a parabola, or a corner of a polygonal column is rounded to a certain degree, so that the cross section may have various shapes and sizes suitable for guiding the paths of the wire 305 and the wire 302.

Herein, a guide groove for guiding the paths of the wire 305 and the wire 302 well may be further formed in a portion of the wire guide portion 1168, which is in contact with the wire 305 and the wire 302. The guide groove may be formed in the form of a groove recessed to a certain degree from a protruding surface of the wire guide unit 1168.

Herein, although the guide groove is illustrated in the drawing as being formed in the entire arc surface of the wire guide unit 1168, the concept of the present disclosure is not limited thereto, and the guide groove may be formed only in a portion of the arc surface of the wire guide unit 1168 as necessary.

As described above, by further forming the guide groove in the wire guide unit 1168, unnecessary friction between the wires is reduced, so that durability of the wires may be improved.

The first pitch pulley unit 1163a and the second pitch pulley unit 1163b, which serve as end tool pitch pulleys, may be formed on the end tool hub 1160 at the proximal end side. Herein, the first pitch pulley unit 1163a and the second pitch pulley unit 1163b may be formed to face each other. Herein, the first pitch pulley unit 1163a and the second pitch pulley unit 1163b may be formed to be approximately parallel to a plane perpendicular to the third rotation axis 1143, which is a pitch rotation axis.

In detail, one end portion of the end tool hub 1160 is formed in a disk shape similar to a pulley, and grooves around which a wire may be wound may be formed on an outer circumferential surface of the one end portion, thereby forming the first pitch pulley unit 1163a and the second pitch pulley unit 1163b. The wire 303 and the wire 304 described above are coupled to the first pitch pulley unit 1163a and the second pitch pulley unit 1163b, which serve as end tool pitch pulleys, and a pitch motion is performed while the end tool hub 1160 rotates around the third rotation axis 1143.

Although not illustrated in the drawings, the pitch pulley may be formed as a separate member from the end tool hub 1160 and coupled to the end tool hub 1160.

The first pitch pulley unit 1163a and the second pitch pulley unit 1163b may be connected by the body unit 1161. In other words, the first pitch pulley unit 1163a and the second pitch pulley unit 1163b, which are parallel to each other, are coupled by the body unit 1161 formed in a direction approximately perpendicular to the first pitch pulley unit 1163a and the second pitch pulley unit 1163b, and thus the first pitch pulley unit 1163a, the second pitch pulley unit 1163b, and the body unit 1161 may form an approximately U-shape.

In other words, the first pitch pulley unit 1163a and the second pitch pulley unit 1163b are formed to extend from the body unit 1161 in the X-axis direction.

A through hole is formed in the first pitch pulley unit 1163a so that the third rotation axis 1143 may penetrate the first pitch pulley unit 1163a. In addition, a through hole is formed in the second pitch pulley unit 1163b so that the third rotation axis 1143 may penetrate the second pitch pulley unit 1163b.

In this connection, as described above, the third rotation axis 1143, which is a pitch rotation axis, may be formed by being divided into two parts of the first sub-axis 1143a and the second sub-axis 1143b, and the guide tube 1170 may penetrate between the first sub-axis 1143a and the second sub-axis 1143b of the third rotation axis 1143.

The pitch slit 1164 may be formed between the first pitch pulley unit 1163a and the second pitch pulley unit 1163b. Since the pitch slit 1164 is formed in the end tool hub 1160 as described above, the guide tube 1170 may penetrate the inside of the end tool hub 1160.

In other words, the third rotation axis 1143 is horizontally separated into two parts without penetrating the end tool hub 1160, and the pitch slit 1164 may be formed on a plane perpendicular to the third rotation axis 1143 in the vicinity of the third rotation axis 1143. Accordingly, the guide tube 1170 is movable (movable up and down) in the pitch slit 1164 while penetrating the vicinity of the third rotation axis 1143.

The pitch round unit 1166 may be further formed in the body unit 1161. The pitch round unit 1166 may be formed to be rounded to have a predetermined curvature. In detail, when viewed from a plane perpendicular to the third rotation axis 1143, which is a pitch rotation axis, the pitch round unit 1166 may be formed to be rounded to have a predetermined curvature. For example, the pitch round unit 1166 may be formed in a fan shape, and formed along a path in which the guide tube 1170 is bent on the XZ plane. The pitch round unit 1166 as described above may serve to guide the path of the guide tube 1170 when the end tool 1100 pitch-rotates.

Herein, the pitch slit 1164 and the yaw slit 1165 may be formed to be connected to each other. Accordingly, the guide tube 1170 and the blade wire 307 located therein may be disposed to completely penetrate the inside of the end tool hub 1160. In addition, the blade 1175 coupled to one end portion of the blade wire 307 may linearly reciprocate inside the first jaw 1101 and the second jaw 1102.

As described above, since the blade wire 307 and the guide tube 1170 need to be connected to the blade 1175 through the end tool hub 1160, and a space in which the blade wire 307 and the guide tube 1170 are able to be bent in the end tool hub 1160 is necessary, in an embodiment of the present disclosure, 1) spaces, through which the blade wire 307/the guide tube 1170 may pass and simultaneously are bendable, that is, the pitch slit 1164 and the yaw slit 1165, are formed in the end tool hub 1160, 2) the rotation axes are formed by being divided into two parts, and 3) the pitch round unit 1166 and the yaw round unit 1167 are additionally formed to guide the bending of the blade wire 307/the guide tube 1170.

Hereinafter, the role and function of the wire guide unit 1168 will be described in more detail.

The wire guide unit 1168 may be in contact with the wire 305 and the wire 302 and may change the disposition path of the wire 305 and the wire 302 to a certain degree to serve to increase a rotation radius of each of the first jaw 1101 and the second jaw 1102.

In other words, when the auxiliary pulleys are not disposed, each of the pulley 1111, which is a first jaw pulley, and the pulley 1121, which is a second jaw pulley, may rotate up to a right angle, but in an embodiment of the present disclosure, by additionally providing the wire guide unit 1168 in the end tool hub 1160, the maximum rotation angle of each pulley may be increased.

This enables a motion in which two jaws of the end tool 1100 have to be spread apart for an actuation motion in a state in which the two jaws are yaw-rotated together by 90°. In other words, the range of yaw rotation in which an actuation motion is possible may be increased through the configuration of the wire guide unit 1168 of the end tool hub 1160. In other words, the range of yaw rotation in which an actuation motion is possible may be increased through the configuration of the wire guide unit 1168 of the end tool hub 1160.

Furthermore, by forming the wire guide unit 1168 in the end tool hub 1160, which already exists, without adding a separate structure such as an auxiliary pulley, the range of rotation may be increased without adding a component and a manufacturing process.

As described above, since there is no need to additionally dispose a separate structure for increasing the rotation angle, the number of components is decreased and the manufacturing process is simplified, and also, the length of the end tool is shortened by as much as the size of the auxiliary pulley, so that the length of the end tool is shortened during a pitch motion. Accordingly, a surgical motion may be more easily performed in a narrow space.

This will be described below in more detail.

In the end tool 1100 of the surgical instrument according to an embodiment of the present disclosure, the disposition path of the wires may be changed without a separate structure by forming the wire guide unit 1168 capable of changing the path of the wire on an inner side wall of the end tool hub 1160. As described above, as the disposition path of the wire 305 and the wire 302 is changed to a certain degree by forming the wire guide unit 1168 in the end tool hub 1160, a tangential direction of the wire 305 and the wire 302 is changed, and accordingly, rotation angles of the fastening member 323 and the fastening member 326 that couple respective wires and pulleys may be increased.

In other words, the fastening member 326 that couples the wire 302 and the pulley 1121 is rotatable until being located on a common internal tangent of the pulley 1121 and the wire guide portion 1168. Similarly, the fastening member (see 323 of FIG. 6) that couples the wire 305 and the pulley 1111 is rotatable until being located on a common internal tangent of the pulley 1111 and the wire guide unit 1168, so that a rotation angle of the fastening member (see 323 of FIG. 6) may be increased.

In other words, the wire 301 and the wire 305 wound around the pulley 1111 by the wire guide unit 1168 are disposed on one side with respect to a plane perpendicular to the Y-axis and passing through the X-axis. Simultaneously, the wire 302 and the wire 306 wound around the pulley 1121 by the wire guide unit 1168 are disposed on the other side with respect to the plane perpendicular to the Y-axis and passing through the X-axis.

In other words, the pulley 1113 and the pulley 1114 are disposed at one side with respect to the plane perpendicular to the Y-axis and passing through the X-axis, and the pulley 1123 and the pulley 1124 are disposed at the other side with respect to the plane perpendicular to the Y-axis and passing through the X-axis.

In other words, the wire 305 is located on the internal tangent of the pulley 1111 and the wire guide portion 1168, and a rotation angle of the pulley 1111 is increased due to the wire guide unit 1168. In addition, the wire 302 is located on the internal tangent of the pulley 1121 and the wire guide unit 1168, and the rotation angle of the pulley 1121 is increased due to the wire guide unit 1168.

In an embodiment of the present disclosure in which an auxiliary pulley is not formed and the wire guide unit 1168 capable of changing the path of a wire is formed on an inner side wall of the end tool hub 1160, the length of the end tool of the surgical instrument may be shortened as compared to the surgical instrument of an embodiment in which a separate auxiliary pulley is formed. Since the length of the end tool is shortened as described above, a surgical operator may easily manipulate a surgical instrument, and a side effect of surgery may be reduced when the surgery is performed in a narrow surgical space in the human body.

According to an embodiment of the present disclosure as described above, the rotation radii of the pulley 1111, which is a first jaw pulley, and the pulley 1121, which is a second jaw pulley, increase, so that a yaw motion range in which a normal opening/closing actuation motion and a normal cutting motion may be performed may be increased.

### Actuation Hub

FIG. 16A is a perspective view illustrating an actuation hub of the surgical instrument for electrocautery of FIG. 2 and FIG. 9. FIG. 16B is a cut-away perspective view illustrating an actuation hub of the surgical instrument for electrocautery of FIG. 2 and FIG. 9. FIG. 17 is a view illustrating a state in which the guide tube, the blade wire, and the blade are mounted on the actuation hub illustrated in the cut-away perspective view of FIG. 16B. FIG. 18 is an exploded perspective view illustrating the end tool of the surgical instrument for electrocautery of FIG. 2.

Referring to FIGS. 16A to 18, the actuation hub 1190 may be formed in the form of a box having a hollow therein. In addition, the actuation hub 1190 is coupled to each of the first jaw 1101 and the second jaw 1102. In detail, the actuation hub 1190 is axially coupled to the first jaw 1101 by a first actuation rotation axis 1145a. In addition, the actuation hub 1190 is axially coupled to the second jaw 1102 by a second actuation rotation axis 1145b. In this connection, the first actuation rotation axis 1145a and the second actuation rotation axis 1145b may be disposed on the same line in a Z-axis direction.

In addition, a tube seating unit 1190a may be formed inside the actuation hub 1190, and one end portion of the guide tube 1170 may be fixedly coupled to the tube seating unit 1190a. A blade accommodation unit 1190b may be formed inside the actuation hub 1190, and the blade 1175 may be accommodated in the blade accommodation unit 1190b.

In addition, a wire through-hole 1190c may be formed between the tube seating unit 1190a and the blade accommodation unit 1190b inside the actuation hub 1190.

In other words, the tube seating unit 1190a, the wire through-hole 1190c, and the blade accommodation unit 1190b are sequentially formed inside the actuation hub 1190, and the blade wire 307 may penetrate the inside of the actuation hub 1190 to be connected to the blade 1175.

As described above, by providing the actuation hub 1190 to which the guide tube 1170 is coupled between the first jaw 1101 and the second jaw 1102, the guide tube 1170 may not be curved, or the angle at which the guide tube 1170 is curved may be reduced, even when the first jaw 1101 or the second jaw 1102 rotates around the first rotation axis 1141 or the actuation rotation axis 1145.

In detail, in a case in which the guide tube 1170 is directly coupled to the first jaw 1101 or the second jaw 1102, when the first jaw 1101 or the second jaw 1102 rotates, one end portion of the guide tube 1170 also rotates together with the first jaw 1101 or the second jaw 1102, causing the guide tube 1170 to be curved.

On the other hand, in a case in which the guide tube 1170 is coupled to the actuation hub 1190, which is independent of the rotation of the jaw 1103, as in an embodiment of the present disclosure, even when the first jaw 1101 or the second jaw 1102 rotates, the guide tube 1170 may not be curved, or the angle at which the guide tube 1170 is curved may be reduced even when the guide tube 1170 is curved.

In other words, by changing the direct connection between the guide tube 1170 and the jaw 1103 by the actuation hub 1190 to an indirect connection, the degree to which the guide tube 1170 is curved by the rotation of the jaw 1103 may be reduced.

### First and Second Jaws and Actuation Motion

Hereinafter, a coupling structure of the first jaw 1101 and the second jaw 1102 of the end tool 1100 of the surgical instrument 10 of FIG. 2 will be described in more detail.

Referring to FIGS. 19 to 24 and the like, the first jaw 1101 includes a movable coupling hole 1101c, a jaw pulley coupling hole 1101d, and an axis penetration unit 1101e.

The first jaw 1101 is formed entirely in an elongated bar shape, and formed to be rotatable together with the pulley 1111 by being coupled to the pulley 1111 at one end portion thereof.

The movable coupling hole 1101c, the jaw pulley coupling hole 1101d, and the axis penetration unit 1101e may be formed in the first jaw 1101 at a side coupled to the pulley 1111, that is, at the proximal end side.

Herein, the movable coupling hole 1101c may be formed to have a predetermined curvature, and may be formed in an approximately elliptical shape. An axis coupling unit 1111a of the pulley 1111, which will be described later, may be fitted into the movable coupling hole 1101c. Herein, a short radius of the movable coupling hole 1101c may be formed to be substantially the same as or slightly greater than a radius of the axis coupling unit 1111a. A long radius of the movable coupling hole 1101c may be formed to be greater than the radius of the axis coupling unit 1111a. Thus, in a state in which the axis coupling unit 1111a of the pulley 1111 is fitted into the movable coupling hole 1101c of the first jaw 1101, the axis coupling unit 1111a is movable to a certain degree in the movable coupling hole 1101c. This will be described in more detail below.

The jaw pulley coupling hole 1101d is formed in the form of a cylindrical hole, and a jaw coupling unit 1111b of the pulley 1111, which will be described later, may be fitted into the jaw pulley coupling hole 1101d. Herein, a radius of the jaw pulley coupling hole 1101d may be formed to be substantially the same as or slightly greater than a radius of the jaw coupling unit 1111b. Thus, the jaw coupling unit 1111b of the pulley 1111 may be formed to be rotatably coupled to the jaw pulley coupling hole 1101d of the first jaw 1101. This will be described in more detail below.

The axis penetration unit 1101e may be formed in the first jaw 1101 at the distal end side relative to the movable coupling hole 1101c and the jaw pulley coupling hole 1101d. The axis penetration unit 1101e may be formed in the form of a hole, and the actuation rotation axis 1145 may be penetrated and inserted through the axis penetration unit 1101e.

The second jaw 1102 includes a movable coupling hole 1102c, a jaw pulley coupling hole 1102d, and an axis penetration unit 1102e.

The second jaw 1102 is formed entirely in an elongated bar shape, and formed to be rotatable together with the pulley 1121 by being coupled to the pulley 1121 at one end portion thereof.

The movable coupling hole 1102c, the jaw pulley coupling hole 1102d, and the axis penetration unit 1102e may be formed in the second jaw 1102 at a side coupled to the pulley 1111, that is, at the proximal end side.

Herein, the movable coupling hole 1102c may be formed to have a predetermined curvature, and may be formed in an approximately elliptical shape. An axis coupling unit 1121a of the pulley 1121, which will be described later, may be fitted into the movable coupling hole 1102c. Herein, a short radius of the movable coupling hole 1102c may be formed to be substantially the same as or slightly greater than a radius of the axis coupling unit 1121a. A long radius of the movable coupling hole 1102c may be formed to be greater than the radius of the axis coupling unit 1121a. Thus, in a state in which the axis coupling unit 1121a of the pulley 1121 is fitted into the movable coupling hole 1102c of the second jaw 1102, the axis coupling unit 1121a is movable to a certain degree in the movable coupling hole 1102c. This will be described in more detail below.

The jaw pulley coupling hole 1102d is formed in the form of a cylindrical hole, and a jaw coupling unit 1121b of the pulley 1121, which will be described later, may be fitted into the jaw pulley coupling hole 1102d. Herein, a radius of the jaw pulley coupling hole 1102d may be formed to be substantially the same as or slightly greater than a radius of the jaw coupling unit 1121b. Thus, the jaw coupling unit 1121b of the pulley 1121 may be formed to be rotatably coupled to the jaw pulley coupling hole 1102d of the second jaw 1102. This will be described in more detail below.

The axis penetration unit 1102e may be formed in the second jaw 1102 at the distal end side relative to the movable coupling hole 1102c and the jaw pulley coupling hole 1102d. The axis penetration unit 1102e may be formed in the form of a hole, and the actuation rotation axis 1145 may be penetrated and inserted through the axis penetration unit 1102e.

The pulley 1111, which is a first jaw pulley, may include the axis coupling unit 1111a and the jaw coupling unit 1111b. The pulley 1111 is formed entirely in the form of a rotatable disk, and the axis coupling unit 1111a and the jaw coupling unit 1111b may be formed to protrude to a certain degree from one surface of the pulley 1111. As described above, the axis coupling unit 1111a of the pulley 1111 may be fitted into the movable coupling hole 1101c of the first jaw 1101, and the jaw coupling unit 1111b of the pulley 1111 may be fitted into the jaw pulley coupling hole 1101d of the first jaw 1101. The pulley 1111 may be formed to be rotatable with the first rotation axis 1141, which is an end tool jaw pulley rotation axis, as the center of rotation.

The pulley 1121, which is a second jaw pulley, may include the axis coupling unit 1121a and the jaw coupling portion 1121b. The pulley 1121 is formed entirely in the form of a rotatable disk, and the axis coupling unit 1121a and the jaw coupling unit 1121b may be formed to protrude to a certain degree from one surface of the pulley 1121. As described above, the axis coupling unit 1112a of the pulley 1112 may be inserted into the movable coupling hole 1102c of the second jaw 1102, and the jaw coupling unit 1112b of the pulley 1112 may be inserted into the jaw pulley coupling hole 1102d of the second jaw 1102. The pulley 1121 may be formed to be rotatable with the first rotation axis 1141, which is an end tool jaw pulley rotation axis, as the center of rotation.

The coupling relationship between the components described above is as follows.

The first rotation axis 1141, which is an end tool jaw pulley rotation axis, is sequentially penetrated and inserted through the axis coupling unit 1111a of the pulley 1111, the movable coupling hole 1101c of the first jaw 1101, the movable coupling hole 1102c of the second jaw 1102, and the axis coupling unit 1121a of the pulley 1121.

The first actuation rotation axis 1145a is sequentially penetrated and inserted through the axis penetration unit 1101e of the first jaw 1101 and the actuation hub 1190. The second actuation rotation axis 1145b is sequentially penetrated and inserted through the axis penetration unit 1102e of the second jaw 1102 and the actuation hub 1190.

The axis coupling unit 1111a of the pulley 1111 is fitted into the movable coupling hole 1101c of the first jaw 1101, and the jaw coupling unit 1111b of the pulley 1111 is fitted into the jaw pulley coupling hole 1101d of the first jaw 1101.

In this connection, the jaw pulley coupling hole 1101d of the first jaw 1101 and the jaw coupling unit 1111b of the pulley 1111 are axially coupled to each other so as to be rotatable, and the movable coupling hole 1101c of the first jaw 1101 and the axis coupling unit 1111a of the pulley 1111 are movably coupled to each other (herein, “movably coupled” means that the axis coupling unit 1111a of the pulley 1111 is coupled so as to be movable to a certain degree in the movable coupling hole 1101c of the first jaw 1101).

The axis coupling unit 1121a of the pulley 1121 is fitted into the movable coupling hole 1102c of the second jaw 1102, and the jaw coupling unit 1121b of the pulley 1121 is fitted into the jaw pulley coupling hole 1102d of the second jaw 1102.

In this connection, the jaw pulley coupling hole 1102d of the second jaw 1101 and the jaw coupling unit 1121b of the pulley 1121 are axially coupled to each other to be rotatable, and the movable coupling hole 1102c of the second jaw 1102 and the axis coupling unit 1121a of the pulley 1121 are movably coupled to each other.

Herein, the pulley 1111 and the pulley 1121 rotate around the first rotation axis 1141, which is an end tool jaw pulley rotation axis. The first jaw 1101 and the second jaw 1102 rotate around the actuation rotation axis 1145. In other words, the pulley 1111 and the first jaw 1101 have different axes of rotation. Similarly, the pulley 1121 and the second jaw 1102 have different axes of rotation.

In other words, the rotation angle of the first jaw 1101 is limited to a certain degree by the movable coupling hole 1101c, but the first jaw 1101 essentially rotates around the actuation rotation axis 1145, which is a jaw rotation axis. Similarly, the rotation angle of the second jaw 1102 is limited to a certain degree by the movable coupling hole 1102c, but the second jaw 1102 essentially rotates around the actuation rotation axis 1145, which is a jaw rotation axis.

Amplification of a grip force due to the coupling relationship between the above-described components will be described.

In the surgical instrument 110 according to an embodiment of the present disclosure, the coupling structure of the first jaw 1101 and the second jaw 1102 forms an X-shaped structure, and thus, when the first jaw 1101 and the second jaw 1102 rotate in a direction of approaching each other (in other words, when the first jaw 1101 and the second jaw 1102 are closed), the grip force is greater in a direction in which the first jaw 1101 and the second jaw 1102 are closed. This will be described below in more detail.

As described above, in motions of the first jaw 1101 and the second jaw 1102 being opened and closed, there are two axes that serve as the centers of rotation for the first jaw 1101 and the second jaw 1102. In other words, the first jaw 1101 and the second jaw 1102 perform the opening and closing motion around two axes including the first rotation axis 1141 and the actuation rotation axis 1145. In this connection, the centers of rotation of the first jaw 1101 and the second jaw 1102 become the actuation rotation axis 1145, and the centers of rotation of rotation of the pulley 1111 and the pulley 1121 become the first rotation axis 1141. In this connection, the first rotation axis 1141 is an axis whose position is relatively fixed, and the actuation rotation axis 1145 is an axis whose position is relatively moved linearly. In other words, when the pulley 1111 and the pulley 1121 rotate in a state in which the position of the first rotation axis 1141 is fixed, the first jaw 1101 and the second jaw 1102 are opened/closed while the actuation rotation axis 1145, which is a rotation axis of the first jaw 1101 and the second jaw 1102, is moved backward and forward. This will be described below in more detail.

In FIG. 23A, r1 is a distance from the jaw coupling unit 1121b of the pulley 1121 to the axis coupling unit 1121a, and a length thereof is constant. Thus, a distance from the first rotation axis 1141 inserted into the axis coupling unit 1121a to the jaw coupling unit 1121b is also constant as r1.

Meanwhile, r2 of FIG. 23A is a distance from the jaw pulley coupling hole 1102d of the second jaw 1102 to the axis penetration unit 1102e, and a length thereof is constant. Thus, a distance from the jaw coupling unit 1121b of the pulley 1121 inserted into the jaw pulley coupling hole 1102d to the rotation axis 1145 inserted into the axis penetration unit 1102e is also constant as r2.

In other words, the lengths of r1 and r2 remain constant. Accordingly, when the pulley 1111 and the pulley 1121 rotate in the directions of an arrow B1 of FIG. 22B and an arrow B2 of FIG. 23B, respectively, around the first rotation axis 1141 to perform a closing motion, the first jaw 1101 and the second jaw 1102 rotate around the actuation rotation axis 1145 as the angle between r1 and r2 changes while the lengths of r1 and r2 remain constant, and in this connection, the actuation rotation axis 1145 itself is also linearly moved (in other words, is moved forward/backward) by as much as an arrow C1 of FIG. 22B and an arrow C2 of FIG. 23B.

In other words, assuming that the position of the first rotation axis 1141, which is an end tool jaw pulley rotation axis, is fixed, when the first jaw 1101 and the second jaw 1102 are closed, a force is applied in a direction in which the actuation rotation axis 1145, which is a jaw rotation axis, is moved forward (in other words, toward the distal end), and thus the grip force in the direction in which the first jaw 1101 and the second jaw 1102 are closed becomes larger.

In other words, since the lengths of r1 and r2 remain constant when the second jaw 1102 rotates around the actuation rotation axis 1145, when the pulley 1121 rotates around the first rotation axis 1141, the angle between r1 and r2 changes while the lengths of r1 and r2 remain constant. In other words, θ2, which is the angle between r1 and r2 in a state in which the second jaw 1102 is open as shown in FIG. 23A, is greater than θ1, which is the angle between r1 and r2 in a state in which the second jaw 1102 is closed as shown in FIG. 23B.

Thus, when the second jaw 1102 rotates from the open state to the close state, the angle between r1 and r2 changes, and a force is applied in a direction in which the actuation rotation axis 1145 is moved forward.

In this connection, since the first rotation axis 1141 is an axis whose position is relatively fixed, the actuation rotation axis 1145 is moved forward in the direction of the arrow C1 of FIG. 22B and the direction of the arrow C2 of FIG. 23B, and the grip force is further increased in a direction in which the second jaw 1102 is closed.

In other words, when the pulley 1111 and the pulley 1121 rotate around the first rotation axis 1141, which is an axis whose relative position is fixed, the angle θ between r1 and r2 changes while the distance between r1 and r2 remains constant. In addition, when the angle θ changes as described above, the first jaw 1101 and the second jaw 1102 push or pull the actuation rotation axis 1145, and thus the actuation rotation axis 1145 is moved forward or backward. In this connection, when the first jaw 1101 and the second jaw 1102 are rotated in the direction of closing, the grip force is further increased as the actuation rotation axis 1145 is moved forward in the directions of the arrow C1 of FIG. 22B and the arrow C2 of FIG. 23B. On the contrary, when the first jaw 1101 and the second jaw 1102 are rotated in the direction of opening, the actuation rotation axis 1145 is moved backward in directions opposite to the arrow C1 of FIG. 22B and the arrow C2 of FIG. 23B.

With this configuration, the grip force becomes stronger when the first jaw 1101 and the second jaw 1102 are closed, thereby enabling a surgical operator to perform the actuation motion powerfully even with a small force.

Components Associated with Cautery and Cutting

Subsequently, referring to FIGS. 2 to 24 and the like, the end tool 1100 of an embodiment of the present disclosure may include the first jaw 1101, the second jaw 1102, a first electrode 1151, a second electrode 1152, the guide tube 1170, and the blade 1175 in order to perform cauterizing and cutting motions.

Herein, components related to the driving of the blade, such as the guide tube 1170 and the blade 1175, may be collectively referred to as a blade assembly. In an embodiment of the present disclosure, by disposing the blade assembly including the guide tube 1170 and the blade 1175 between the pulley 1111, which is a first jaw pulley, and the pulley 1121, which a second jaw pulley, the end tool 1100 is able to perform the cutting motion using the blade 1175 in addition to the pitch and yaw motions. This will be described in more detail.

As described above, the first jaw 1101 is connected to the first jaw pulley 1111 and rotates around the first rotation axis 1141 together with the first jaw pulley 1111 when the first jaw pulley 1111 rotates around the first rotation axis 1141.

The first electrode 1151 may be formed on a surface of the first jaw 1101 facing the second jaw 1102. In addition, the second electrode 1152 may be formed on a surface of the second jaw 1102 facing the first jaw 1101.

In this connection, a slit 1151a may be formed in the first electrode 1151, and the blade 1175 may move along the slit 1151a. In addition, a slit 1152a may be formed in the second electrode 1152, and the blade 1175 may move along the slit 1152a.

Although not illustrated in the drawings, a spacer (not shown) may be formed between the first jaw 1101 and the first electrode 1151, and a spacer (not shown) may be formed between the second jaw 1102 and the second electrode 1152. The spacer (not shown) and the spacer (not shown) may include an insulating material such as ceramic. Alternatively, the first jaw 1101 and the second jaw 1102 may themselves be made of a nonconductor such that the first electrode 1151 and the second electrode 1152 may be maintained to be insulated from each other without a separate insulator until the first electrode 1151 and the second electrode 1152 are in contact with each other.

Although not illustrated in the drawings, one or more sensors (not shown) may be further formed on at least one of the first jaw 1101 or the second jaw 1102. The sensor (not shown) may be formed to measure at least some of current, voltage, resistance, impedance, and temperature during the cautery by locating tissue between the first jaw 1101 and the second jaw 1102 and passing a current through the first electrode 1151 and the second electrode 1152.

Alternatively, instead of providing a separate sensor, monitoring and controlling of at least some of current, voltage, resistance, impedance, and temperature may be directly performed by a generator (not shown) which supplies power to the electrodes.

An edge unit formed sharply and configured to cut tissue may be formed in one region of the blade 1175. The tissue disposed between the first jaw 1101 and the second jaw 1102 may be cut as at least a portion of the blade 1175 moves between the distal end 1104 and the proximal end 1105 of the end tool 1100.

Herein, the guide tube 1170 and the blade 1175 disposed between the pulley 1111 and the pulley 1121 are provided in the end tool 1100 of the surgical instrument 10 for electrocautery according to an embodiment of the present disclosure. In addition, by providing the guide tube 1170 and the blade 1175, a multi-joint/multi-degree-of-freedom surgical instrument capable of pitch/yaw/actuation motions may also perform cauterizing and cutting motions. This will be described below in more detail.

Thus far, various types of surgical instruments for electrocautery have been developed. Among the various types of surgical instruments for electrocautery, a blood vessel sealing device called “Vessel Sealer” has a sensing function added to the existing bipolar cautery method, so that power of different polarities may be supplied to two electrodes, and after denaturing a vessel with the heat generated therefrom for hemostasis, the stanched part may be cut with a blade. In this connection, the impedance of the tissue (or blood vessel) while the current is flowing is measured to determine whether the cauterization is completed, and when the cauterization is completed, the current supply is automatically stopped, and the tissue is cut with the blade.

In the case of such a bipolar-type blood vessel resection device, it is essential to have a blade to cut the tissue after cauterization, and the end tool needs to be additionally equipped with a mechanism for facilitating a linear reciprocating motion of the blade, and thus joint movements such as pitch/yaw movements are not possible in most cases.

There have been attempts to implement joint movements using flexion joints with multiple nodes connected in the bipolar-type blood vessel resection device, but in this connection, a rotation angle is limited and it is difficult to achieve accurate motion control of the end tool.

In another way, in the case of a method that utilizes vibration of ultrasonic waves to perform hemostasis and cutting, it is not feasible to provide joints due to the physical properties of ultrasonic waves.

To address these issues, the end tool 1100 of the surgical instrument 10 for electrocautery according to an embodiment of the present disclosure includes the guide tube 1170 disposed between the pulley 1111 and the pulley 1121, and the blade 1175 that moves between a first position and a second position in response to the movement of the blade wire 307 disposed inside the guide tube 1170. In addition, by providing the guide tube 1170 and the blade 1175 as described above, pitch/yaw/actuation motions may also be performed using a pulley/wire in a bipolar-type surgical instrument for cauterizing and cutting tissue.

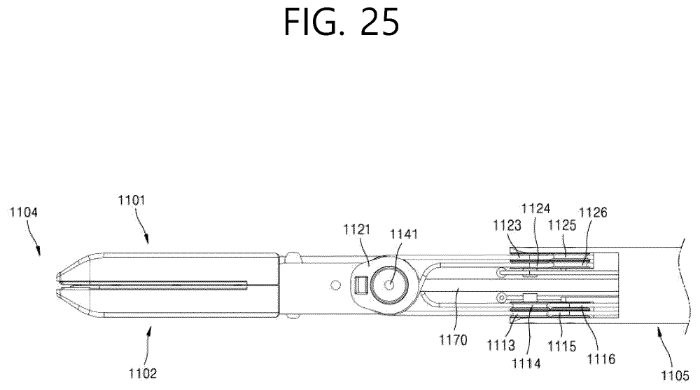

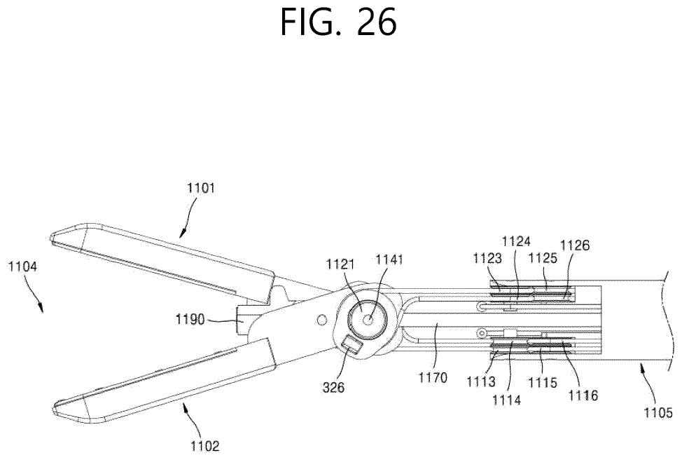

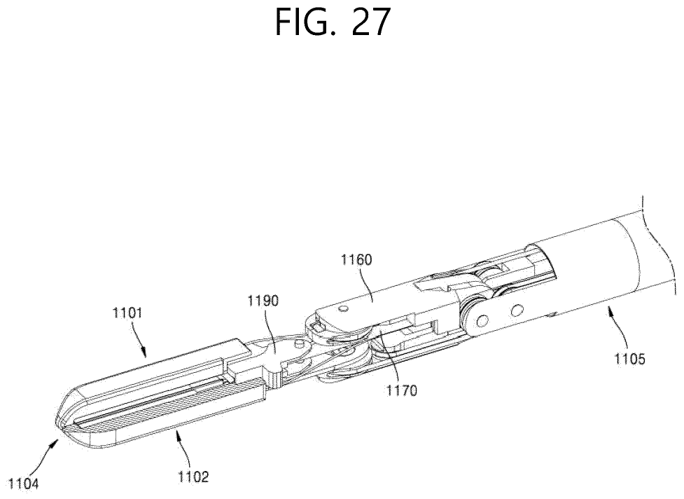

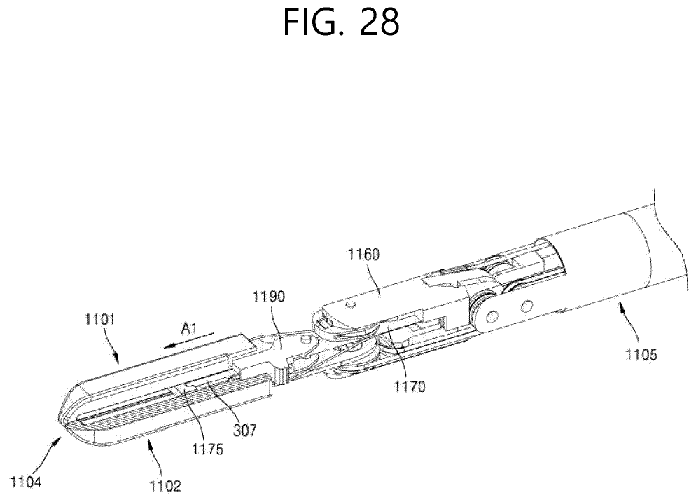

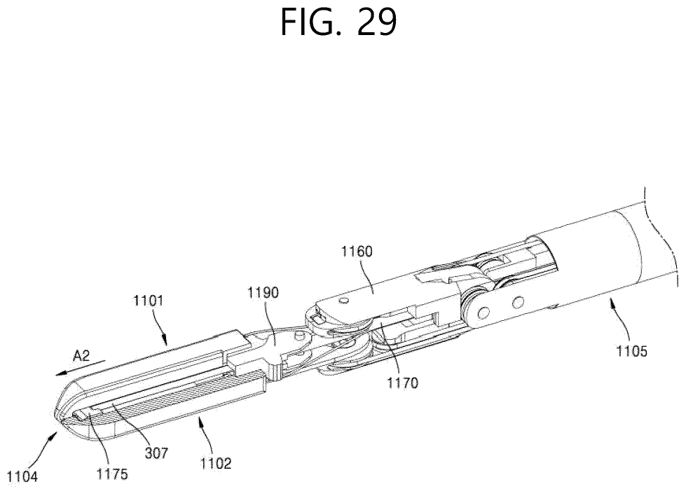

FIG. 25 is a view illustrating a state in which the end tool of the surgical instrument for electrocautery of FIG. 2 is closed, and FIG. 26 is a view illustrating a state in which the end tool of the surgical instrument for electrocautery of FIG. 2 is opened. In addition, FIG. 27 is a view illustrating a state in which the blade wire 307 and the blade 1175 are located at a first position, FIG. 28 is a view illustrating a state in which the blade wire 307 and the blade 1175 are located at a second position, and FIG. 29 is a view illustrating a state in which the blade wire 307 and the blade 1175 are located at a third position.

Referring to FIGS. 25 to 29, the tissue between the first jaw 1101 and the second jaw 1102 is cut as the cutting motion of FIGS. 27 to 29 is performed in a state in which the first jaw 1101 and the second jaw 1102 are closed as shown in FIG. 25.

Herein, the first position illustrated in FIG. 27 may be defined as a state in which the blade 1175 is drawn in toward the proximal end 1105 of the end tool 1100 as much as possible.

Alternatively, the first position may be defined as a state in which the blade 1175 is located adjacent to the pulley 1111/pulley 1121.

The third position illustrated in FIG. 29 may be defined as a state in which the blade 1175 is withdrawn toward the distal end 1104 of the end tool 1100 as much as possible. Alternatively, the third position may be defined as a state in which the blade 1175 is spaced away from the pulley 1111/pulley 1121 as much as possible.

First, as shown in FIG. 26, a tissue to be cut is located between the first jaw 1101 and the second jaw 1102 in a state in which the first jaw 1101 and the second jaw 1102 are opened, and then an actuation motion is performed to close the first jaw 1101 and the second jaw 1102 as shown in FIG. 25.

Next, as shown in FIG. 27, in a state in which the blade wire 307 and the blade 1175 are located at the first position, currents of different polarities are applied to the first electrode 1151 and the second electrode 1152 to cauterize the tissue between the first jaw 1101 and the second jaw 1102. In this connection, a generator (not shown) configured to supply power to the electrodes may itself perform monitoring of at least some of current, voltage, resistance, impedance, and temperature, and may stop supplying power when the cauterization is completed.

In the state in which the cautery is completed as described above, when the blade wire 307 moves sequentially in the directions of an arrow A1 of FIG. 17 and an arrow A2 of FIG. 29, the blade 1175 coupled to the blade wire 307 moves from the first position at the proximal end 1105 of the end tool 1100 toward the third position at the distal end 1104 of the end tool 1100, reaching the positions in FIGS. 28 and 29 in turn.

As such, the blade 1175 cuts the tissue between the first jaw 1101 and the second jaw 1102 while moving in the X-axis direction.

However, it is to be understood that the linear motion of the blade 1175 herein does not mean a motion in a completely straight line, but rather means a motion of the blade 1175 to the extent that the blade 1175 is able to cut the tissue while achieving a linear motion when viewed as a whole, even though the motion is not in a completely straight line, for example, the middle part of the straight line is bent by a certain angle or there is a section having a gentle curvature in a certain section.

In this state, when the blade wire 307 is pulled in the opposite direction, the blade 1175 coupled to the blade wire 307 also returns to the first position.

According to an embodiment of the present disclosure, the multi-joint/multi-degree-of-freedom surgical instrument capable of pitch/yaw/actuation motions may also perform cauterizing and cutting motions.

### Pitch, Yaw, and Cutting Motions of End Tool

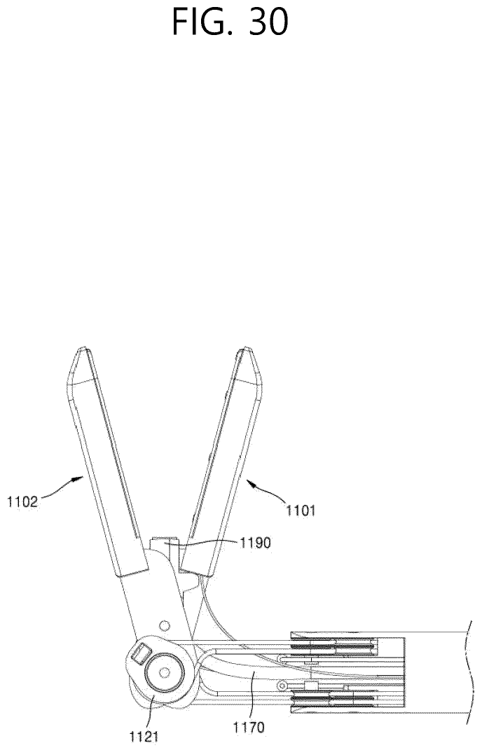

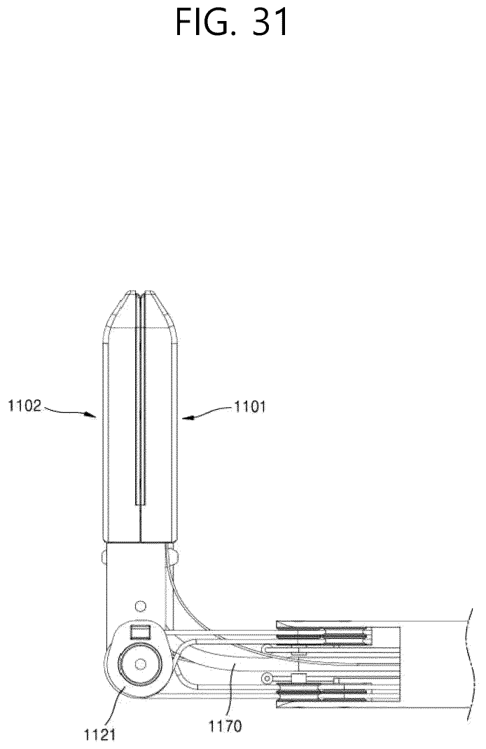

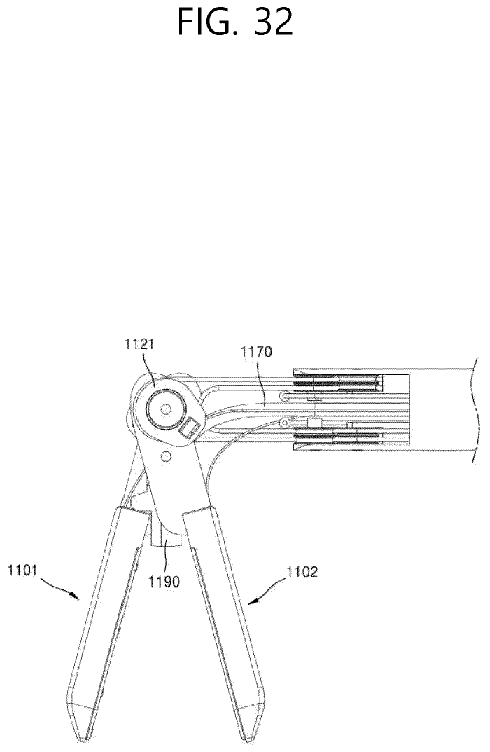

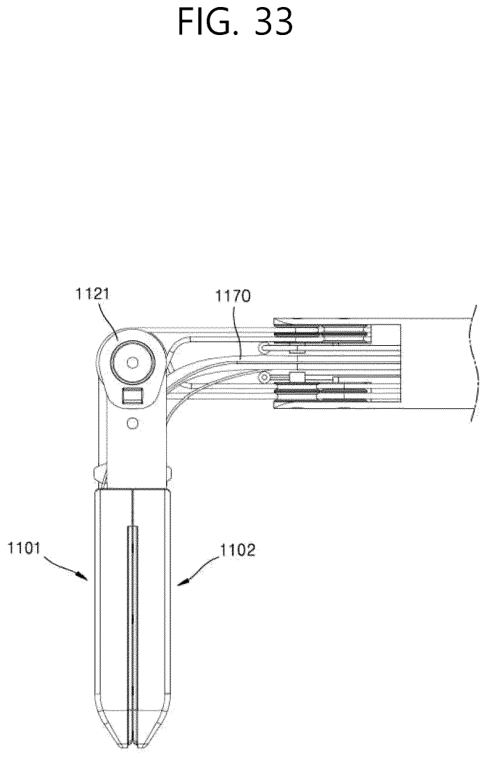

FIGS. 30 and 31 are views illustrating a process of performing an opening and closing motion in a state in which the end tool of the surgical instrument for electrocautery of FIG. 2 is yaw-rotated by +90°. In addition, FIGS. 32 and 33 are views illustrating a process of performing an opening and closing motion in a state in which the end tool of the surgical instrument for electrocautery of FIG. 2 is yaw-rotated by −90°.

As illustrated in FIGS. 30 to 33, the end tool of the surgical instrument for electrocautery according to an embodiment of the present disclosure is formed to be able to normally perform an opening and closing motion, that is, an actuation motion even in a state in which the jaws are yaw-rotated by +90° or −90°.

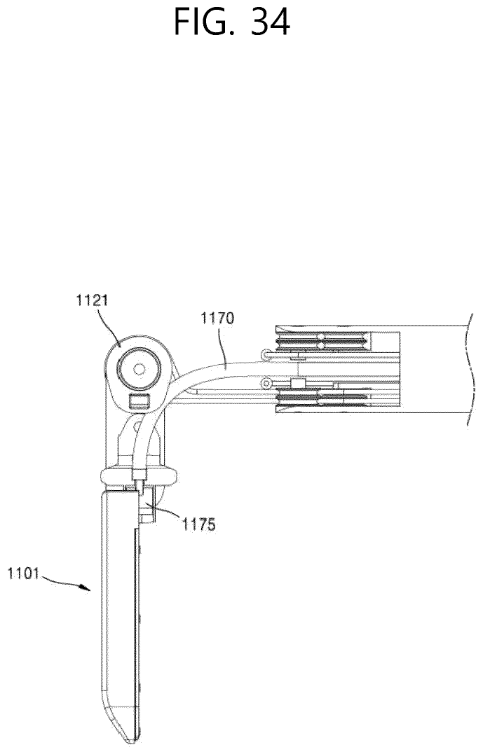

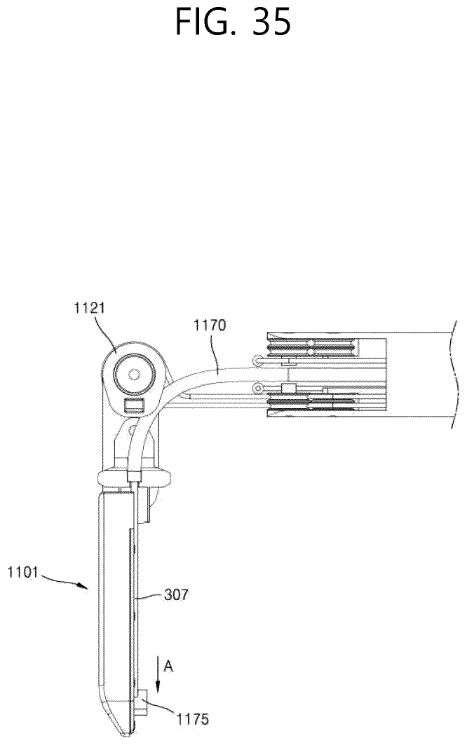

FIGS. 34 and 35 are views illustrating a path of the guide tube and a movement path of the blade during a cutting motion in a state in which the end tool of the surgical instrument for electrocautery of FIG. 2 is yaw-rotated.

As illustrated in FIGS. 34 and 35, the end tool of the surgical instrument for electrocautery according to an embodiment of the present disclosure is formed to be able to normally perform a cutting motion even in a state in which the jaws are yaw-rotated by +90°.

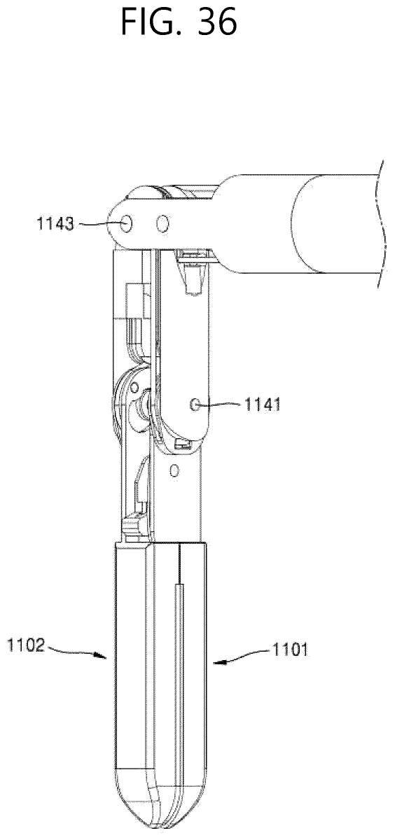

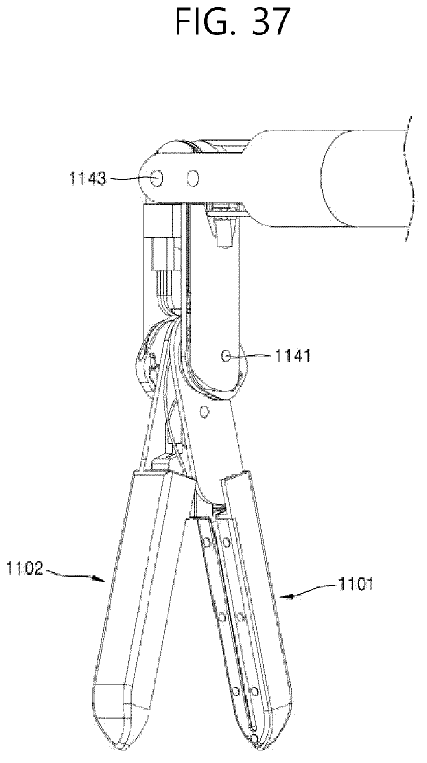

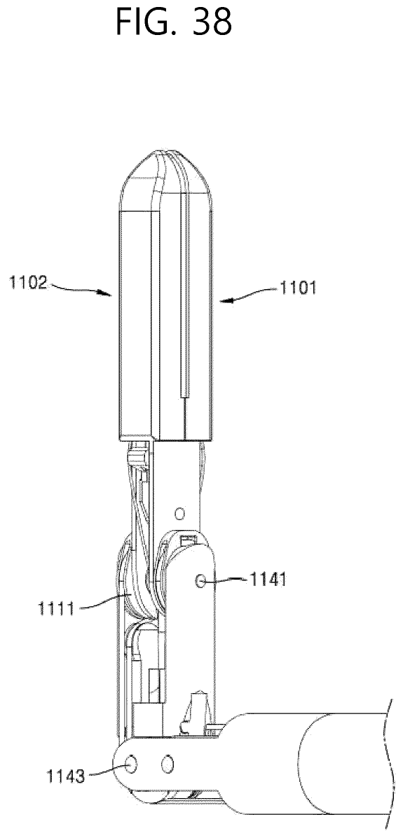

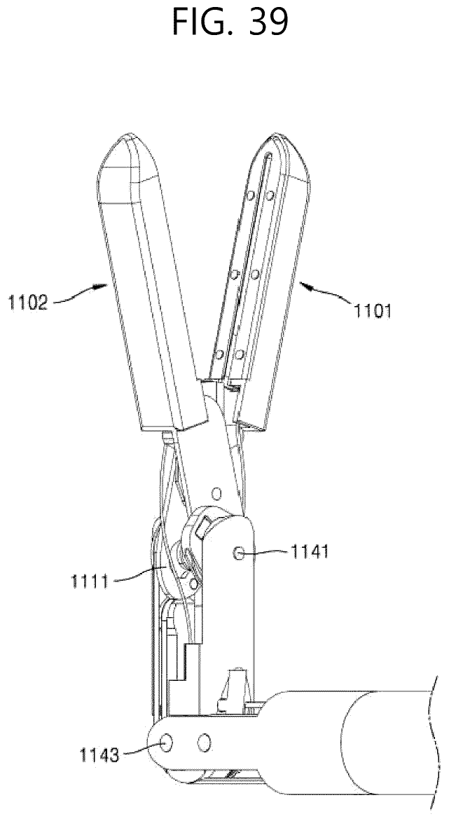

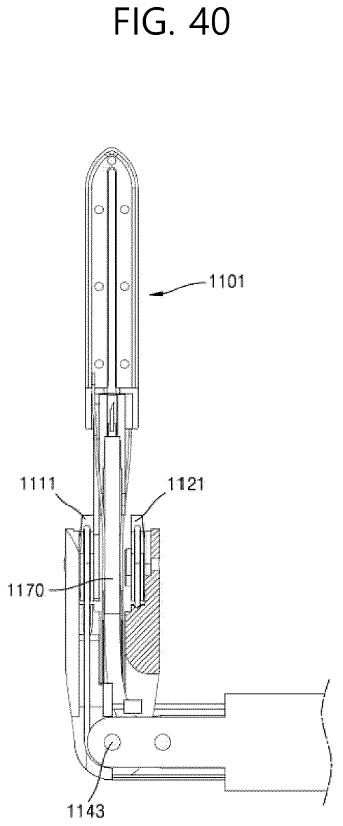

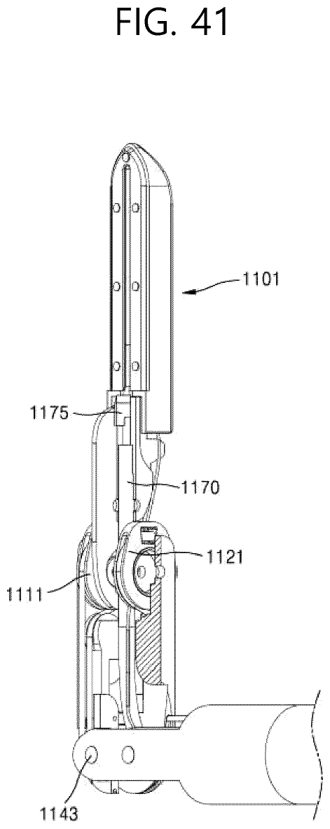

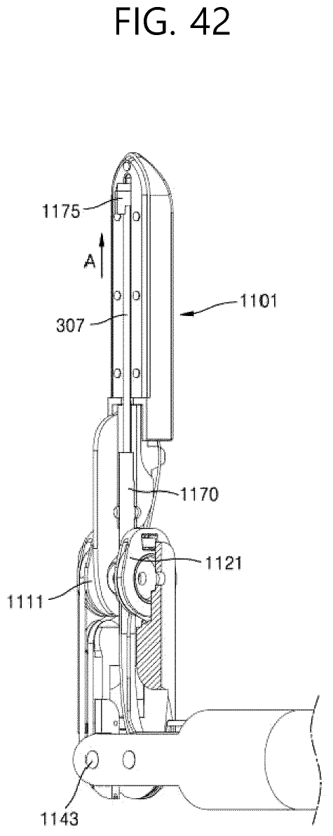

FIGS. 36 and 37 are views illustrating a process of performing an opening and closing motion in a state in which the end tool of the surgical instrument for electrocautery of FIG. 2 is pitch-rotated by +90°. FIGS. 38 and 39 are views illustrating a process of performing an opening and closing motion in a state in which the end tool of the surgical instrument for electrocautery of FIG. 2 is pitch-rotated by −90°. In addition, FIG. 40 is a view illustrating a path of the guide tube in a state in which the end tool of the surgical instrument for electrocautery of FIG. 2 is pitch-rotated by −90°. In addition, FIGS. 41 and 42 are views illustrating a path of the guide tube and a movement path of the blade during a cutting motion in a state in which the end tool of the surgical instrument for electrocautery of FIG. 2 is pitch-rotated by −90°.

As illustrated in FIGS. 36 to 42, the end tool of the surgical instrument for electrocautery according to an embodiment of the present disclosure is formed to be able to normally perform a cutting motion even in a state in which the jaws are pitch-rotated by −90°.

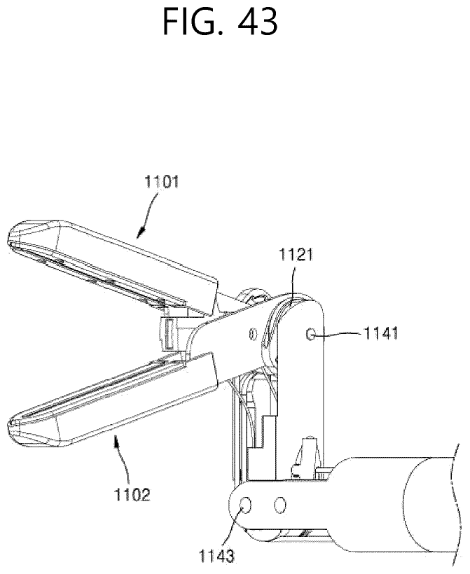

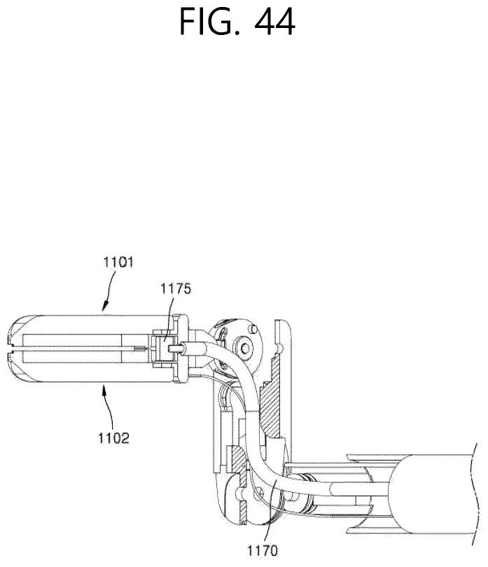

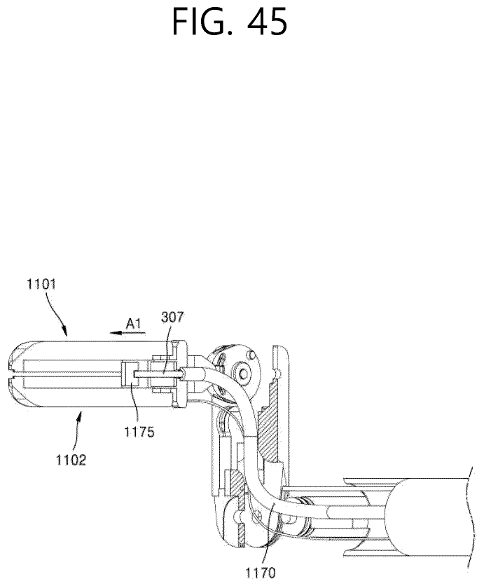

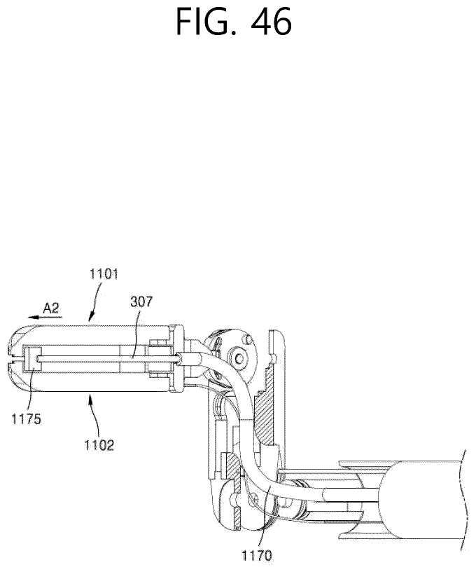

FIG. 43 is a perspective view illustrating a pitch-rotated state and a yaw-rotated state of the surgical instrument for electrocautery of FIG. 2, and FIGS. 44, 45, and 46 are perspective views illustrating a cutting motion of the end tool of the surgical instrument for electrocautery of FIG. 2 and illustrate a state of performing a cutting motion while the jaws are pitch-rotated by −90° and simultaneously yaw-rotated by +90°.

As illustrated in FIGS. 43 to 46, the end tool of the surgical instrument for electrocautery according to an embodiment of the present disclosure is formed to be able to normally perform a cutting motion even in a state in which the jaws are pitch-rotated by −90° and simultaneously yaw-rotated by +90°.

### Manipulation Unit

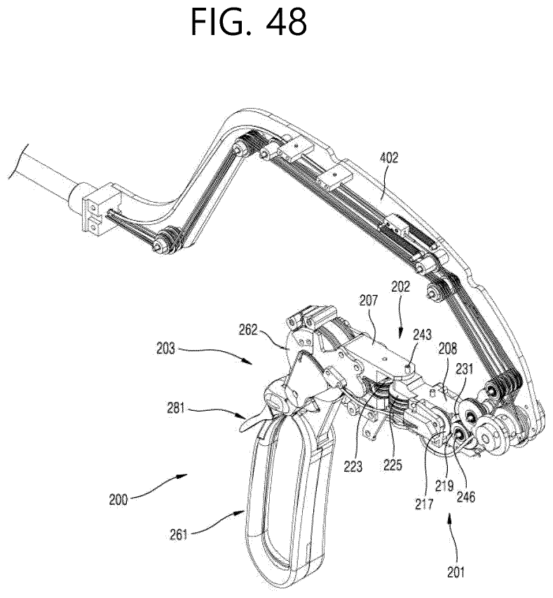

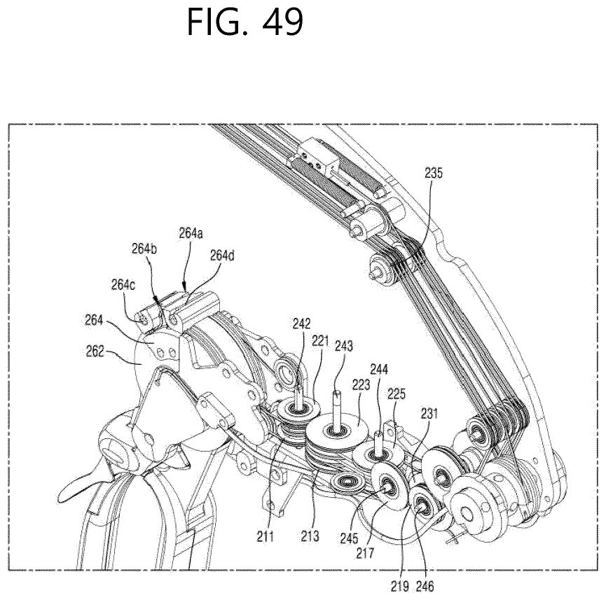

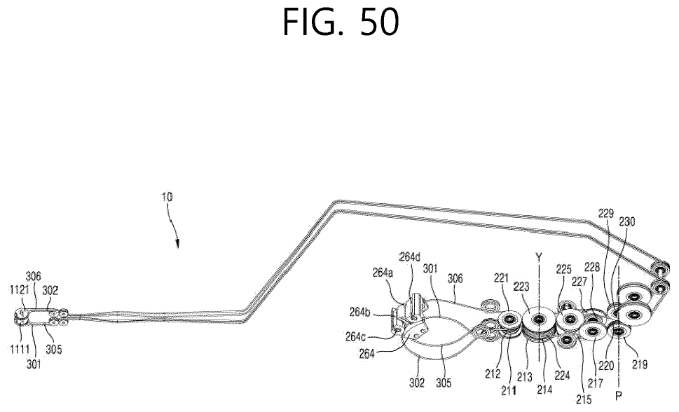

FIG. 47 is a perspective view illustrating the surgical instrument for electrocautery of FIG. 2. FIGS. 48 and 49 are perspective views illustrating a manipulation unit of the surgical instrument for electrocautery of FIG. 2. FIG. 50 is a view schematically illustrating only the configuration of pulleys and wires configuring joints of the surgical instrument for electrocautery of FIG. 2.

With reference to FIGS. 2 to 24 and 47 to 49, the manipulation unit 200 of the surgical instrument 10 for electrocautery according to an embodiment of the present disclosure includes a first handle 204 which a user may hold, the actuation manipulation unit 203 configured to control the actuation motion of the end tool 1100, the yaw manipulation unit 202 configured to control the yaw motion of the end tool 1100, and the pitch manipulation unit 201 configured to control the pitch motion of the end tool 1100. Herein, FIGS. 48 and 49 illustrate components only associated with the pitch/yaw/actuation motions of the surgical instrument 10 for electrocautery.

In addition, the manipulation unit 200 of the surgical instrument 10 for electrocautery may further include a cutting manipulation unit 280 performing cutting by controlling the movement of the blade 1175 of the end tool 1100, and a sealing manipulation unit 270 performing cautery by supplying electric energy to the first electrode 1151 and the second electrode 1152 of the end tool 1100.

The manipulation unit 200 may include a pulley 211, a pulley 212, a pulley 213, a pulley 214, a pulley 215, a pulley 217, and a pulley 218, a pulley 219, and a pulley 220, which are associated with the rotational motion of the first jaw 1101. In addition, the manipulation unit 200 may include a pulley 221, a pulley 222, a pulley 223, a pulley 224, a pulley 225, a pulley 227, a pulley 228, a pulley 229, and a pulley 230, which are associated with the rotational motion of the second jaw 1102. In addition, the manipulation unit 200 may include a pulley 262, which is associated with the rotational motion of the first jaw and the second jaw. In addition, the manipulation unit 200 may include a pulley 231, which is associated with a pitch motion. In addition, the manipulation unit 200 may include an intermediate pulley 235 disposed arranged in some positions of the bent unit 402 of the connection unit 400.

Herein, the drawings illustrate that the pulleys facing each other are arranged in parallel with each other; however, the concepts of the present disclosure are not limited thereto, and each pulley may be formed in various positions and sizes suitable for the configuration of the manipulation unit.

In addition, the manipulation unit 200 of an embodiment of the present disclosure may include a rotation axis 241, a rotation axis 242, a rotation axis 243, a rotation axis 244, a rotation axis 245, and a rotation axis 246. Herein, the rotation axis 241 may function as a manipulation portion first jaw actuation rotation axis, and the rotation axis 242 may function as a manipulation unit second jaw actuation rotation axis. In addition, the rotation axis 243 may function as a manipulation unit yaw main rotation axis, and the rotation axis 244 may function as a manipulation unit yaw subsidiary rotation axis. In addition, the rotation axis 245 may function as a manipulation unit pitch subsidiary rotation axis, and the rotation axis 246 may function as a manipulation unit pitch main rotation axis.

The rotation axis 241, the rotation axis 242, the rotation axis 243, the rotation axis 244, the rotation axis 245, and the rotation axis 246 may be sequentially disposed in a direction towards a proximal end 206 from a distal end 205.

One or more pulleys may be fitted into each of the rotation axes 241, 242, 243, 244, 245, and 246 which will be described in detail below.

The pulley 262 may function as a first jaw actuation pulley and a second jaw actuation pulley, and these components may be referred to as a manipulation unit actuation pulley.

The pulley 211 and the pulley 212 may function as a manipulation unit first jaw first yaw subsidiary pulley, the pulley 221 and the pulley 222 may function as a manipulation unit second jaw first yaw subsidiary pulley, and these two components may collectively be referred to as a manipulation unit yaw subsidiary pulley.

The pulley 213 and the pulley 214 may function as a manipulation unit first jaw yaw main pulley, the pulley 223 and the pulley 224 may function as a manipulation unit second jaw yaw main pulley, and these two components may collectively be referred to as a manipulation unit yaw main pulley.

The pulley 215 may function as a manipulation unit first jaw second yaw subsidiary pulley, the pulley 225 and the pulley 226 may function as a manipulation unit second jaw second yaw subsidiary pulley, and these two components may collectively be referred to as a manipulation unit second yaw subsidiary pulley.

The pulley 217 and the pulley 218 may function as a manipulation unit first jaw pitch subsidiary pulley, the pulley 227 and the pulley 228 may function as a manipulation unit second jaw pitch subsidiary pulley, and these two components may collectively be referred to as a manipulation unit pitch subsidiary pulley.

The pulley 219 and the pulley 220 may function as a manipulation unit first jaw pitch main pulley, the pulley 229 and the pulley 230 may function as a manipulation unit second jaw pitch main pulley, and these two components may collectively be referred to as a manipulation unit pitch main pulley.

The pulley 231 may function as a manipulation unit pitch wire main pulley, and may include a pulley (not shown) functioning as a manipulation unit pitch wire subsidiary pulley.

The components may be classified from the viewpoint of the manipulation unit in connection with each motion (pitch/yaw/actuation) as follows.

The pitch manipulation unit 201 controlling the pitch motion of the end tool 1100 may include a pulley 217, a pulley 218, a pulley 219, a pulley 220, a pulley 227, a pulley 228, a pulley 229, a pulley 230, and a pulley 231. In addition, the pitch manipulation unit 201 may include the rotation axis 245 and the rotation axis 246. In addition, the pitch manipulation unit 201 may further include a pitch frame 208.

The yaw manipulation unit 202 controlling the yaw motion of the end tool 1100 may include a pulley 211, a pulley 212, a pulley 213, a pulley 214, a pulley 215, a pulley 221, a pulley 222, a pulley 223, a pulley 224, and a pulley 225. In addition, the yaw manipulation unit 202 may include the rotation axis 242, the rotation axis 243, and the rotation axis 244. In addition, the yaw manipulation unit 202 may further include a yaw frame 207.

The actuation manipulation unit 203 controlling the actuation motion of the end tool 1100 may include a pulley 262 and a rotation axis 241.

Hereinafter, each component of the manipulation unit 200 will be described in more detail.

The first handle 204 may be held by a user, and more particularly, a user may hold the first handle 204 by wrapping the same with his or her hand. The actuation manipulation unit 203 and the yaw manipulation unit 202 may be formed on the first handle 204, and the pitch manipulation unit 201 may be formed on one side of the yaw manipulation unit 202. In addition, another end of the pitch manipulation unit 201 may be connected to the bent portion 402 of the connection unit 400.

The actuation manipulation unit 203 may include an actuation lever 261, an actuation pulley 262, and an actuation restoration elastic member 263.

Herein, the actuation lever 261 is formed in the shape of a hand ring and may operate as a second handle.

Herein, the rotation axis 241, which is an actuation rotation axis, may be formed to form a predetermined angle with an XZ plane on which the connection unit 400 is formed.

For example, the rotation axis 241 may be formed in a direction parallel to the Z-axis, and in this state, when the pitch manipulation unit 201 or the yaw manipulation unit 202 is rotated, the coordinate system of the actuation manipulation unit 203 may change relatively. The concept of the present disclosure is not limited thereto, and the rotation axis 241 may be formed in various directions so as to be suitable for a structure of the hand of the user gripping the actuation manipulation unit 203 according to an ergonomic design.

The actuation pulley 262 may be fixedly coupled to the actuation lever 261, or may be formed as a single member. Accordingly, the actuation pulley 262 may rotate together with the rotation of the actuation lever 261.

Herein, the actuation pulley 262 may be configured to be a single pulley or two pulleys fixedly coupled to each other.

The yaw manipulation unit 202 may include the rotation axis 242, the rotation axis 243, the pulley 213 and the pulley 214, which are the manipulation unit first jaw yaw main pulley, the pulley 223 and the pulley 224, which are the manipulation unit second jaw yaw main pulley, and the yaw frame 207. In addition, the yaw manipulation unit 202 may further include the pulley 211 and the pulley 212, which are the manipulation unit first jaw first yaw subsidiary pulley and arranged on one side of the pulley 213 and the pulley 214, and the pulley 221 and the pulley 222, which are the manipulation unit second jaw first yaw subsidiary pulley and arranged on one side of the pulley 223 and the pulley 224. In addition, the yaw manipulation unit 202 may further include the pulley 215, which is the manipulation unit first jaw second yaw subsidiary pully arranged on another side of the pully 213 and the pulley 214, and the pulley 225, which is the manipulation unit second jaw second yaw subsidiary pulley arranged on another side of the pulley 223 and the pulley 224. Herein, the pulley 215 and the pulley 225 may be coupled to the pitch frame 208 to be described later.

Herein, the drawings illustrate that the yaw manipulation unit 202 includes the pulley 213, the pulley 214, the pulley 223, and the pulley 224, and as the pulley 213 and the pulley 214 face the pulley 224, two pulleys may be rotatable independently of each other; however, the concepts of the present disclosure are not limited thereto. In other words, one or more pulleys having the same diameter or different diameters may be provided according to the configuration of the yaw manipulation unit 202.

Specifically, on the first handle 204, the rotation axis 242, which is the manipulation unit first yaw subsidiary rotation axis, is formed on one side of the actuation manipulation unit 203, and the rotation axis 243, which is the manipulation unit yaw main rotation axis, is formed on one side of the rotation axis 242. In this connection, the first handle 204 may be formed to be rotatable around the rotation axis 243.

Herein, the rotation axis 243 may be formed to have a certain angle with the XY plane on which the connection unit 400 is formed. For example, the rotation axis 243 may be formed in a direction parallel with the Z-axis, and when the pitch manipulation unit 201 rotates, the coordinate system of the rotation axis 243 may be changed relatively as described above. However, the concepts of the present disclosure are not limited thereto, and by an ergonomic design, the rotation axis 243 may be formed in various directions suitable for a hand structure of a user holding the manipulation unit 200.

The pulley 213, the pulley 214, the pulley 223, and the pulley 224 may be coupled to the rotation axis 243 to be rotatable around the rotation axis 243. In addition, the wire 301 or the wire 305, which is the first jaw wire, may be wound around the pulley 213 and the pulley 214, and the wire 302 or the wire 306, which is the second jaw wire, may be wound around the pulley 223 and the pulley 224. In this connection, as the pulley 213 faces the pulley 214, and the pulley 223 faces the pulley 224, there may be two pulleys which are rotatable independently. Accordingly, as the wire wound inward and the wire wound outward may be respectively wound around separate pulleys, the pulleys may operate without interfering with each other.

The yaw frame 207 may rigidly connect the first handle 204, the rotation axis 242, and the rotation axis 243; and the actuation manipulation unit 203, in which the rotation axis 241 and the actuation pulley 262 are coupled, is rigidly connected to the yaw frame 207 directly or through an intermediate member. Accordingly, the first handle 204, the yaw manipulation unit 202, and the actuation manipulation unit 203 may yaw-rotate around the rotation axis 243 in an integrated manner.

The pitch manipulation unit 201 may include the rotation axis 246, the pulley 217 and the pulley 218, which are the manipulation unit first jaw pitch main pulley, the pulley 229 and the pulley 230, which are the manipulation unit second jaw pitch main pulley, and the pitch frame 208. In addition, the pitch manipulation unit 201 may further include the rotation axis 245, the pulley 217 and the pulley 218, which are the manipulation unit first jaw pitch subsidiary pulley and arranged on one side of the pulley 219 and the pulley 220, and the pulley 227 and the pulley 228, which are the manipulation unit second jaw pitch subsidiary pulley and arranged on one side of the pulley 229 and pulley 230. The pitch manipulation unit 201 may be connected to the bent unit 402 of the connection unit 400 through the rotation axis 246.

Specifically, the pitch frame 208 may be a base frame of the pitch manipulation unit 201, and one end of the pitch frame 208 may be rotatably coupled to the rotation axis 243. In other words, the yaw frame 207 may be formed to be rotatable around the rotation axis 243 with respect to the pitch frame 208.

As described above, the yaw frame 207 may connect the first handle 204, the rotation axis 243, the rotation axis 241, and the rotation axis 242, and as the yaw frame 207 is axially coupled to the pitch frame 208, when the pitch frame 208 pitch-rotates around the rotation axis 246, the yaw frame 207, the first handle 204, the rotation axis 241, the rotation axis 242, and the rotation axis 243, which are connected to the pitch frame 208, may also pitch rotate. In other words, when the pitch manipulation unit 201 rotates around the rotation axis 246, the actuation manipulation unit 203 and the yaw manipulation unit 202 may be rotated together with the pitch manipulation unit 201. In other words, when a user pitch-rotates the first handle 204 around the rotation axis 246, the actuation manipulation unit 203, the yaw manipulation unit 202, and the pitch manipulation unit 201 may also move together with the first handle 204.

The pulley 219, the pulley 220, the pulley 229, and the pulley 230 may be coupled to the rotation axis 246 so as to be rotatable around the rotation axis 246 of the pitch frame 208.

Herein, the pulley 219 and the pulley 220 may face each other and rotate independently. Accordingly, as the wire wound inward and the wire wound outward may be respectively wound around separate pulleys, the pulleys may operate without interfering with each other. As such, the pulley 229 and the pulley 230 may face each other and rotate independently. Accordingly, as the wire wound inward and the wire wound outward may be respectively wound around separate pulleys, the pulleys may operate without interfering with each other.

Next, the motions of the wire 303 and the wire 304 which are the pitch wire are described below.

In the end tool 1100, the pulley 1131, which is the end tool pitch pulley, may be fixedly coupled to the end tool hub 1180, and in the manipulation unit 200, the pulley 231 and the pulley 232 (not shown), which are the manipulation unit pitch pulley, may be fixedly coupled to the pitch frame 208. In addition, these pulleys may be connected to each other by the wire 303 and the wire 304, which are the pitch wire, to facilitate the pitch motion of the end tool 1100 according to the pitch manipulation of the manipulation unit 200. Herein, the wire 303 may be fixedly coupled to the pitch frame 208 via the pulley 231 and the wire 304 may be fixedly coupled to the pitch frame 208 via the pulley 232 (not shown). In other words, the pitch frame 208, the pulley 231, and the pulley 232 may rotate together around the rotation axis 246 by the pitch rotation of the manipulation unit 200. As a result, the wire 303 and the wire 304 may also move, and separately from the pitch motion of the end tool by the wire 301, the wire 302, the wire 305, and the wire 306, which are the jaw wire, additional pitch rotation power may be transmitted.

The connection relation among the first handle 204, the pitch manipulation unit 201, the yaw manipulation unit 202, and the actuation manipulation unit 203 is described below. On the first handle 204, the rotation axis 241, the rotation axis 242, the rotation axis 243, the rotation axis 244, the rotation axis 245, and the rotation axis 246 may be formed. In this connection, as the rotation axis 242 and the rotation axis 243 are directly formed on the first handle 204, the first handle 204 and the yaw manipulation unit 202 may be directly connected to each other. As the pitch manipulation unit 201 is arranged on one side of the yaw manipulation unit 202 and connected to the yaw manipulation unit 202, the pitch manipulation unit 201 may not be directly connected to the first handle 204 and the pitch manipulation unit 201 and the first handle 204 may be indirectly connected to each other through the yaw manipulation unit 202. In addition, since the actuation manipulation unit 203 is formed on the other side of the yaw manipulation unit 202 to be connected to the yaw manipulation unit 202, the actuation manipulation unit 203 may not be directly connected to the first handle 204, and the actuation manipulation unit 203 and the first handle 204 may be formed to be indirectly connected through the yaw manipulation unit 202.

With reference to the drawings, in the surgical instrument 10 for electrocautery according to an embodiment of the present disclosure, the pitch manipulation unit 201 and the end tool 1100 may be formed on the same or parallel axis (the X-axis). In other words, the rotation axis 246 of the pitch manipulation unit 201 may be formed at one end of the bent unit 402 of the connection unit 400, and the end tool 1100 may be formed at the other end of the connection unit 400.

In addition, one or more intermediate pulleys 235 changing or guiding a path of the wires may be disposed in some positions of the connection unit 400, in particular, in positions on the bent unit 402. At least a portion of the wires may be wound around the intermediate pulleys 235 to guide the path of the wires so that the wires are disposed along the bent shape of the bent unit 402.

Herein, the drawings illustrate that the connection unit 400 includes the bent unit 402, and thus is formed in a curved manner with a certain curvature; however, the concepts of the present disclosure are not limited thereto, and the connection unit 400 may be formed straightly, if necessary, or curved in one or more points. Even in such cases, the pitch manipulation unit 201 and the end tool 1100 may be formed on the substantially same or parallel axis. In addition, although FIG. 2 illustrates that the pitch manipulation unit 201 and the end tool 1100 are respectively formed on an axis parallel with the X-axis, the concepts of the present disclosure are not limited thereto, and the pitch manipulation unit 201 and the end tool 1100 may be formed on different axes.

### Actuation Motion, Yaw Motion, and Pitch Motion

Actuation motion, yaw motion, and pitch motion in this embodiment will be described as follows.

First, the actuation motion is as follows.

When a user puts a finger in a hand ring formed at the actuation lever 261 and rotates the actuation lever 261 using the finger(s), the actuation pulley 262 fixedly coupled to the actuation lever 261 rotates around the rotation axis 241.

In this connection, the wire 301 and wire 305 wound with one end fixedly coupled to the pulley 262, and the wire 302 and wire 306 wound with one end fixedly coupled to the same pulley 262 move as the pulley 262 rotates. Herein, the wires 301, 302, 305, and 306 are coupled to one actuation pulley 262, but the movement of the wire as the pulley rotates varies depending on the direction in which each wire is wound around the pulley 262. This will be explained in detail later.

In addition, this rotational force is transmitted to an end tool 1100 through the power transmission unit 300, two jaws 1103 of the end tool 1100 perform the actuation motion.

Herein, the actuation motion refers to a motion of opening or closing the jaws 1101 and 1102 while the two jaws 1101 and 1102 rotate in opposite directions to each other, as described above. In other words, when the actuation lever 261 of the actuation manipulation unit 203 is rotated in a direction closer to the first handle 204, the first jaw 1101 rotates counterclockwise and the second jaw 1102 rotates clockwise, and thus the end tool 1100 is closed. Conversely, when the actuation lever 261 of the actuation manipulation unit 203 is rotated in a direction away from the first handle 204, the first jaw 1121 rotates clockwise and the second jaw 1122 rotates counterclockwise, and thus the end tool 1100 is opened.

Next, the yaw motion is as follows.

When a user rotates the first handle 204 around the rotation axis 243 while holding the first handle 204, the actuation manipulation unit 203 and the yaw manipulation unit 202 yaw-rotates around the rotation axis 243. In other words, when the actuation pulley 262 to which the wire 301 and the wire 305 are fixedly coupled rotates about the rotation axis 243, the wire 301 and the wire 305 wound around the pulley 213 and the pulley 214 move. In this connection, one of the wires 301 and 305 is wound around the pulley 213 or the pulley 214, and the other one of the wire 301 and the wire 305 is unwound from the pulley 213 or pulley 214. As such, since the wire 302 and the wire 306 are also fixedly coupled to the actuation pulley 262, when the actuation pulley 262 rotates around the rotation axis 243, the wire 302 and the wire 306 wound around the pulley 223 and the pulley 224 move. In this connection, one of the wires 302 and 306 is wound around the pulley 223 or the pulley 224, and the other one of the wire 302 and the wire 306 is unwound from the pulley 223 or pulley 224. In this connection, the wire 301 and the wire 305 connected to the first jaw 1101 and the wire 302 and the wire 306 connected to the second jaw 1102 are wound around the pulley 213 and the pulley 214 and the pulley 223 and the pulley 224, such that the first jaw 1101 and the second jaw 1102 rotate in the same direction during a yaw rotation. In addition, this rotational force is transmitted to the end tool 1100 through the power transmission unit 300, and the two jaws 1103 of the end tool 1100 performs the yaw motion that rotates in the same direction.

In this connection, since the yaw frame 207 connects the first handle 204, the rotation axis 241, the rotation axis 242, and the rotation axis 243, the first handle 204, the yaw manipulation unit 202, and the actuation manipulation unit 203 rotate together around the rotation axis 243.

Next, the pitch motion is as follows.

When a user rotates the first handle 204 around the rotation axis 246 while holding the first handle 204, the actuation manipulation unit 203, the yaw manipulation unit 202, and the pitch manipulation unit 201 make pitch rotation around the rotation axis 243. In other words, when the actuation pulley 262 to which the wire 301 and the wire 305 are fixedly coupled rotates about the rotation axis 246, the wire 301 and the wire 305 wound around the pulley 219 and the pulley 220 move. As such, when the actuation pulley 262 to which the wire 302 and the wire 306 are fixedly coupled rotates about the rotation axis 246, the wire 302 and the wire 306 wound around the pulley 229 and the pulley 230 move. In this connection, as described above with reference to FIG. 50, the wire 301, the wire 305, the wire 302, and the wire 306, which are jaw wires, are wound around the pulley 219, the pulley 220, the pulley 229, and the pulley 230, which are manipulation unit pitch main pulleys, such that the wire 301 and wire 305, which are first jaw wires, move in the same direction, and the wire 302 and the wire 306, which are second jaw wires, move in the same direction to enable pitch rotation of the first jaw 1101 and the second jaw 1102. In addition, this rotational force is transmitted to the end tool 1100 through the power transmission unit 300, and two jaws 1103 of the end tool 1100 perform the pitch motion.

In this connection, the pitch frame 208 is connected to the yaw frame 207 and the yaw frame 207 connects the first handle 204, the rotation axis 241, the rotation axis 242, and the rotation axis 243. Accordingly, when the pitch frame 208 rotates around the rotation axis 246, the yaw frame 207 connected to the pitch frame 208, the first handle 204, the rotation axis 241, the rotation axis 242, and the rotation axis 243 rotate together. In other words, when the pitch manipulation unit 201 rotates around the rotation axis 246, the actuation manipulation unit 203 and the yaw manipulation unit 202 are rotated together with the pitch manipulation unit 201.

To sum up, the surgical instrument 10 for electrocautery according to an embodiment of the present disclosure is configured such that pulleys are formed at each joint point (actuation joint, yaw joint, and pitch joint), wire (first jaw wire or second jaw wire) is wound on the pulley, and rotational manipulation of the manipulation unit (actuation rotation, yaw rotation, and pitch rotation) causes movement of each wire. As a result, a desired motion of the end tool 1100 is induced. Furthermore, auxiliary pulleys may be formed on one side of each pulley, and the wire may not be wound several times on one pulley by these auxiliary pulleys.

FIG. 50 is a view schematically illustrating only the configuration of pulleys and wires configuring joints of the surgical instrument 10 for electrocautery according to an embodiment of the present disclosure illustrated in FIG. 2. In FIG. 50, intermediate pulleys that are for changing paths of wires and are not associated with joint motions are omitted.

Referring to FIG. 50, the manipulation unit 200 may include the pulley 211, the pulley 212, the pulley 213, the pulley 214, the pulley 215, the pulley 217, the pulley 218, the pulley 219, and the pulley 220 that are associated with the rotational motion of the first jaw 1101.

In addition, the manipulation unit 200 may include the pulley 221, the pulley 222, the pulley 223, the pulley 224, the pulley 225, the pulley 227, the pulley 228, the pulley 229, and the pulley 230 associated with the rotational motion of the second jaw 1102. In addition, the manipulation unit 200 may include the pulley 262 associated with the rotational motions of the first jaw and the second jaw. (The disposition and the configuration of pulleys in the manipulation unit 200 are the same as the disposition and the configuration of the pulleys in the end tool 1100 in principle, and thus some of the reference numerals thereof will be omitted in the drawings.)

The pulley 211 and the pulley 212 and the pulley 221 and the pulley 222 may be formed to be rotatable independently of each other around the same axis, that is, the rotation axis 243. In this connection, the pulley 211 and the pulley 212 may be formed to face each other, thereby forming two independently rotatable pulleys. As such, the pulley 221 and the pulley 222 may be formed of two pulleys that are formed to face each other and be rotatable independently. In this connection, the two pulleys may be formed to have different diameters.

The pulley 213 and the pulley 214 and the pulley 223 and the pulley 224 may be formed to be rotatable independently of each other around the same axis, that is, the rotation axis 243. In this connection, the pulley 213 and the pulley 214, respectively, and the pulley 223 and 224 may be formed of two pulleys that face each other and may rotate independently.

The pulley 215 and the pulley 225 may be formed to be rotatable independently of each other around the same axis, that is, the rotation axis 244.

The pulley 217 and the pulley 218 and the pulley 227 and the pulley 228 may be formed to be rotatable independently of each other around the same axis, that is, the rotation axis 245. In this connection, the pulley 217 and pulley 218 may be formed to have different diameters. Additionally, the pulley 227 and pulley 228 may be formed to have different diameters.

The pulley 219 and pulley 220 and the pulley 229 and pulley 230 may be formed to rotate independently of each other around the same axis, the rotation axis 246.

The wire 301 sequentially passes through the pulley 219, the pulley 217, the pulley 215, the pulley 213, and the pulley 211 of the manipulation unit 200, is wound around the pulley 262, and then is coupled to the pulley 262 by the fastening member 324. The wire 305 sequentially passes through the pulley 220, the pulley 218, the pulley 214, the pulley 212 of the manipulation unit 200 and is coupled to the pulley 262 by the fastening member 264c. Herein, the fastening members 264a and 264c may be directly coupled to the pulley 262, or the fastening members 264a and 264c may be coupled to a separate coupling member 264 and the coupling member 254 may be coupled to the pulley 262, so that the fastening members 264a and 264c may be coupled to the pulley 262. Accordingly, as the pulley 262 rotates, the wire 301 and the wire 305 are wound around or unwound from the pulley 262, and thus the first jaw 1101 rotates.

The wire 306 sequentially passes through the pulley 229, the pulley 227, the pulley 225, the pulley 223, and the pulley 221 of the manipulation unit 200, is wound around the pulley 262, and then is coupled to the pulley 262 by a fastening member 264d. The wire 302 sequentially passes through the pulley 230, the pulley 228, the pulley 224, and the pulley 222 of the manipulation unit 200 and is coupled to the pulley 262 by the fastening member 264b. Herein, the fastening members 264b and 264d may be directly coupled to the pulley 262, or the fastening members 264b and 264d may be coupled to a separate coupling member 264 and the coupling member 254 may be coupled to the pulley 262, so that the fastening members 264b and 264d may be coupled to the pulley 262. Accordingly, as the pulley 262 rotates, the wire 302 and the wire 306 are wound around or unwound from the pulley 262, and thus the second jaw 1102 rotates.

### Electric Energy Control

As described above, recently, it has become possible to use an electric surgical instrument that uses electric energy to perform certain surgical tasks. For example, in surgical instruments such as graspers, scissors, tweezers, blades, needles, and hooks, electric surgical instruments including one or more electrodes formed to receive electric energy have been developed. Electric energy supplied through the electrodes may be used to coagulate, bond, or cut the patient's body tissues. In particular, when electric energy is used, amputation and hemostasis may be performed at the same time.

Electric surgical instruments are typically divided into two types: monopolar and bipolar. In a monopolar electric surgical instrument, electric energy of a specific polarity is supplied to one or more electrodes of the instrument. In addition, electricity of different polarity is electrically connected to the patient. In a bipolar electric surgical instrument, one or more electrodes are electrically connected to a first polarity electric energy source, and one or more electrodes are electrically connected to a second polarity electric energy source opposite to the first polarity.

Hereinafter, the configuration of an exemplary surgical device for electrocautery is described for convenience of explanation. For example, the surgical device for electrocautery according to an aspect may include a bipolar electrosurgical instrument having a first jaw and a second jaw, and each jaw having an electrode. However, the technical idea of the present disclosure is not limited thereto, and it should be understood that each of the technical features of the embodiments described in this description may be applied to monopolar and bipolar electrosurgical instruments as well as devices for performing surgery based on arbitrary electric energy having various structures.

In this regard, thus far, various types of surgical instruments for electrocautery have been developed. Among the various types of surgical instruments for electrocautery, a blood vessel sealing device called “Vessel Sealer” has a sensing function added to the existing bipolar cautery method, so that power of different polarities may be supplied to two electrodes, and after denaturing a vessel with the heat generated therefrom for hemostasis, the stanched part may be cut with a blade. In this connection, the impedance of the tissue (or blood vessel) while the current is flowing is measured to determine whether the cauterization is completed, and when the cauterization is completed, the current supply is automatically stopped, and the tissue is cut with the blade.

An exemplary vessel sealing device may include a body (for example, an electrosurgical machine (RF generator)) and an end tool (for example, an electrosurgical tool). After connecting the cable of the electrosurgical tool to the electrosurgical machine, the button on the handpiece may be activated to output electric energy to the end tool. Heat is applied to the tissues or blood vessels in contact with the end tool by the electric energy. For example, blood vessels may be sealed within seconds.

As previously explained with reference to FIGS. 2 to 24, the end tool 1100 of an embodiment of the present disclosure may include the first jaw 1101, the second jaw 1102, the first electrode 1151, the second electrode 1152, the guide tube 1170, and the blade 1175 in order to perform cauterizing and cutting motions.

Herein, the first electrode 1151 may be formed on a surface of the first jaw 1101 facing the second jaw 1102. In addition, the second electrode 1152 may be formed on a surface of the second jaw 1102 facing the first jaw 1101.

In this connection, the slit 1151a may be formed in the first electrode 1151, and the blade 1175 may move along the slit 1151a. In addition, the slit 1152a may be formed in the second electrode 1152, and the blade 1175 may move along the slit 1152a.

Although not illustrated in the drawings, one or more sensors (not shown) may be further formed on at least one of the first jaw 1101 or the second jaw 1102. The sensor (not shown) may be formed to measure at least some of current, voltage, resistance, impedance, and temperature during the cautery by locating tissue between the first jaw 1101 and the second jaw 1102 and passing a current through the first electrode 1151 and the second electrode 1152.

Alternatively, instead of providing a separate sensor, monitoring and control of at least some of a current, a voltage, a resistance, an impedance, and a temperature may be directly performed by a generator (not shown) which supplies power to the electrodes.

As such, the end tool for electrocautery surgery according to an aspect is provided with a sensor for sensing at least some of current, voltage, resistance, impedance, and temperature, or the generator itself monitors and controls the same. This is because in a surgical device for electrocautery, it is very important to control the output level of electric energy to an appropriate level in order to minimize tissue damage and achieve rapid sealing of blood vessels. The amount, intensity, or time of electric energy applied to the target tissue to perform the cauterization surgery may be determined differently depending on the thickness of target tissue or degree of cauterization. For example, when excessive electric energy is supplied to thin tissue compared to that required, significant damage may occur to the tissue. On the other hand, when less than the standard amount of electric energy is supplied to thick tissue, the time required for electrocautery may be very long, or even cauterization of the target tissue may not be achieved as needed.

In order to appropriately control supply electric energy, a procedure has been used to measure the impedance of the target tissue while cauterization is performed by flowing a current, as described above, and to control the supply electric energy based thereon. In other words, it is possible to determine the impedance of the target tissue based on the voltage and/or current supplied to the end tool by a sensor or generator, and determine the end point of supply of electric energy by utilizing a specific value of impedance or automatically determining the same through a time series pattern of impedance. However, such impedance-based control of the supply electric energy may have the limitation of not being able to distinguish impedance changes due to changes in tissue thickness and differences in tissue components such as moisture and fat content within the tissue.

For example, a tissue with a lot of moisture may have a lower impedance value measured than a tissue with less moisture, and similarly, even when the thickness of the tissue is very thin, the impedance value may be measured as a low value. In this connection, there is an issue in that whether the low measured impedance value is due to a lot of moisture in the tissue or a thin tissue thickness cannot be determined based on the impedance measurement value alone. For example, in the case where the target tissue is actually a very thick tissue but contains a lot of moisture, when the thickness of the tissue is determined utilizing only the impedance measurement value, it may be mistakenly determined that the target tissue is a very thin tissue. Furthermore, there is an issue that the supply time of electric energy may be shortened or the electric energy may be supplied weakly. Accordingly, there may be cases where the electrocautery procedure for the tissue takes a very long time or the tissue is not properly cauterized. In other words, impedance measurement alone cannot take into account the characteristics of the target tissue, so there is a limitation in that appropriate control of the supply electric energy may not be performed.

The surgical device for electrocautery according to an embodiment of the present disclosure is intended to address the associated issue, and may determine component information of the target tissue through light irradiation and response light analysis in a predetermined band to the target tissue, and may reflect the same to control the electric energy supplied to the target tissue. Accordingly, it is possible to perform a quick electrocautery surgical procedure while minimizing tissue damage to the target tissue. As a non-limiting example, the surgical device for electrocautery according to an aspect of the present disclosure includes an electrosurgical machine and an electrosurgical instrument, wherein the electrosurgical machine includes a spectrometer and the electrosurgical instrument includes an optical unit to transmit light to tissue and measure the content of constituents in the tissue. Accordingly, it is possible to provide a blood vessel sealing system including an optical-based feedback system. Thus, the limitations of the current-based impedance feedback system may be resolved. By distinguishing the state of the tissue, the size and supply time of electric energy may be precisely determined.

Hereinafter, with reference to the drawings, a surgical device for electrocautery according to an embodiment of the present disclosure will be described in more detail.

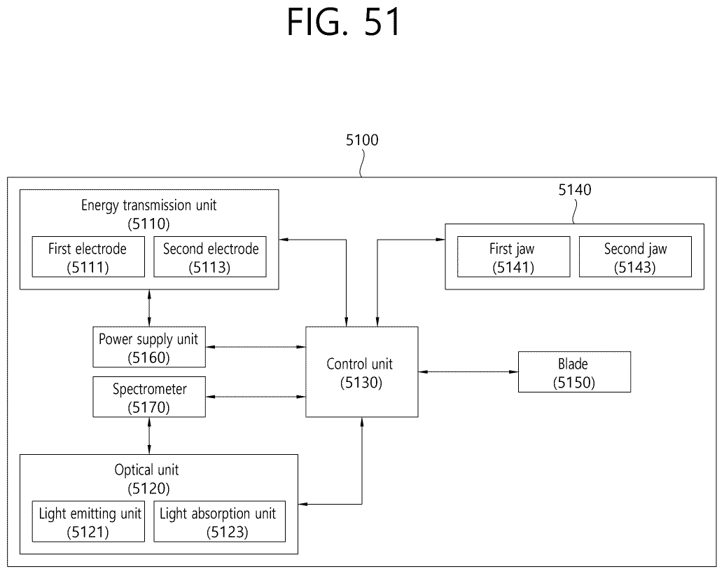

FIG. 51 is a block diagram illustrating an exemplary configuration of a surgical device for electrocautery according to an embodiment of the present disclosure.

As illustrated in FIG. 51, a surgical device 5100 for electrocautery according to an embodiment of the present disclosure may be configured to grip a target tissue based on a pair of jaws 5140 including a first jaw 5141 and a second jaw 5143, for example, to seal the same based on electric energy, for example, when the target tissue is a blood vessel, and then to cut the sealed blood vessel using a blade 5150. However, this is merely an example and the technical idea of the present disclosure is not limited thereto. It should be understood that the technical features according to the embodiments of the present disclosure may be applied to any device that performs electrocautery surgery based on electric energy.

As illustrated in FIG. 51, the surgical device 5100 for electrocautery according to an embodiment of the present disclosure may adjust the electric energy supplied from a power supply unit 5160 to an appropriate level and transmits the same to the target tissue through an energy transmission unit 5110. As a non-limiting example, the energy transmission unit 5110 may include, for example, a first electrode 5111 and a second electrode 5113. As described with reference to FIGS. 2-24 previously in this description, the first electrode may be formed on the surface facing the second jaw in the first jaw, and the second electrode may be formed on the surface facing the first jaw in the second jaw, but are not limited thereto.

A control unit 5130 may control the electric energy supplied to the target tissue through the energy transmission unit 5110 based on optical information on the target tissue obtained using an optical unit 5120. For example, the control unit 5130 may control at least a portion of the supply time and/or intensity of the electric energy supplied to the target tissue, and may be configured to determine when to stop supplying the electric energy to the target tissue.

As a non-limiting example, the control unit 5130 may acquire optical information on the target tissue using a spectrometer 5170 and/or the optical unit 5120, and use such optical information to determine tissue component information on the components of the target tissue. For example, information on moisture or fat content may be determined and used together with the impedance measurement results to control the electric energy supplied to the target tissue. Control of the supply electric energy of the control unit 5130 is explained in more detail later in this description.

The spectrometer 5170 may include any constituents capable of generating light in a predetermined wavelength range and performing analysis on light in a specific wavelength band. For example, the control unit 5130 may use the spectrometer 5170 to receive light in a wavelength band corresponding to the component for determining the information contained in the target tissue, and may irradiate the target tissue with light in a wavelength band corresponding to the component to be determined, collect the response light from the target tissue, and analyze the same. According to an aspect, the optical unit 5120 may be provided on the end tool for irradiating and collecting light. As a non-limiting example, the optical unit 5120 may be disposed in a direction in contact with the target tissue of the first jaw. The optical unit 5120 may include a light emitting unit 5121 for irradiating light in a wavelength band corresponding to the component to be determined to the target tissue, and a light absorption unit 5123 for collecting response light from the target tissue. Using the optical unit 5120, the control unit 5130 may be configured to irradiate light of a predetermined wavelength band to the target tissue, collect response light from the target tissue and determine tissue component information based thereon, and control the supply electric energy to the target tissue through the energy transmission unit.

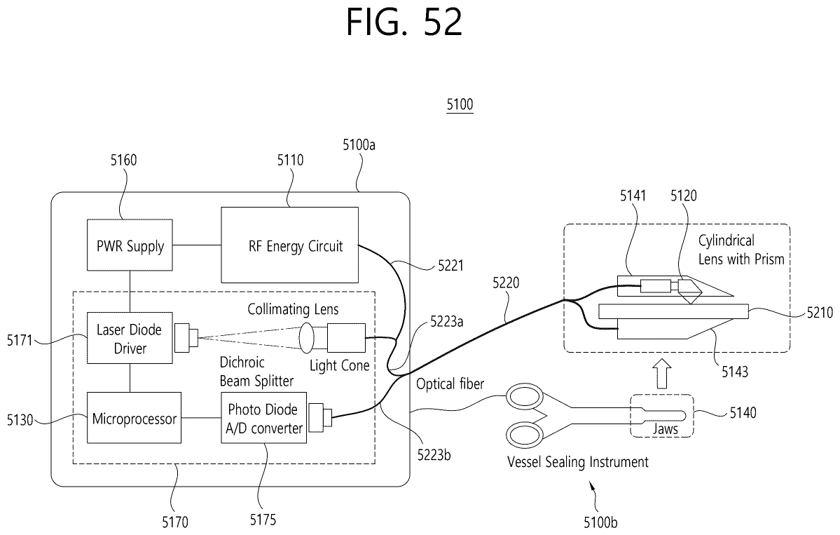

FIG. 52 is an exemplary implementation example of the surgical device for electrocautery of FIG. 51. For example, as illustrated in FIG. 52, the surgical device 5100 for electrocautery may include a body unit 5100a and an end tool 5100b. However, the configuration in FIG. 51 is merely an example, and unlike the configuration illustrated in FIG. 51, the surgical device 5100 for electrocautery may be configured as an integrated piece, or, unlike the configuration of the exemplary body unit and the end tool in FIG. 51, some constituents may be disposed elsewhere in the body unit 5100a and/or the end tool 5100b.

In the implementation example of FIG. 52, exemplary implementation configurations for implementing the configuration of FIG. 51 are indicated through the same reference numerals. As illustrated in FIG. 52, the end tool 5100b may be, for example, a Vessel Sealing Instrument. In this description, the end tool 5100b may be referred to as a ‘handpiece,’ but the end tool 5100b of this description is not limited to an instrument that is directly held by a user. For example, in a robotic surgery system, an end tool held by a robot arm should also be understood to be included in the technical idea of the present disclosure.

Referring to FIG. 52, the end tool 5100b is provided with the pair of jaws 5140 including the first jaw 5141 and the second jaw 5143 and is configured to grip a target tissue or blood vessel 5210. In other words, the end tool may include the first jaw 5141 and the second jaw 5143 for gripping the target tissue. In an aspect, the energy transmission unit may include an RF energy circuit 5110, the first electrode 5111 provided in the first jaw, and the second electrode 5113 provided in the second jaw.

The end tool 5100b may also be provided with an optical module 5120, for example a cylindrical lens with a prism. According to an aspect, the optical unit may be configured to be disposed in either the first jaw 5141 or the second jaw 5143 to irradiate light in a predetermined wavelength band to the target tissue and collect reflected light from the target tissue. In addition, the end tool 5100b may be further provided with a blade 5150 that cuts the target tissue while moving between the proximal end and the distal end of either the first jaw or the second jaw. According to an aspect, the optical unit 5120 may be disposed in the first jaw 5141, and the blade 5150 may be disposed in the second jaw 5143, without being limited thereto. According to another embodiment of the present disclosure, when the light irradiated by the light emitting unit 5121 passes through the target tissue, the light absorption unit 5123 disposed opposite the light emitting unit 5121 may be configured to collect light passing through the target tissue as response light for the target tissue. In this connection, for example, the light emitting unit 5121 may be disposed in the first tank 5141, and the light absorption unit 5123 may be disposed in the second jaw 5143. However, according to an aspect of the present disclosure, considering that the blood vessel sealing device is required to be provided with a blade for cutting the blood vessel after sealing, the first jaw may be provided with both the light emitting unit 5121 and the light absorption unit 5123, the light absorption unit 5123 may be configured to collect reflected light from the target tissue, and the blade may be configured to be disposed in the second jaw.

As a non-limiting example, the first jaw 5141 in which the optical module 5120 is disposed may be an upper jaw. In other words, the optical module 5120 may be disposed in the upper jaw of the end tool 5100b. In a typical surgical procedure, among the upper and lower jaws, the lower jaw may be contacted with the target tissue or blood vessel first. Accordingly, by disposing the optical module 5120 in the upper jaw, the required distance between the target tissue and the optical module 5120 may be easily secured in measurement for determining optical information on the target tissue.

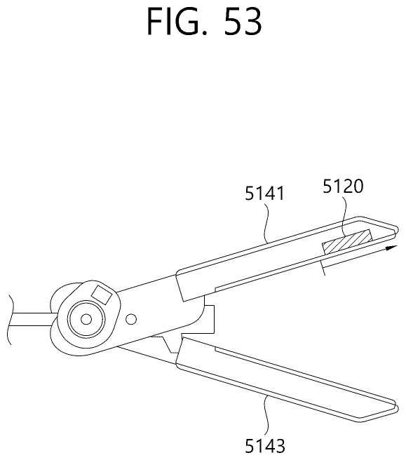

FIG. 53 is a conceptual diagram of a distal disposition of an optical unit. As illustrated in FIGS. 2 and 53, the optical unit 5120 may be disposed in the distal end of either the first jaw 5141 or the second jaw 5143. For example, the optical unit 5120 may be disposed in the distal end of the first jaw 5141. Herein, the proximal end of the end tool may refer to a gripping unit of the handpiece or a direction of the fastening unit for robot arm combination, and the distal end of the end tool may refer to the direction of the distal end of a surgical instrument, for example in the first jaw 5141 or the second jaw 5143. The optical unit 5120 may, for example, be disposed close to the distal end of the first jaw 5141, for example, disposed towards the distal end of the first jaw 5141 relative to the midpoint of the gripping unit relative to the target tissue of the first jaw 5141. During a surgical procedure, the area where the target tissue is actually contacted may differ from the tissue grippable area of the first jaw or the second jaw due to various factors such as the shape of the target tissue or the operator's habits. However, regardless of whether the target tissue is small or large, there is a very high probability that the distal end of the first jaw of the first jaw or the second jaw of tissue-contactable areas will pass the target tissue area. Accordingly, the optical unit 5120 may be disposed, for example, on the distal end of the first jaw or the second jaw to increase the probability that light from the optical unit will be transmitted to the target tissue and the probability that response light from the target tissue will be collected by the optical unit.

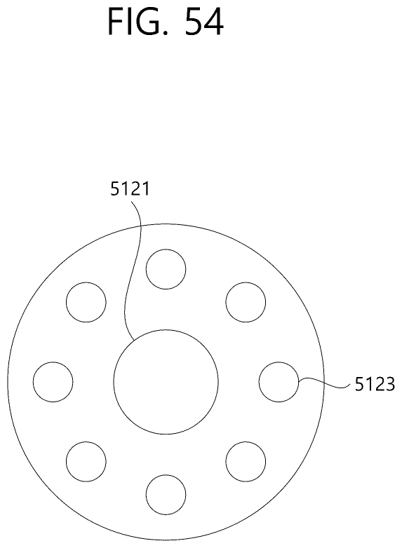

As a non-limiting but more specific example, the optical module 5120 may include, for example, one light emitting unit 5121 and a plurality of light absorption units 5123. In this regard, FIG. 54 is an exemplary diagram of the disposition of a light-emitting unit and a light-absorption unit of the optical unit. FIG. 54 exemplarily illustrates the optical module 5120 disposed, for example, on the surface facing the second jaw 5143 of the first jaw 5141. As illustrated in FIG. 54, for example, the light emitting unit 5121 may be disposed in the center of the optical module, and the plurality of light absorption units 5123 may be disposed to surround the light emitting unit 5121.

As described above, the optical unit 5120 may include the light emitting unit 5121 and the light absorption unit 5123. Herein, the light emitting unit 5121 may be configured to irradiate light of a predetermined wavelength band to the target tissue, and the light absorption unit 5123 may be disposed surrounding the light emitting unit to collect reflected light from the target tissue. Accordingly, the light emitting unit 5121 may be disposed in the center of the optical module 5120 and may be oriented to face the target tissue. The direction of reflected light from the target tissue may vary depending on the shape or orientation of the light incident surface of the target tissue. Accordingly, a plurality of light absorption units 5123 are provided to surround the light emitting unit 5121, so that reflected light may be stably collected despite the variable direction of reflected light from the target tissue.

Referring again to FIG. 51 or 52, the body unit 5100a may be provided with a power source 5160, an RF energy circuit 5110 for supplying electric energy, and a spectrometer-based optical feedback system 5170. For example, the power of the power supply unit 5160 may be transmitted to the first electrode and/or the second electrode of the end tool 5100b through the RF energy circuit 5110 under the control of the control unit 5130. In other words, the power supply unit 5160 is configured to supply electric energy to the energy transmission unit 5110. As illustrated in FIG. 52, the electric energy of the RF energy circuit 5110 may be connected to the first or second electrode located on the end tool based on a power line 5221. In other words, the energy transmission unit may be connected to the power supply unit based on the power line 5221.

As illustrated in FIG. 52, the spectrometer 5170 may be configured to generate distributed light in a predetermined wavelength band and perform measurement on the response light from the target tissue. As a non-limiting but more specific example, the spectrometer-based feedback system 5170 may be provided with a laser diode driver 5171 that generates light based on power from the power supply unit 5160, and may generate light in a predetermined wavelength range. Light in a wavelength band corresponding to the component to be irradiated may be acquired based on exemplary optical elements such as a dichroic beam splitter, collimating lens, and light cone. The light in the wavelength band corresponding to the irradiation target component generated in this way may be connected to an optical unit disposed on the end tool, for example, the light emitting unit 5121, through a first optical fiber 5223a. In other words, the optical unit may be connected to the spectrometer 5170 based on an optical fiber.

Then, light from the optical unit, for example, the light absorption unit 5123, may be transmitted to the spectrometer 5170 disposed in the body unit 5100a through a second optical fiber 5223b. As a non-limiting but more specific example, the response light from the target tissue collected by the light absorption unit 5123 may be transmitted to a photo diode analog/digital converter 5175 through the second optical fiber 5223b, and analyzed by the control unit 5130. Herein, according to an aspect, the control unit 5130 may be a microprocessor, but it is not limited thereto, and it should be understood that it may be implemented by any processor capable of computing.

As described above, between the body unit 5100a and the end tool 5100b, for example, the power line 5221 for supplying electric energy and the first optical fiber 5223a and/or the second optical fiber 5223b for optical transmission and reception, a plurality of wired connections are required to be provided. Accordingly, according to an aspect, such optical fiber and power line may be configured of a single cable 5220 having a bundle structure. Accordingly, for example, when the end tool 5100b is a handpiece directly controlled by a user, work convenience may be improved compared to having multiple wired connections. Even when the end tool is applied to a robotic surgery system, it offers design advantages, for example, the implementation of joints in robot arms and/or end tools.

Unlike a typical spectrometer feedback system, according to embodiments of the present disclosure, the connection between the optical feedback system 5170 and the optical unit 5120 may be implemented by a flexible optical fiber. Accordingly, since the optical path is not implemented by an optical path having a predetermined rigidity, it is possible to provide work convenience to a worker, and it is also possible to implement a blood vessel sealing device having a joint. In addition, in end tools that are difficult to use repeatedly, it has the advantage of being able to implement an optical path while incurring a relatively low cost.

As described above, an exemplary embodiment of the surgical device 5100 for electrocautery has been described with reference to FIG. 52, but it should be understood that this is merely an example and the technical idea of the present disclosure is not limited thereto.

Hereinafter, optical information-based supply electric energy control according to embodiments of the present disclosure will be described in more detail.

As previously described with reference to FIGS. 51 to 52, the surgical device 5100 for electrocautery according to an embodiment of the present disclosure may include the energy transmission unit 5110, the optical unit 5120, and the control unit 5130.

The energy transmission unit 5110 may be configured to transmit electric energy to the target tissue to cauterize the target tissue. For example, as described above, the energy transmission unit 5110 may include the first electrode 5111 or the second electrode 5113.

The optical unit 5120 may be configured to irradiate light in a predetermined wavelength band to the target tissue and collect response light from the target tissue. According to an aspect, light in the predetermined wavelength band may be generated using the spectrometer 5170, and analysis of the response light collected through the spectrometer 5170 or the control unit 5130 may be performed. Additionally, as described above, the optical unit 5120 may include the light emitting unit 5121 and/or the light absorption unit 5123.

The control unit 5130 may be configured to determine tissue component information on the target tissue based on the measured value of the response light and to control the electric energy supplied to the target tissue through the energy transmission unit based on the tissue component information. In other words, according to an aspect of the present disclosure, the control unit 5130 may determine component information on a specific component included in the target tissue based on optical measurement information on the target tissue. As such, the electric energy supplied to cauterize the target tissue may be controlled by reflecting information on the presence or content of a specific component in the target tissue.

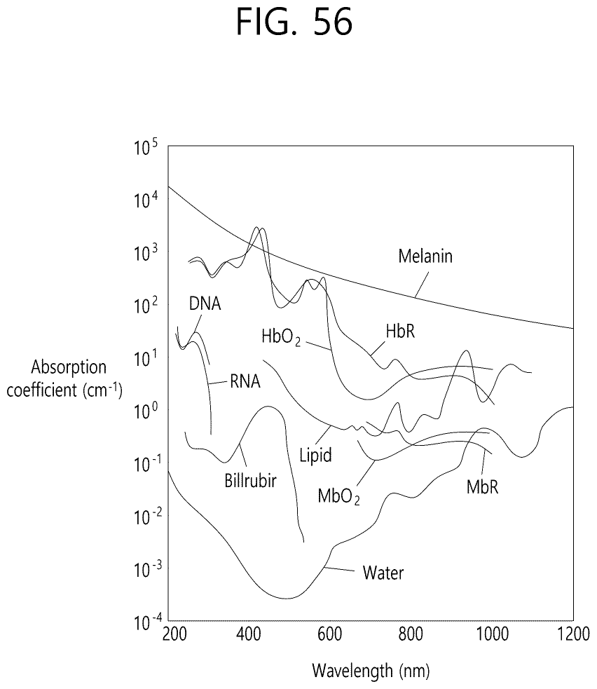

In this regard, each component of tissue within the human body may have the property of absorbing unique light (for example, a specific wavelength). FIG. 56 shows an optical absorption coefficient according to a tissue component of an exemplary target tissue.

As illustrated in FIG. 56, tissues within the human body may contain various constituents, such as moisture or lipids, and these constituents have different absorption coefficients depending on the wavelength of light. For example, a method to distinguish the difference between Hb and HbO₂ may be utilized to determine oxygen saturation. When an amount of oxygen in the body and pulse rate are measured, it is possible to irradiate the body with a specific wavelength, for example, light in the wavelength band in which Hb has a high absorption coefficient and light in the wavelength band in which HbO₂ has the highest absorption coefficient, and to determine the ratio of Hb and HbO₂ by measuring the intensity of reflected light.

According to an embodiment of the present disclosure, light in a wavelength band corresponding to a target component for determining content information among a plurality of constituents included in the target tissue is irradiated to the target tissue, and response light from the target tissue is collected. Thus, by comparing the intensity of the light irradiated to the target tissue and the response light from the target tissue, the content information of the above target component of the target tissue may be determined.

As a non-limiting but more specific example, when a laser with a wavelength in a band where changes in at least one of moisture, protein, and fat in the target tissue may be observed is used, compared to determining the supply electric energy using only the conventional impedance value, much more detailed control is possible.

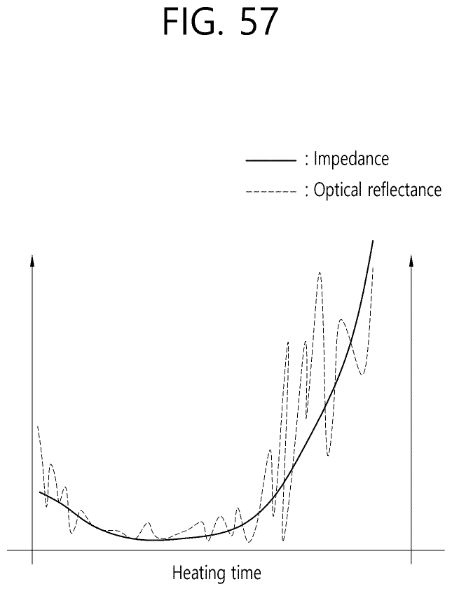

When light is irradiated to the target tissue through the light emitting unit 5121, a portion of the light within the target tissue may be absorbed by the corresponding constituent, and the remaining light may be reflected. This reflected light may be collected through the light absorption unit 5123, and the amount of change between the irradiated light and the reflected light may be calculated to determine the content information of the constituents. Using the amount of change between the irradiated light and the reflected light, changes in the content of the constituents may also be checked. For example, when the constituent is moisture, it is possible to check the change in moisture during sealing. When moisture in the tissue evaporates due to heat, the intensity of the reflected light in the wavelength band absorbed by the moisture may appear higher than when there is moisture. In this regard, FIG. 57 shows the relationship between the tissue change due to heat and the optical reflectivity and impedance accordingly. As the heating time due to the supply of electric energy increases, the impedance increases, and in relation thereto, the optical reflectance repeats its fluctuations, but ultimately tends to increase similar to the impedance.

In this regard, according to an aspect of the present disclosure, the target tissue to which electric energy is transmitted may include a first component, and this first component may correspond to a first wavelength. As a non-limiting but more specific example, the first component may have the highest optical absorption rate in the first wavelength band. Under these conditions, the control unit 5130 may be configured to determine content information on the first component of the target tissue based on the intensity of the light of the first wavelength irradiated to the target tissue and the intensity of the light of the first wavelength of the response light from the target tissue.

As a non-limiting example, the first component may be moisture. Herein, for example, moisture may correspond to a wavelength band of 1,010 nm to 1,460 nm. In other words, moisture may have the highest optical absorption rate in the wavelength band of 1,010 nm to 1,460 nm, without being limited thereto. Accordingly, in order to determine content information on moisture in the target tissue, the control unit 5130 may, for example, cause light having a wavelength of at least a portion of the wavelength band of 1,010 nm to 1,460 nm to be irradiated to the target tissue, collect the response light from the target tissue, and calculate information on the intensity difference between the light irradiated to the target tissue and the light reflected from the target tissue. Thus, it is possible to determine information on the moisture content contained in the target tissue.

As described above, the electric energy that needs to be supplied to the target tissue may vary depending on the content of a specific component contained in the target tissue. For example, when the target tissue has a high moisture content, the impedance measurement value for the target tissue may be low. In this connection, the thickness of the target tissue predicted through the impedance measurement value may be determined to be thin compared to the thickness of the actual target tissue. When the moisture content of the target tissue is high, the supply level of electric energy may be set to be relatively increased. For example, when the target tissue has a high fat content, a high level of heat may be generated by the fat even when the same electric energy is supplied to the target tissue as compared to when the fat content is low. Accordingly, according to an aspect, when the fat content of the target tissue is high, the supply level of electric energy may be set to be relatively reduced.

In this regard, according to an aspect of the present disclosure, the target tissue to which electric energy is transmitted includes a first component and a second component, wherein the first component may correspond to a first wavelength, and the second component may correspond to a second wavelength. As a non-limiting but more specific example, the first component may have the highest optical absorption rate in a first wavelength band, and the second component may have the highest optical absorption rate in a second wavelength band. Under these conditions, the control unit 5130 may be configured to increase the supply level of electric energy through the energy transmission unit 5110 in response to the increase in the content of the first component of the target tissue, and to decrease the supply level of electric energy through the energy transmission unit 5110 in response to the increase in the content of the second component of the target tissue. In other words, the control unit 5130 may be configured to increase the supply level of electric energy when the content of a specific component is high and to decrease the supply level of electric energy when the content of another specific component is high. Herein, as a non-limiting but more specific example, the first component may include moisture and the second component may include fat. Herein, for example, moisture may correspond to a wavelength band of 1,010 nm to 1,460 nm. In other words, moisture may have the highest optical absorption rate in the wavelength band of 1,010 nm to 1,460 nm, without being limited thereto. Accordingly, in order to determine content information on moisture in the target tissue, the control unit 5130 may, for example, cause light having a wavelength of at least a portion of the wavelength band of 1,010 nm to 1,460 nm to be irradiated to the target tissue, collect the response light from the target tissue, and calculate information on the intensity difference between the light irradiated to the target tissue and the light reflected from the target tissue. Thus, it is possible to determine information on the moisture content contained in the target tissue. In addition, for example, fat may correspond to a wavelength band of 900 nm to 950 nm. In other words, fat may have the highest optical absorption rate in the wavelength band of 900 nm to 950 nm, but is not limited thereto. Accordingly, in order to determine the content information on fat in the target tissue, the control unit 5130 may, for example, cause light having a wavelength of at least a portion of the wavelength band of 900 nm to 950 nm to be irradiated to the target tissue, collect the response light from the target tissue, and calculate information on the intensity difference between the light irradiated to the target tissue and the light reflected from the target tissue. Thus, it is possible to determine the content information on fat contained in the target tissue.

The wavelength band corresponding to a specific composition component may change depending on temperature changes. In other words, the wavelength band in which a specific composition component shows the highest optical absorption rate may change depending on the temperature of the object containing the composition component and/or the medium on the light transmission path. Accordingly, according to an aspect of the present disclosure, the first wavelength corresponding to the first component and/or the second wavelength corresponding to the second component will be configured to decrease with the passage of time of irradiation of electric energy to the target tissue. For example, assuming that moisture has the highest optical absorption rate at a wavelength of 1,200 nm at room temperature, when the temperature of the target tissue and/or the surrounding air, which is a light transmission medium, increases by a certain level due to electric energy, it may be changed to have the highest optical absorption rate at a wavelength of 1,000 nm, which is reduced from 1,200 nm. Since the temperature of the target tissue also increases as the irradiation time of electric energy passes, according to an aspect of the present disclosure, the first wavelength band corresponding to the first component (for example, moisture) may be reduced to a lower wavelength band corresponding to the passage of the irradiation time of the electric energy. For example, the light irradiated to the target tissue by the optical unit 5120 may be set to have a lower wavelength as the transmission time of electric energy passes. Accordingly, since light in a wavelength band in which the first component (for example, moisture) has a higher optical absorption rate is irradiated, information on the content of the first component may be determined more precisely.

According to an aspect of the present disclosure, the electric energy transmitted to the target tissue through the energy transmission unit 5110 may be controlled by using the impedance of the target tissue and tissue component information based on optical information on a specific component of the target tissue. In other words, the control unit 5130 may be configured to determine the impedance of the target tissue and control electric energy based on the impedance of the target tissue and tissue component information. Herein, as for the impedance of the target tissue, for example, as previously described in this description, at least some of current, voltage, resistance, impedance, and temperature may be measured by one or more sensors (not shown) formed on at least one of the first jaw or the second jaw. Alternatively, without a separate sensor, at least some of current, voltage, resistance, impedance, and temperature may be monitored and controlled directly by the generator (not shown) that supplies power to the electrode. In other words, the impedance of the target tissue while electric energy is supplied to the target tissue may be determined by certain constituents of the surgical device 5100 for electrocautery according to an embodiment of the present disclosure. The control unit 5130 may control the electric energy supplied to the target tissue by using both the information on the impedance of the target tissue determined as such and the content information on a specific component based on optical information described above in this description.

As a non-limiting example, the control unit 5130 may be configured to adjust the electric energy level corresponding to the impedance of the target tissue based on tissue component information. More specifically, for example, assuming a case where the target tissue includes a first component and a second component, the control unit 5130 may be configured to determine the reference electric energy based on the impedance of the target tissue, and to determine the electric energy supplied to the target tissue by the energy transmission unit 5110 by increasing the reference electric energy based on information on the first component content or decreasing the reference electric energy based on information on the second component content.

As in a conventional surgical device for electrocautery, the control unit 5130 may first determine the supply electric energy to the target tissue based on the impedance measured for the target tissue, without considering tissue component information of the target tissue. As such, the electric energy as a supply target determined using only impedance may be the reference electric energy. Thereafter, the control unit 5130 may determine information on the content of a specific composition component based on optical information as described above in this description.

When the content of a specific first component (for example, moisture) is high, the impedance may be measured to be low compared to the thickness of the target tissue. Accordingly, the reference electric energy determined without considering the content information of the first component may be at a lower level than the actual required electric energy. Accordingly, the control unit 5130 may be configured to acquire information on the content of the first component of the target tissue and determines the electric energy supplied to the target tissue by increasing the calculated reference electric energy when the content of the first component is high. Accordingly, by ensuring that an appropriate level of electric energy is supplied to the target tissue, it is possible to quickly perform a cauterization procedure on the target tissue while preventing tissue damage.

When the content of a specific other second component (for example, fat) is high, heat higher than the target temperature may be applied to the target tissue even when the same electric energy is supplied due to the high heat generation rate of the fat. Accordingly, the control unit 5130 may be configured to acquire information on the content of the second component of the target tissue and determines the electric energy supplied to the target tissue by decreasing the calculated reference electric energy when the content of the second component is high. Accordingly, by ensuring that an appropriate level of electric energy is supplied to the target tissue, damage to the tissue may be prevented.

As described above, the surgical device 5100 for electrocautery according to an embodiment of the present disclosure determines tissue component information of the target tissue based on optical information and further reflects the same to control the electric energy transmitted to the target tissue. Thereby, it is possible to finely distinguish between thin target tissue and high moisture content. Accordingly, for example, in response to the impedance being lowered and the water content in the target tissue showing a low value, it may be determined that the tissue currently being gripped by the end tool is in a very thin state. In such a situation, unnecessary tissue damage or increased sealing time may be prevented by terminating the RF energy without further application to the target tissue.

In this regard, according to an aspect of the present disclosure, when the target tissue includes the first component, the control unit 5130 may be configured to discontinue the supply of electric energy to the energy transmission unit in response to a determination that the impedance of the target tissue is less than or equal to a first predetermined threshold and the first component content is less than or equal to a second predetermined threshold. In other words, when not only the impedance value measured for the target tissue is low, but also the content of the first component (for example, moisture) is also low, it may be determined more reliably that the target tissue is thin.

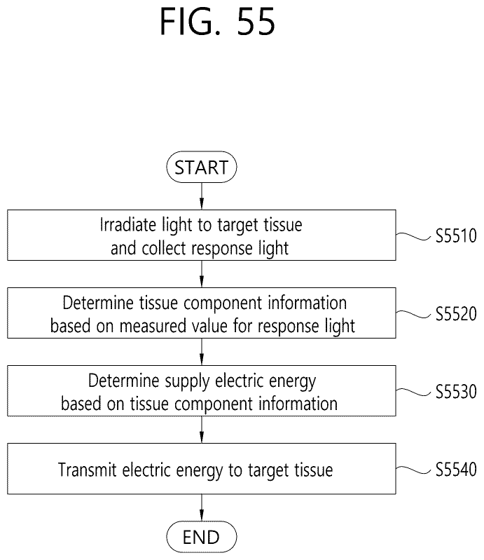

FIG. 55 is a schematic flow diagram of a method for determining supply electric energy of the surgical device for electrocautery according to an embodiment of the present disclosure. The method for determining the supply electric energy of the surgical device for electrocautery may be performed, for example, by a computing device. Herein, the computing device may refer to any arithmetic device including a processor and memory, or may refer to a processor itself capable of computing. Alternatively, the computing device herein may be a microprocessor or the control unit 5130 described herein as a configuration of the surgical device 5100 for electrocautery. Hereinafter, for convenience of explanation, it is exemplified that the method for determining the supply electric energy of the surgical device for electrocautery according to an aspect of the present disclosure is performed by a computing device, without being limited thereto.

As illustrated in FIG. 55, the computing device may first irradiate light in a predetermined wavelength band to a target tissue using an optical unit and collect response light from the target tissue (stage 5510).

Thereafter, the computing device may determine tissue component information on the target tissue based on the measured value for the response light (stage 5520).

Once the tissue component information is determined, the computing device may be set to determine the supply electric energy to the target tissue through the energy transmission unit based on the tissue component information (stage 5530), and to transmit the determined electric energy to the target tissue using the energy transmission unit (stage 5540).

A more specific procedure of the method for determining the supply electric energy according to an embodiment of the present disclosure may employ at least a portion of the motion procedure described in this description with respect to the surgical device 5100 for electrocautery according to an embodiment of the present disclosure.

The aforementioned method according to an embodiment of the present application may be implemented as computer-readable code stored on a computer-readable recording medium. The computer-readable recording medium includes all types of recording media storing data that may be deciphered by a computer system. Examples of the computer-readable recording medium include a read only memory (ROM), a random access memory (RAM), magnetic tapes, magnetic disks, a flash memory, and optical data storage devices. The computer-readable recording medium may also be distributed over a computer system connected through a computer communication network, and stored and executed as a code that may be read in a distributed manner.

The aforementioned method may be included and provided in a computer program product. The computer program product may be traded between a seller and a buyer as commodities. The computer program product may be distributed in a form of the machine-readable storage media (for example, compact disc read only memory (CD-ROM)) or distributed (for example, downloaded or uploaded) online through an application store (for example, PlayStore™) or directly between two user devices. In the case of the online distribution, at least a portion of the computer program product may be at least temporarily stored or provisionally generated on the machine-readable storage media, such as a manufacturer's server, an application store's server, or a memory in a relay server.

Hereinbefore, the embodiments of the present disclosure has been described with reference to the accompanying drawing, but the scope of protection of the present disclosure should not be construed as being limited to the drawings or embodiments. It will be understood by those skilled in the technical field that the present disclosure allows various modifications and variations without departing from the scope and spirit of the present disclosure as described in the claims below.

Specifically, the described features may be implemented within digital electronic circuitry, or computer hardware, firmware, or combinations thereof. The features may be implemented in a computer program product embodied in a storage device in a machine-readable storage device, for example, for execution by a programmable processor. In addition, the features may be performed by a programmable processor executing a program of instructions for performing functions of the described embodiments, by operating on input data and generating an output. The described features may be implemented in one or more computer programs that are executable on a programmable system including at least one programmable processor coupled to receive data and instructions from, and to transmit data and instructions to, a data storage system, at least one input device, and at least one output device. A computer program is a set of instructions that may be used, directly or indirectly, in a computer to perform a certain motion for a certain result. A computer program may be written in any form of programming language, including compiled or interpreted languages, and may be deployed in any form, including as a stand-alone program or as a module, component, subroutine, or other unit suitable for use in a computing environment.

Suitable processors for the execution of a program of instructions include, by way of example, both general and special purpose microprocessors, and a sole processor or one of multiple processors of any kind of computer. In addition, storage devices suitable for implementing computer program instructions and data implementing the described features include all forms of non-volatile memory, including, by way of example, semiconductor memory devices, such as EPROM, EEPROM, and flash memory devices; magnetic devices, such as internal hard disks and removable disks; magneto-optical disks; and CD-ROM and DVD-ROM disks. The processor and the memory may be supplemented by, or incorporated in, ASICs (application-specific integrated circuits).

While the present disclosure has been described on the basis of a series of functional blocks, it is not limited by the embodiments described above and the accompanying drawings and it will be apparent to those skilled in the art that various substitutions, modifications and variations may be made without departing from the scope of the present disclosure.

The combination of the above-described embodiments is not limited to the above-described embodiments, and various forms of combination in addition to the above-described embodiments may be provided according to implementation and/or necessity.

In the above-described embodiments, the methods are described on the basis of a flowchart as a series of operations or blocks, but the present disclosure is not limited to the order of the operations, and some operations may occur in different orders or at the same time unlike those described above. It will also be understood by those skilled in the art that the operations shown in the flowchart are not exclusive, and other operations may be included, or one or more operations in the flowchart may be omitted without affecting the scope of the present disclosure.

The above-described embodiments include examples of various aspects. While it is not possible to describe every possible combination for expressing various aspects, one of ordinary skill in the art will recognize that other combinations are possible. Accordingly, it is intended that the present disclosure include all alternatives, modifications and variations that fall within the scope of the following claims.

What is claimed is:

1. A surgical device for electrocautery, comprising:
at least one energy transmission unit configured to transmit electric energy to a target tissue to cauterize the target tissue;
an optical unit configured to irradiate light of a predetermined wavelength band to the target tissue and collect response light from the target tissue; and
a control unit configured to determine tissue component information on the target tissue based on a measurement value for the response light and to control electric energy supplied to the target tissue through the energy transmission unit based on the tissue component information.

2. The device of claim 1, wherein:
the target tissue comprises a first component;
the first component corresponds to a first wavelength; and
the control unit is configured to determine content information on the first component of the target tissue based on an intensity of light of the first wavelength irradiated to the target tissue and an intensity of light of the first wavelength of the response light from the target tissue.

3. The device of claim 1, wherein:
the target tissue comprises a first component and a second component; and
the control unit is configured to increase a supply level of the electric energy in response to an increase in a content of a first component of the target tissue, and to decrease the supply level of the electric energy in response to an increase in a content of a second component of the target tissue.

4. The device of claim 3, wherein:
the first component comprises moisture; and
the second component comprises fat.

5. The device of claim 4, wherein:
the moisture corresponds to a wavelength band of 1,010 nm to 1,460 nm; and
the fat corresponds to a wavelength band of 900 nm to 950 nm.

6. The device of claim 2, wherein the first wavelength is configured to decrease with the passage of electric energy irradiation time to the target tissue.

7. The device of claim 1, wherein the control unit is configured to determine an impedance of the target tissue and control the electric energy based on the impedance of the target tissue and the tissue component information.

8. The device of claim 7, wherein the control unit is configured to adjust an electric energy level corresponding to the impedance of the target tissue based on the tissue component information.

9. The device of claim 7, wherein:
the target tissue comprises a first component and a second component; and
the control unit determines a reference electric energy based on the impedance of the target tissue, and determines the electric energy supplied to the energy transmission unit by increasing the reference electric energy based on information on a content of the first component or decreasing the reference electric energy based on information on a content of the second component.

10. The device of claim 7, wherein:
the target tissue comprises a first component; and
the control unit is configured to stop supplying electric energy to the energy transmission unit in response to a determination that the impedance of the target tissue is less than or equal to a predetermined first threshold value and a content of the first component is less than or equal to a predetermined second threshold value.

11. The device of claim 1, further comprising: a first jaw for gripping the target tissue; and a second jaw,
wherein the energy transmission unit comprises a first electrode provided in the first jaw and a second electrode provided in the second jaw.

12. The device of claim 1, further comprising a first jaw and a second jaw for gripping the target tissue,
wherein the optical unit is disposed in either the first jaw or the second jaw and configured to irradiate light of the predetermined wavelength band to the target tissue and collect reflected light from the target tissue.

13. The device of claim 12, further comprising a blade that cut the target tissue while moving between a proximal end and a distal end of either the first jaw or the second jaw.

14. The device of claim 13, wherein the optical unit is disposed in the first jaw, and the blade is disposed in the second jaw.

15. The device of claim 12, wherein the optical unit is disposed in a distal end of either the first jaw or the second jaw.

16. The device of claim 12, wherein the optical unit comprises:
a light emitting unit that irradiates light of the predetermined wavelength band to the target tissue; and
at least one absorption unit that is disposed around the light emitting unit and configured to collect reflected light from the target tissue.

17. The device of claim 1, further comprising a spectrometer that generates dispersed light of the predetermined wavelength band and performs a measurement on the response light,
wherein the optical unit is connected to the spectrometer based on an optical fiber.

18. The device of claim 17, further comprising a power supply unit that supplies electric energy to the energy transmission unit,
wherein the energy transmission unit is connected to the power supply unit based on a power line.

19. The device of claim 17, wherein the optical fiber and the power line are configured as a single cable having a bundle structure.

20. A method for determining supply electric energy of a surgical device for electrocautery, performed by a computing device, the method comprising:
irradiating light of a predetermined wavelength band to a target tissue using an optical unit and collecting response light from the target tissue;
determining tissue component information on the target tissue based on a measurement value for the response light;
determining electric energy supplied to the target tissue through an energy transmission unit based on the tissue component information; and
transmitting the determined electric energy to the target tissue using the energy transmission unit.
